(19) United States
(12) Patent Application Publication
(10) Pub. No.: US 2024/0415103 A1
(43) Pub. Date: Dec. 19, 2024

(54) VECTORS, GENETICALLY MODIFIED CELLS, AND GENETICALLY MODIFIED NON-HUMAN ANIMALS COMPRISING THE SAME

(71) Applicant: Regeneron Pharmaceuticals, Inc., Tarrytown, NY (US)

(72) Inventors: Davor Frleta, Tarrytown, NY (US); John McWhirter, Tarrytown, NY (US)

(21) Appl. No.: 18/744,415

(22) Filed: Jun. 14, 2024

Related U.S. Application Data
(60) Provisional application No. 63/521,475, filed on Jun. 16, 2023.

(51) Int. Cl. A01K 67/0275; A61K 49/00; C12N 5/0735; C12N 9/12; C12N 15/85

(52) U.S. Cl. CPC A01K 67/0275; A61K 49/0008; C12N 5/0606; C12N 9/12; C12N 9/1241; C12N 15/8509; C12Y 207/10001; A01K 2217/052; A01K 2217/054; A01K 2227/105; A01K 2267/03; C12N 2015/8527; C12N 2800/107

(57) ABSTRACT

Provided herein are genetically modified cells and genetically modified non-human animals (e.g., rodents such as rats and mice) comprising: (i) a homozygous null mutation in Rag2 gene; (ii) a homozygous null mutation in IL2rg gene; (iii) a homozygous null mutation in the non-human animal FMS-like tyrosine kinase 3 (Flt3) gene, and (iv) a Flt3 ligand (Flt31) gene that comprises a non-human animal portion and a human portion operably linked to a Flt31 promoter, and optionally expressing one or more human or humanized polypeptides. Methods and compositions of making and using such genetically modified cells and non-human animals are also provided.

### RELATED APPLICATIONS

This application claims priority to U.S. Provisional Application No. 63/521,475, filed Jun. 16, 2023, which is hereby incorporated herein by reference in its entirety.

### SEQUENCE LISTING

The instant application contains a Sequence Listing which has been submitted electronically in XML format and is hereby incorporated by reference in its entirety. Said XML copy, created on Jun. 14, 2024, is named RPD-00201.xml and is 43,711 bytes in size.

### BACKGROUND

Genetically modified cells, genetically modified mice comprising the same, as well as modified and engrafted mice, and their use in modeling human diseases, e.g., for the purpose of drug testing, are known in the art. Use of genetically modified mice to model a human immune system (HIS) has been reported (Manz (2007) Immunity, 26:537-541). For example, HIS mice generated by transplanting a severely immunodeficient mouse strain (such as Rag2 KO Il2rg KO mice) with human hematopoietic stem and progenitor cells have been reported. Although multi-lineage hematopoietic development has been observed in these HIS mice, there is a need for genetically modified mice that can support human dendritic cell development, and for mice suitable for engraftment that can model or approximate certain aspects of a human dendritic cells.

### SUMMARY

The present disclosure is based, in part, on the discovery that knocking out Flt3 gene in immune-compromised mice (e.g., mice having a Rag1 and/or Rag2 gene knockout and an IL2rg gene knockout) with humanization of Flt31 led to increased human DCs (e.g., myeloid DCs, plasmacytoid DCs, BDCA-3+ DCs) in the spleen, blood and bone marrow after the human hematopoietic cell engraftment.

Accordingly, in one aspect, the present disclosure relates to a genetically modified rodent, comprising: (i) a homozygous null mutation in Rag2 gene; (ii) a homozygous null mutation in IL2rg gene; (iii) a homozygous null mutation in the rodent FMS-like tyrosine kinase 3 (Flt3) gene; and (iv) a Flt3 ligand (Flt31) gene that comprises a rodent portion and a human portion operably linked to a Flt31 promoter.

In some embodiments, the genetically modified rodent comprises a homozygous null mutation in Rag1 gene. In some embodiments, the null mutation in the rodent Flt3 gene comprises an insertion, a deletion, and/or a substitution in the endogenous Flt3 gene. In some embodiments, the null mutation in the rodent Flt3 gene is a deletion of the full Flt3 endogenous coding sequence. In some embodiments, the genetically modified rodent is a mouse, and the mouse comprises a homozygous deletion of nucleic acid sequence between coordinates chr5: 147331171-147400265 (GRCm38 assembly).

In some embodiments, the rodent portion of the Flt31 gene comprises exon 1, a non-coding portion of exon 2, and exons downstream of exon 6 of a rodent Flt31 gene. In some embodiments, wherein the exon 1, the non-coding portion of exon 2, and the exons downstream of exon 6 of the rodent Flt31 gene are at least 90%, at least 95%, or 100% identical to the corresponding exon 1, non-coding portion of exon 2, and exons downstream of exon 6 of a rodent Flt31 gene shown in Table 1A. In some embodiments, the human portion of the Flt31 gene comprises a signal peptide-coding portion of exon 2, and exons 3-6 of a human FLT3L gene. In some embodiments, the signal peptide-coding portion of exon 2, and exons 3-6 of the human FLT3L gene are at least 50%, at least 60%, at least 70%, at least 80%, at least 90%, at least 95%, or 100% identical to the corresponding signal peptide-coding portion of exon 2, and exons 3-6 of a human FLT3L gene shown in Table 1B. In some embodiments, the Flt31 gene comprises rodent exon 1, rodent non-coding portion of exon 2, human signal peptide-coding portion of exon 2, human exons 3-6, and rodent exons 7-9.

In some embodiments, the Flt31 gene encodes a chimeric membrane bound Flt31 comprising a signal peptide and cytokine-like core domain of a human FLT3L polypeptide, and a C-terminal portion of a rodent Flt31 polypeptide. In some embodiments, the C-terminal portion of the rodent Flt31 polypeptide comprises a rodent stalk region, a rodent transmembrane domain, and a rodent cytoplasmic tail. In some embodiments, the C-terminal portion of the rodent Flt31 polypeptide has an amino acid sequence that is at least 70%, at least 80%, at least 90%, at least 95%, or 100% identical to a C-terminal portion of a rodent Flt31 polypeptide shown in Table 2. In some embodiments, the rodent expresses both soluble and membrane-bound forms of the Flt31 polypeptide. In some embodiments, the soluble and membrane-bound forms of the Flt31 polypeptide comprise a signal peptide and a cytokine-like core domain of a human FLT3L polypeptide. In some embodiments, the signal peptide of the human FLT3L polypeptide comprises amino acids corresponding to residues 1-26 of a human FLT3L polypeptide. In some embodiments, the signal peptide of the human FLT3L polypeptide comprises an amino acid sequence that is at least 70%, at least 80%, at least 90%, at least 95%, or 100% identical to a corresponding amino acid sequence of a signal peptide of a human FLT3L polypeptide that appears in Table 2. In some embodiments, the cytokine-like core domain of the human FLT3L polypeptide comprises amino acids corresponding to residues 27-159 of a human FLT3L polypeptide. In some embodiments, the cytokine-like core domain of the human FLT3L polypeptide comprises an amino acid sequence that is at least 70%, at least 80%, at least 90%, at least 95%, or 100% identical to a corresponding amino acid sequence of cytokine-like core domains of a human FLT3L polypeptide that appears in Table 2.

In some embodiments, the genetically modified rodent is heterozygous for the Flt3 ligand (Flt31) gene that comprises a rodent portion and a human portion. In some embodiments, the genetically modified rodent is homozygous for the Flt3 ligand (Flt31) gene that comprises a rodent portion and a human portion.

In some embodiments, the rodent portion of the Flt31 gene is an endogenous rodent portion of the Flt31 gene; the rodent Flt31 gene is an endogenous rodent gene; and/or the rodent Flt31 polypeptide is an endogenous rodent Flt31 polypeptide.

In some embodiments, the rodent and human portions are operably linked to a Flt31 promoter. In some embodiments, the Flt31 promoter is a rodent promoter. In some embodiments, the Flt31 promoter is an endogenous rodent promoter. In some embodiments, the Flt31 promoter is at the endogenous rodent gene locus.

In some embodiments, the genetically modified rodent expresses a human or humanized SIRPA polypeptide encoded by a nucleic acid operably linked to a Sirpa promoter. In some embodiments, the genetically modified rodent further comprises a Sirpa gene that encodes a Sirpa polypeptide comprising an extracellular portion of a human SIRPA polypeptide and an intracellular portion of a rodent Sirpa polypeptide, wherein the Sirpa gene is operably linked to a Sirpa promoter. In some embodiments, the Sirpa gene comprises exons 1, 5, 6, 7 and 8 of a rodent Sirpa gene and exons 2-4 of a human SIRPA gene. In some embodiments, the genetically modified rodent further expresses a Sirpa polypeptide comprising an extracellular portion of a human SIRPA polypeptide and an intracellular portion of a rodent Sirpa polypeptide. In some embodiments, the rodent Sirpa polypeptide is an endogenous rodent Sirpa polypeptide, and/or the rodent Sirpa gene is an endogenous rodent gene. In some embodiments, the genetically modified rodent further expresses a human SIRPA polypeptide encoded by a nucleic acid operably linked to a Sirpa promoter.

In some embodiments, the genetically modified rodent further expresses a human GM-CSF protein encoded by a nucleic acid operably linked to a GM-CSF promoter and/or a human IL3 protein encoded by a nucleic acid operably linked to a IL3 promoter.

In some embodiments, the Sirpa promoter, the GM-CSF promoter, and/or the IL3 promoter is a rodent promoter. In some embodiments, the Sirpa promoter, the GM-CSF promoter, and/or the IL3 promoter is an endogenous rodent promoter. In some embodiments, the Sirpa promoter, the GM-CSF promoter, and/or the IL3 promoter is at the corresponding endogenous rodent gene locus.

In some embodiments, the genetically modified rodent described herein comprises a null mutation in at least one corresponding rodent gene at the corresponding rodent gene locus.

In some embodiments, the genetically modified rodent is heterozygous for at least one allele comprising the nucleic acid sequence that encodes the human or humanized protein. In some embodiments, the genetically modified rodent is homozygous for at least one allele comprising the nucleic acid sequence that encodes the human or humanized protein.

In some embodiments, the genetically modified rodent further comprises an engraftment of human hematopoietic cells. In some embodiments, human hematopoietic cells comprise one or more cells selected from the group consisting of a human CD34-positive cell, a human hematopoietic stem cell, a human hematopoietic progenitor cell, a human dendritic cell precursor cell, and a human dendritic cell. In some embodiments, the genetically modified rodent comprises human dendritic cells.

In some embodiments, an autoimmune disease is induced or established in the genetically modified rodent. In some embodiments, the autoimmune disease is systemic lupus erythematosus, systemic sclerosis, sjogren's syndrome, polymyositis, collagen-induced arthritis, or dermatomyositis.

In some embodiments, the rodent is a mouse or a rat. In some embodiments, the rodent is a mouse.

In certain aspects, provided herein is a method of identifying an agent that modulates a function of a human dendritic cell, comprising: a. administering the candidate agent to a genetically modified rodent described herein; and b. determining whether the candidate agent modulates the function of the human dendritic cell in the rodent. In some embodiments, the human dendritic cell is a myeloid dendritic cell (mDC) or a plasmacytoid dendritic cell (pDC). In some embodiments, the function of the human dendritic cell is selected from a group consisting of phagocytosis, cytokine production, cross-presentation of exogenous antigens, and activation of cytotoxic CD8+ T cell lymphocytes (CTLs).

In certain aspects, provided herein is a method for assessing therapeutic efficacy of a drug to stimulate a T cell response to a target cell, the method comprising: a. administering a drug candidate to a genetically modified rodent described herein, wherein the genetically modified rodent comprises the target cell; and b. measuring the T cell response to the target cell in the rodent to assess the therapeutic efficacy of the drug candidate. In some embodiments, the target cell is selected from the group consisting of a tumor cell, a virally-infected cell, a bacterially-infected cell, a bacterial cell, a fungal cell, and a parasitic cell.

In certain aspects, provided herein is a method for assessing therapeutic efficacy of a drug in treating an autoimmune disease, the method comprising: a. administering the candidate agent to a genetically modified rodent described herein; and b. determining whether the agent treats the autoimmune disease in the rodent.

In another aspect, the present disclosure relates to a genetically modified rodent cell comprising: (i) a homozygous null mutation in Rag2 gene; (ii) a homozygous null mutation in IL2rg gene; (iii) a homozygous null mutation in the rodent FMS-like tyrosine kinase 3 (Flt3) gene; and (iv) a Flt3 ligand (Flt31) gene that comprises a rodent portion and a human portion operably linked to a Flt31 promoter.

In some embodiments, the genetically modified rodent cell comprises a homozygous null mutation in Rag1 gene. In some embodiments, the null mutation in the rodent Flt3 gene comprises an insertion, a deletion, and/or a substitution in the endogenous Flt3 gene. In some embodiments, the null mutation in the rodent Flt3 gene is a deletion of the full Flt3 endogenous coding sequence. In some embodiments, the genetically modified rodent cell is a mouse cell, and the mouse cell comprises a homozygous deletion of nucleic acid sequence between coordinates chr5: 147331171-147400265 (GRCm38 assembly).

In some embodiments, the rodent portion of the Flt31 gene comprises exon 1, a non-coding portion of exon 2, and exons downstream of exon 6 of a rodent Flt31 gene. In some embodiments, wherein the exon 1, the non-coding portion of exon 2, and the exons downstream of exon 6 of the rodent Flt31 gene are at least 90%, at least 95%, or 100% identical to the corresponding exon 1, non-coding portion of exon 2, and exons downstream of exon 6 of a rodent Flt31 gene shown in Table 1A. In some embodiments, the human portion of the Flt31 gene comprises a signal peptide-coding portion of exon 2, and exons 3-6 of a human FLT3L gene. In some embodiments, the signal peptide-coding portion of exon 2, and exons 3-6 of the human FLT3L gene are at least 50%, at least 60%, at least 70%, at least 80%, at least 90%, at least 95%, or 100% identical to the corresponding signal peptide-coding portion of exon 2, and exons 3-6 of a human FLT3L gene shown in Table 1B. In some embodiments, the Flt31 gene comprises rodent exon 1, rodent non-coding portion of exon 2, human signal peptide-coding portion of exon 2, human exons 3-6, and rodent exons 7-9.

In some embodiments, the Flt31 gene encodes a chimeric membrane bound Flt31 comprising a signal peptide and cytokine-like core domain of a human FLT3L polypeptide, and a C-terminal portion of a rodent Flt31 polypeptide. In some embodiments, the C-terminal portion of the rodent Flt31 polypeptide comprises a rodent stalk region, a rodent transmembrane domain, and a rodent cytoplasmic tail. In some embodiments, the C-terminal portion of the rodent Flt31 polypeptide has an amino acid sequence that is at least 70%, at least 80%, at least 90%, at least 95%, or 100% identical to a C-terminal portion of a rodent Flt31 polypeptide shown in Table 2. In some embodiments, the rodent expresses both soluble and membrane-bound forms of the Flt31 polypeptide. In some embodiments, the soluble and membrane-bound forms of the Flt31 polypeptide comprise a signal peptide and a cytokine-like core domain of a human FLT3L polypeptide. In some embodiments, the signal peptide of the human FLT3L polypeptide comprises amino acids corresponding to residues 1-26 of a human FLT3L polypeptide. In some embodiments, the signal peptide of the human FLT3L polypeptide comprises an amino acid sequence that is at least 70%, at least 80%, at least 90%, at least 95%, or 100% identical to a corresponding amino acid sequence of a signal peptide of a human FLT3L polypeptide that appears in Table 2. In some embodiments, the cytokine-like core domain of the human FLT3L polypeptide comprises amino acids corresponding to residues 27-159 of a human FLT3L polypeptide. In some embodiments, the cytokine-like core domain of the human FLT3L polypeptide comprises an amino acid sequence that is at least 70%, at least 80%, at least 90%, at least 95%, or 100% identical to a corresponding amino acid sequence of cytokine-like core domains of a human FLT3L polypeptide that appears in Table 2.

In some embodiments, the genetically modified rodent cell is heterozygous for the Flt3 ligand (Flt31) gene that comprises a rodent portion and a human portion. In some embodiments, the genetically modified rodent cell is homozygous for the Flt3 ligand (Flt31) gene that comprises a rodent portion and a human portion.

In some embodiments, the rodent portion of the Flt31 gene is an endogenous rodent portion of the Flt31 gene; the rodent Flt31 gene is an endogenous rodent gene; and/or the rodent Flt31 polypeptide is an endogenous rodent Flt31 polypeptide.

In some embodiments, the rodent and human portions are operably linked to a Flt31 promoter. In some embodiments, the Flt31 promoter is a rodent promoter. In some embodiments, the Flt31 promoter is an endogenous rodent promoter. In some embodiments, the Flt31 promoter is at the endogenous rodent gene locus.

In some embodiments, the genetically modified rodent cell further comprises a nucleic acid that encodes a human or humanized SIRPA polypeptide, and wherein the nucleic acid is operably linked to a Sirpa promoter. In some embodiments, the genetically modified rodent cell expresses a human or humanized SIRPA polypeptide. In some embodiments, the genetically modified rodent cell further comprises a Sirpa gene that encodes a Sirpa polypeptide comprising an extracellular portion of a human SIRPA polypeptide and an intracellular portion of a rodent Sirpa polypeptide, wherein the Sirpa gene is operably linked to a Sirpa promoter. In some embodiments, the Sirpa gene comprises exons 1, 5, 6, 7 and 8 of a rodent Sirpa gene and exons 2-4 of a human SIRPA gene. In some embodiments, the genetically modified rodent cell expresses a Sirpa polypeptide comprising an extracellular portion of a human SIRPA polypeptide and an intracellular portion of a rodent Sirpa polypeptide. In some embodiments, the rodent Sirpa polypeptide is an endogenous rodent Sirpa polypeptide, and/or the rodent Sirpa gene is an endogenous rodent gene. In some embodiments, the genetically modified rodent cell expresses a human SIRPA polypeptide.

In some embodiments, the genetically modified rodent cell further comprises: (1) a nucleic acid that encodes a human GM-CSF protein and is operably linked to a GM-CSF promoter; and/or (2) a nucleic acid that encodes a human IL3 protein and is operably linked to a IL3 promoter. In some embodiments, the genetically modified rodent cell expresses a human GM-CSF protein and/or a human IL3 protein.

In some embodiments, the Sirpa promoter, the GM-CSF promoter, and/or the IL3 promoter is a rodent promoter. In some embodiments, the Sirpa promoter, the GM-CSF promoter, and/or the IL3 promoter is an endogenous rodent promoter. In some embodiments, the Sirpa promoter, the GM-CSF promoter, and/or the IL3 promoter is at the corresponding endogenous rodent gene locus.

In some embodiments, the genetically modified rodent cell described herein comprises a null mutation in at least one corresponding rodent gene at the corresponding rodent gene locus.

In some embodiments, the genetically modified rodent cell is heterozygous for at least one allele comprising the nucleic acid sequence that encodes the human or humanized protein. In some embodiments, the genetically modified rodent cell is homozygous for at least one allele comprising the nucleic acid sequence that encodes the human or humanized protein.

In some embodiments, the genetically modified rodent cell is a rodent embryonic stem (ES) cell.

In certain aspects, provided herein is a method of making a rodent embryonic stem cell, comprising genetically engineering the rodent embryonic stem cell so that the rodent embryonic stem cell has a genome that comprises: (i) a homozygous null mutation in Rag2 gene; (ii) a homozygous null mutation in IL2rg gene; (iii) a homozygous null mutation in the rodent FMS-like tyrosine kinase 3 (Flt3) gene; and (iv) a Flt3 ligand (Flt31) gene that comprises a rodent portion and a human portion operably linked to a Flt31 promoter.

In certain aspects, provided herein is a rodent embryo comprising the rodent embryonic stem cell described herein, or the rodent embryonic stem cell made according to the method described herein.

In certain aspects, provided herein is a method of making a rodent comprising in its genome: (i) a homozygous null mutation in Rag2 gene; (ii) a homozygous null mutation in IL2rg gene; (iii) a homozygous null mutation in the rodent FMS-like tyrosine kinase 3 (Flt3) gene; and (iv) a Flt3 ligand (Flt31) gene that comprises a rodent portion and a human portion operably linked to a Flt31 promoter, the method comprising steps of: (a) obtaining a rodent embryonic stem cell described herein, or the rodent embryonic stem cell made according to the method described herein; and (b) creating a rodent using the rodent embryonic cell of (a).

In certain aspects, provided herein is a method of making a rodent comprising in its genome: (i) a homozygous null mutation in Rag2 gene; (ii) a homozygous null mutation in IL2rg gene; (iii) a homozygous null mutation in the rodent FMS-like tyrosine kinase 3 (Flt3) gene; and (iv) a Flt3 ligand (Flt31) gene that comprises a rodent portion and a human portion operably linked to a Flt31 promoter, the method comprising modifying the genome of the rodent so that it comprises: (i) a homozygous null mutation in Rag2 gene; (ii) a homozygous null mutation in IL2rg gene; (iii) a homozygous null mutation in the rodent FMS-like tyrosine kinase 3 (Flt3) gene; and (iv) a Flt3 ligand (Flt31) gene that comprises a rodent portion and a human portion operably linked to a Flt31 promoter.

### BRIEF DESCRIPTION OF THE DRAWINGS

FIGS. 1A-1B show generation of final targeting vector (modified BAC #3) for mouse Flt31 (referred to as “mFlt3l” or “Flt3l” in FIGS. 1A and 1B) humanization. Unless labeling in the diagrams suggests otherwise (e.g., as for selection cassettes, loxP sites, etc.), filled shapes and single lines represent mouse sequences, and empty shapes and double lines represent human sequences. “SPEC” indicates spectinomycin resistance gene; “EM7-Hyg” indicates a hygromycin resistant gene with EM7 promoter; “loxP” indicates a lox P site; SDC NEO is a neomycin self-deleting cassette. Human FLT3L is referred to as “FLT3LG” in FIG. 1B.

FIGS. 1C-1D show schematic summaries, not to scale, of exemplary modified mouse Flt31 (referred to as “mFLT31” or “Flt3l” in FIGS. 1C and 1D) loci according to certain exemplary embodiments provided herein. FIG. 1C shows the modified mouse Flt31 loci before the removal of the loxp-Neo-loxp self-deleting cassette.

FIG. 1D shows the modified mouse Flt31 loci after the removal of the loxp-Neo-loxp self-deleting cassette. Unless labeling in the diagrams suggests otherwise (e.g., as for selection cassettes, loxP sites, etc.), filled shapes and single lines represent mouse sequences, and empty shapes and double lines represent human sequences. “loxP” indicates a lox P site; SDC NEO is a neomycin self-deleting cassette. Human FLT3L is referred to as “FLT3LG” or “hFLT3LG” in FIGS. 1C and 1D.

FIG. 1E shows sequence alignments among mouse membrane bound Flt31 (mFlt31_MB) (SEQ ID NO: 31), chimeric membrane bound FLT3L (Chimeric_FLT3LG_MB) (SEQ ID NO: 11), human membrane bound FLT3L (hFLT3LG_MB) (SEQ ID NO: 32). Specific protein domains are boxed and labeled; triangles represent locations of exon boundaries of corresponding nucleic acid sequences of mouse or human FLT3LG gene (top or filled triangles are mouse exon boundaries and bottom or empty triangles are human exon boundaries) that encode depicted mouse or human proteins.

FIG. 2 shows schematic summaries, not to scale, of generation of exemplary modified mouse Flt3 loci (i.e., Flt3 deletion) according to certain exemplary embodiments provided herein. “loxP” indicates a lox P site; SDC HYG is a hygromycin self-deleting cassette.

FIG. 3 shows high levels of humanized FLT3L (referred to as “hFLT3L” in FIG. 3) in both serum and bone marrow aspirate of SRG/hFLT3L/mFLT3 KO mice. “SRG/hFLT3L/mFLT3 KO” mice, also referred to as “SRG hFLT3L mFLT3 KO” mice, “SRG-hFLT3L/mFLT3 KO” mice, or “hFLT3L/mFLT3 KO” mice, refer to mice that comprise humanized Sirpa, Rag2 gene knock-out, Il2rg gene knock-out, humanized Flt31, and Flt3 KO.

FIG. 4A shows the gating strategy for pDCs and other dendritic cells.

FIG. 4B shows that SRG/hFLT3L/mFLT3 KO (referred to as “SRG hFLT3L mFLT3 KO” in FIG. 4B) mice had increased myeloid and plasmacytoid DCs in the blood and spleen upon hHSC engraftment.

FIG. 4C shows that SRG/hFLT3L/mFLT3 KO (referred to as “SRG hFLT3L mFLT3 KO” in FIG. 4C) mice had increased BDCA-3+ myeloid DCs in their blood and spleen upon hHSC engraftment.

FIG. 4D shows that SRG/hFLT3L/mFLT3 KO (referred to as “SRG hFLT3L mFLT3 KO” in FIG. 4D) mice had comparable engraftment to HSC donor-matched StRG mice but increased human myeloid and plasmacytoid DCs in the BM.

FIG. 4E shows that SRG/hFLT3L/mFLT3 KO (referred to as “SRG hFLT3L mFLT3 KO” in FIG. 4E) had increased human myeloid and plasmacytoid DCs in the thymus upon hHSC engraftment.

FIG. 4F shows that a significant loss of murine DCs (mouse CD45+/CD11c+/MHC Class II+) in engrafted SRG/hFLT3L/mFLT3 KO (referred to as “SRG-hFLT3L/mFLT3 KO” in FIG. 4F) mice.

FIG. 5A shows that human mDC and pDC populations were increased in the blood and spleen of hHSC-engrafted mice with humanized Flt31 and mouse Flt3 KO (i.e., hFLT3L/mFLT3 KO) relative to HSC donor-matched mice with just mFLT3 KO. “FLT3L^{m/m}FLT3−/−” mice refer to SRG mice that comprise homozygous mouse Flt31 and comprise mouse Flt3 knockout. “FLT3L^{h/h}mFLT3−/−” mice refer to SRG mice that comprise homozygous humanized Flt31 and comprise mouse Flt3 knockout.

FIG. 5B shows that human mDC and pDC populations were increased in the thymus of hHSC-engrafted mice with humanized Flt31 and mouse Flt3 KO (i.e., hFLT3L/mFLT3 KO) relative to HSC donor-matched mice with just mFLT3 KO. “FLT3L^{m/m}FLT3−/−” mice refer to SRG mice that comprise homozygous mouse Flt31 and comprise mouse Flt3 knockout. “FLT3L^{h/h}mFLT3−/−” mice refer to SRG mice that comprise homozygous humanized Flt31 and comprise mouse Flt3 knockout.

FIG. 6A shows that CD3+ T cells in the blood expressed significantly less PD-1 in hHSC-engrafted SRG/hFLT3L/mFLT3 KO (referred to as “SRG hFLT3L mFLT3 KO” in FIG. 6A) than in StRG mice; and that there is a higher percentage of CD4+ T cells than CD8+ T cells in hHSC-engrafted SRG/hFLT3L/mFLT3 KO mice.

FIG. 6B shows that greater CD4+ and CD8+ T cells in hHSC-engrafted SRG/hFLT3L/mFLT3 KO (referred to as “hFLT3L/mFLT3 KO” in FIG. 6B) were central memory T cells (Tcm) than in HSC donor-matched StRG.

FIG. 7A shows key transcriptional differences between DC populations in spleen/blood of hHSC-engrafted SRG/hFLT3L/mFLT3 KO mice and healthy human blood.

FIG. 7B shows a greater percentage of mDC, BDCA-3+ mDC, and pDC in hHSC-engrafted SRG/hFLT3L/mFLT3 KO (referred to as “SRG-hFLT3L/mFLT3 KO”) mice than in human PBMCs.

FIG. 8 shows the presence of human pDCs in various organs in SRG/hFLT3L/mFLT3 KO human immune system mice.

FIG. 9A shows levels of human IFNα or IFNβ produced by human pDCs isolated from the SRG/hFLT3L/mFLT3 KO human immune system mice stimulated with ODN2216 (CpG-DNA).

FIG. 9B shows that human pDCs in the SRG/hFLT3L/mFLT3 KO human immune system mice respond to ODN2216 CpG stimulation in vivo and produce human Type I IFN as measured by induction of Type I IFN signature genes.

### DETAILED DESCRIPTION

### General

The present disclosure relates to a genetically modified non-human animal (e.g., a rodent, such as a rat or a mouse) comprising: (i) a homozygous null mutation in Rag1 and/or Rag2 gene (e.g., a Rag1 and/or Rag2 gene knock-out); (ii) a homozygous null mutation in IL2rg gene (e.g., a IL2rg gene knock-out); (iii) a homozygous null mutation in the non-human animal FMS-like tyrosine kinase 3 (Flt3) gene at the non-human animal Flt3 gene locus, and (iv) a Flt3 ligand (Flt31) gene that comprises a non-human animal portion and a human portion operably linked to a Flt31 promoter. In some embodiments, provided herein is a genetically modified non-human animal (e.g., mouse) comprising: (i) a homozygous null mutation in Rag2 gene (e.g., a Rag2 gene knock-out); (ii) a homozygous null mutation in IL2rg gene (e.g., a IL2rg gene knock-out); (iii) a homozygous null mutation in the non-human animal FMS-like tyrosine kinase 3 (Flt3) gene, and (iv) a Flt3 ligand (Flt31) gene that comprises a non-human animal portion and a human portion operably linked to a Flt31 promoter.

In some embodiments, the genetically modified non-human animal expresses a human or humanized SIRPA protein encoded by a nucleic acid operably linked to a promoter, e.g., a Sirpa promoter. In some embodiments, the genetically modified non-human animal further expresses a human GM-CSF protein encoded by a nucleic acid operably linked to a promoter, e.g., a GM-CSF promoter, and/or a human IL3 protein encoded by a nucleic acid operably linked to a promoter, e.g., a IL3 promoter. In certain embodiments, at least one promoter operably linked to a nucleic acid that encodes a human or humanized protein is an endogenous non-human animal promoter. In other embodiments, the genetically modified animal expresses a human or humanized nucleic acid from the native human promoter and native regulatory elements. The skilled artisan will understand that the genetically modified animal includes genetically modified animals that express at least one human or humanized nucleic acid from any promoter. Examples of promoters useful in the invention include, but are not limited to, DNA pol II promoter, PGK promoter, ubiquitin promoter, albumin promoter, globin promoter, ovalbumin promoter, SV40 early promoter, the Rous sarcoma virus (RSV) promoter, retroviral LTR and lentiviral LTR. Promoter and enhancer expression systems useful in present disclosure also include inducible and/or tissue-specific expression systems.

Rodents, such as mice and rats with components of the human immune system (e.g., HIS mice and rats) hold great promise for studying the human immune system in vivo and for testing human vaccines and testing and developing drugs to treat human diseases and disorders. HIS mice are generated by transplanting a severely immunodeficient mouse strain (such as recombination-activating gene 2 (Rag2) knockout (KO) and interleukin 2 receptor gamma (IL2rg) KO mice) with human hematopoietic stem and progenitor cells (such as CD34+ HSCs). Compared to nonhuman primates, HIS mice have the advantages of a small animal model, i.e., they allow more versatile experimentation, are more accessible to the research community, and are ethically more acceptable than conducting experiments with human subjects. Most importantly, experimental findings derived from HIS mice can be more relevant and applicable to humans. Similar rat models that offer such advantages have also been described and are contemplated herein.

Although HIS mice develop human immune cells such as B and T cells, there is a need for HIS mouse models that support development of human dendritic cells, the primary antigen-presenting cells of the immune system that modulate T cell activation and antigen-specific responses. Fms tyrosine like 3 ligand (Flt31) is a critical cytokine for development of dendritic cells. However, it was found that humanization of Flt31 in a HIS mouse model (e.g., the huSIRP Rag2−/− IL-2Rγ−/− (SRG) VelociHum model) did not significantly increase human dendritic cell development. It has been reported that mice with deleted Flt3 have increased human DC development upon human hematopoietic stem cell (hHSC) engraftment. However, increased human DCs required repeated injection of large doses of human FLT3L (Li et al. (2016) Eur J Immunol 46:1291-1299).

The present disclosure provides a new genetically modified non-human animal (e.g., mouse) model that specifically deletes the non-human animal (e.g., mouse) receptor for Flt31 (Flt3 or FLK2 (fetal liver kinase 2)) in a HIS model (e.g., the huSIRP Rag2−/− IL-2Rγ−/− (SRG) VelociHum model) with humanized Flt31. The new model has receptor deletion with concomitant humanization of Flt31, such that Flt31 isoforms are expressed continuously at physiologically-relevant levels. In addition, the humanized Flt31 gene provided herein encodes a chimeric membrane-bound FLT3L that contains a human signal peptide and cytokine-like core domain, as well as a non-human animal (e.g., rodent such as a rat or a mouse) stalk, transmembrane domain, and cytosolic tail, which retains the endogenous functions that are associated with the stalk, transmembrane domain, and cytosolic tail. The Flt3 null, Flt31 humanized non-human animal (e.g., mouse) model showed increased human DCs in the spleen, blood and bone marrow. This increase was reported in myeloid DCs, a subset important for T cell activation/modulation, as well as plasmacytoid DCs, a subset that is the primary type I IFN producer in response to viral infection. Furthermore, a significant increase in BDCA-3+ DCs which are an important subset for cross-priming of CD8+ T cells to exogenously-derived antigens such as viral elements originating from engulfed infected cells was also observed. Without wishing to be bound by theory, deleting the non-human animal (e.g., mouse) receptor for Flt31 (Flt3 or FLK2 (fetal liver kinase 2)) ablates competition for the human FLT3L by non-human animal (e.g., mouse) DCs and thus potentiates human DC development in the HIS models with humanized Flt31. The present disclosure demonstrates that this non-human animal (e.g., mouse) cytokine receptor deletion allows for increased human dendritic cells (e.g., human myeloid and plasmacytoid dendritic cells) in the bone marrow and thymus at appreciably higher levels than those observed in StRG. This new model provides a useful tool to study human dendritic cell biology as well as a useful model for developing treatments for human dendritic cell diseases.

The genetically modified non-human animals provided herein find many uses in the art, including, for example, in modeling human autoimmune diseases and dendritic cell functions; in in vivo screens for agents that modulate dendritic cell function, e.g., in a healthy or a diseased state; in assessing therapeutic efficacy of a drug to stimulate a T cell response to a target cell, and/or in assessing therapeutic efficacy of a drug in treating an autoimmune disease. These and other objects, advantages, and features of the invention will become apparent to those persons skilled in the art upon reading the details of the compositions and methods as more fully described below.

### Definitions

The articles “a” and “an” are used herein to refer to one or to more than one (i.e., to at least one) of the grammatical object of the article. By way of example, “an element” means one element or more than one element.

The term “amino acid” is intended to embrace all molecules, whether natural or synthetic, which include both an amino functionality and an acid functionality and capable of being included in a polymer of naturally-occurring amino acids. Exemplary amino acids include naturally-occurring amino acids; analogs, derivatives and congeners thereof; amino acid analogs having variant side chains; and all stereoisomers of any of the foregoing.

A “coding region” of a gene includes the nucleotide residues of the coding strand of the gene and the nucleotides of the non-coding strand of the gene which are homologous with or complementary to, respectively, the coding region of an mRNA molecule which is produced by transcription of the gene. A “coding region” of a mRNA molecule also includes the nucleotide residues of the mRNA molecule which are matched with an anti-codon region of a transfer RNA molecule during translation of the mRNA molecule or which encode a stop codon. The coding region may thus include nucleotide residues comprising codons for amino acid residues which are not present in the mature protein encoded by the mRNA molecule (e.g., amino acid residues in a protein export signal sequence).

As used herein, the term “chimeric” refers to nucleic acids or proteins whose structures (i.e., nucleotide or amino acid sequences) include portions that are from different species. In some embodiments, the “chimeric” nucleic acids or proteins described herein include nucleotide or amino acid sequences that are from both a non-human source (e.g., a rodent, e.g., a mouse) and a human. In such embodiments, the “chimeric” nucleic acids or proteins can also be referred to as “humanized” nucleic acids or protein.

As used herein, the phrase “endogenous gene” or “endogenous gene segment” refers to a gene or gene segment found in a parent or reference organism prior to introduction of a disruption, deletion, replacement, alteration, or modification as described herein. In some embodiments, a reference organism is a wild-type organism. In some embodiments, a reference organism is an engineered organism. In some embodiments, a reference organism is a laboratory-bred organism (whether wild-type or engineered).

As used herein, the term “full coding sequence” refers to a coding nucleic acid sequence ranging from the start codon to the stop codon. The term “full-length polypeptide” refers to a polypeptide that comprises an amino acid sequence that is encoded by such a full coding sequence.

The term “humanized”, is used herein in accordance with its art-understood meaning to refer to nucleic acids or proteins whose structures (i.e., nucleotide or amino acid sequences) include portions that are from a non-human source, which are engineered to have a structure and function more similar to true human nucleic acids or proteins than the original source nucleic acids or proteins. For example, humanizing can involve selecting amino acid substitutions to make a non-human sequence more similar to a human sequence. Humanizing can also involve grafting at least a portion of a non-human protein into a human protein. To give but one example, in the case of a membrane receptor, a “humanized” gene may encode a polypeptide having an extracellular portion having an amino acid sequence as that of a human extracellular portion and the remaining sequence as that of a non-human (e.g., mouse) polypeptide. In some embodiments, a humanized gene comprises at least a portion of a DNA sequence of a human gene. In some embodiments, a humanized protein comprises a sequence having a portion that appears in a human protein. The term “human” is art recognized and refers to nucleic acids or proteins whose structures (i.e., nucleotide or amino acid sequences) are entirely from a human source.

As used herein, the term “locus” refers to a location on a chromosome that contains a set of related genetic elements (e.g., genes, gene segments, regulatory elements). A locus can be endogenous or non-endogenous. The term “endogenous locus” refers to a location on a chromosome at which a particular genetic element is naturally found. In some embodiments, an endogenous locus has a sequence found in nature. In some embodiments, an endogenous locus is a wild-type locus. In some embodiments, an endogenous locus is an engineered locus.

The phrase “non-human animal” as used herein refers to any vertebrate organism that is not a human. In some embodiments, a non-human animal is a cyclostome, a bony fish, a cartilaginous fish (e.g., a shark or a ray), an amphibian, a reptile, a mammal, and a bird. In some embodiments, a non-human mammal is a primate, a goat, a sheep, a pig, a dog, a cow, or a rodent. In some embodiments, a non-human animal is a rodent such as a rat or a mouse.

The phrase “operably linked”, as used herein, refers to a juxtaposition wherein the components described are in a relationship permitting them to function in their intended manner. A control sequence “operably linked” to a coding sequence is ligated in such a way that expression of the coding sequence is achieved under conditions compatible with the control sequences. “Operably linked” sequences include both expression control sequences that are contiguous with the gene of interest and expression control sequences that act in trans or at a distance to control the gene of interest. The term “expression control sequence” as used herein refers to polynucleotide sequences which are necessary to affect the expression and processing of coding sequences to which they are ligated. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (i.e., Kozak consensus sequence); sequences that enhance protein stability; and when desired, sequences that enhance protein secretion. The nature of such control sequences differs depending upon the host organism. For example, in prokaryotes, such control sequences generally include promoter, ribosomal binding site, and transcription termination sequence, while in eukaryotes, typically, such control sequences include promoters and transcription termination sequence. The term “control sequences” is intended to include components whose presence is essential for expression and processing, and can also include additional components whose presence is advantageous, for example, leader sequences and fusion partner sequences.

The terms “polynucleotide”, and “nucleic acid” are used interchangeably. They refer to a polymeric form of nucleotides of any length, either deoxyribonucleotides or ribonucleotides, or analogs thereof. Polynucleotides may have any three-dimensional structure, and may perform any function. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. A polynucleotide may be further modified, such as by conjugation with a labeling component. In all nucleic acid sequences provided herein, U nucleotides are interchangeable with T nucleotides.

The term “polypeptide”, as used herein, refers to any polymeric chain of amino acids. In some embodiments, a polypeptide has an amino acid sequence that occurs in nature. In some embodiments, a polypeptide has an amino acid sequence that does not occur in nature. In some embodiments, a polypeptide has an amino acid sequence that is engineered in that it is designed and/or produced through action of the hand of man.

The term “promoter” as used herein includes a DNA sequence operably linked to a nucleic acid sequence to be transcribed such as a nucleic acid sequence encoding a desired molecule. A promoter is generally positioned upstream of a nucleic acid sequence to be transcribed and provides a site for specific binding by RNA polymerase and other transcription factors. In specific embodiments, a promoter is generally positioned upstream of the nucleic acid sequence transcribed to produce the desired molecule, and provides a site for specific binding by RNA polymerase and other transcription factors. The phrase “endogenous promoter” refers to a promoter that is naturally associated, e.g., in a wild-type organism, with an endogenous gene.

The term “recombinant”, as used herein, is intended to refer to polypeptides (e.g., signal-regulatory proteins as described herein) that are designed, engineered, prepared, expressed, created or isolated by recombinant means, such as polypeptides expressed using a recombinant expression vector transfected into a host cell, polypeptides isolated from a recombinant, combinatorial human polypeptide library (Hoogenboom H. R., (1997) TIB Tech. 15:62-70; Azzazy H., and Highsmith W. E., (2002) Clin. Biochem. 35:425-445; Gavilondo J. V., and Larrick J. W. (2002) BioTechniques 29:128-145; Hoogenboom H., and Chames P. (2000) Immunology Today 21:371-378), antibodies isolated from an animal (e.g., a mouse) that is transgenic for human immunoglobulin genes (see e.g., Taylor, L. D., et al. (1992) Nucl. Acids Res. 20:6287-6295; Kellermann S-A., and Green L. L. (2002) Current Opinion in Biotechnology 13:593-597, Little M. et al (2000) Immunology Today 21:364-370) or polypeptides prepared, expressed, created or isolated by any other means that involves splicing selected sequence elements to one another. In some embodiments, one or more of such selected sequence elements is found in nature. In some embodiments, one or more of such selected sequence elements is designed in silico. In some embodiments, one or more such selected sequence elements results from mutagenesis (e.g., in vivo or in vitro) of a known sequence element, e.g., from a natural or synthetic source. For example, in some embodiments, a recombinant polypeptide is comprised of sequences found in the genome of a source organism of interest (e.g., human, mouse, etc.). In some embodiments, a recombinant polypeptide has an amino acid sequence that resulted from mutagenesis (e.g., in vitro or in vivo, for example in a non-human animal), so that the amino acid sequences of the recombinant polypeptides are sequences that, while originating from and related to polypeptides sequences, may not naturally exist within the genome of a non-human animal in vivo.

The term “replacement” is used herein to refer to a process through which a “replaced” nucleic acid sequence (e.g., a gene) found in a host locus (e.g., in a genome) is removed from that locus and a different, “replacement” nucleic acid is located in its place. In some embodiments, the replaced nucleic acid sequence and the replacement nucleic acid sequences are comparable to one another in that, for example, they are homologous to one another and/or contain corresponding elements (e.g., protein-coding elements, regulatory elements, etc.). In some embodiments, a replaced nucleic acid sequence includes one or more of a promoter, an enhancer, a splice donor site, a splice receiver site, an intron, an exon, an untranslated region (UTR); in some embodiments, a replacement nucleic acid sequence includes one or more coding sequences. In some embodiments, a replacement nucleic acid sequence is a homolog of the replaced nucleic acid sequence. In some embodiments, a replacement nucleic acid sequence is an ortholog of the replaced sequence. In some embodiments, a replacement nucleic acid sequence is or comprises a human nucleic acid sequence. In some embodiments, including where the replacement nucleic acid sequence is or comprises a human nucleic acid sequence, the replaced nucleic acid sequence is or comprises a rodent sequence (e.g., a mouse sequence). The nucleic acid sequence so placed may include one or more regulatory sequences that are part of source nucleic acid sequence used to obtain the sequence so placed (e.g., promoters, enhancers, 5′- or 3′-untranslated regions, etc.). For example, in various embodiments, the replacement is a substitution of an endogenous sequence with a heterologous sequence that results in the production of a gene product from the nucleic acid sequence so placed (comprising the heterologous sequence), but not expression of the endogenous sequence; the replacement is of an endogenous genomic sequence with a nucleic acid sequence that encodes a protein that has a similar function as a protein encoded by the endogenous sequence. In various embodiments, an endogenous gene or fragment thereof is replaced with a corresponding human gene or fragment thereof. A corresponding human gene or fragment thereof is a human gene or fragment that is an ortholog of, or is substantially similar or the same in structure and/or function, as the endogenous gene or fragment thereof that is replaced.

“Variant” as the term is used herein, includes a nucleic acid sequence or a peptide sequence that differs in sequence from a reference nucleic acid sequence or peptide sequence respectively, but retains essential biological properties of the reference molecule. Changes in the sequence of a nucleic acid variant may not alter the amino acid sequence of a peptide encoded by the reference nucleic acid, or may result in amino acid substitutions, additions, deletions, fusions and truncations. Changes in the sequence of peptide variants are typically limited or conservative, so that the sequences of the reference peptide and the variant are closely similar overall and, in many regions, identical. A variant and reference peptide can differ in amino acid sequence by one or more substitutions, additions, deletions in any combination. A variant of a nucleic acid or peptide can be a naturally occurring such as an allelic variant, or can be a variant that is not known to occur naturally. Non-naturally occurring variants of nucleic acids and peptides may be made by mutagenesis techniques or by direct synthesis.

The term “vector”, as used herein, refers to a nucleic acid molecule capable of transporting another nucleic acid to which it is associated. In some embodiment, vectors are capable of extra-chromosomal replication and/or expression of nucleic acids to which they are linked in a host cell such as a eukaryotic and/or prokaryotic cell. Vectors capable of directing the expression of operatively linked genes are referred to herein as “expression vectors.”

The term “wild-type”, as used herein, has its art-understood meaning that refers to an entity having a structure and/or activity as found in nature in a “normal” (as contrasted with mutant, diseased, altered, etc.) state or context. Those of ordinary skill in the art will appreciate that wild type genes and polypeptides often exist in multiple different forms (e.g., alleles).

### Genetically Modified Loci

In certain aspects provided herein are genetically modified non-human animals (e.g., mice or rats) comprising: (i) a homozygous null mutation in Rag1 and/or Rag2 gene (e.g., a Rag1 and/or Rag2 gene knock-out); (ii) a homozygous null mutation in IL2rg gene (e.g., a IL2rg gene knock-out); (iii) a homozygous null mutation in the non-human animal FMS-like tyrosine kinase 3 (Flt3) gene, and (iv) a Flt3 ligand (Flt31) gene that comprises a non-human animal portion and a human portion operably linked to a Flt31 promoter. In some embodiments, provided herein are genetically modified non-human animals (e.g., mice) comprising: (i) a homozygous null mutation in Rag2 gene (e.g., a Rag2 gene knock-out); (ii) a homozygous null mutation in IL2rg gene (e.g., a IL2rg gene knock-out); (iii) a homozygous null mutation in the non-human animal FMS-like tyrosine kinase 3 (Flt3) gene, and (iv) a Flt3 ligand (Flt31) gene that comprises a non-human animal portion and a human portion operably linked to a Flt31 promoter. In some embodiments, the genetically modified non-human animal expresses a human or humanized SIRPA protein encoded by a nucleic acid operably linked to a Sirpa promoter. In certain embodiments, the genetically modified non-human animal further expresses a human GM-CSF protein encoded by a nucleic acid operably linked to a GM-CSF promoter and/or a human IL3 protein encoded by a nucleic acid operably linked to an IL3 promoter. In certain embodiments, at least one promoter operably linked to a nucleic acid that encodes a human or humanized protein is an endogenous non-human animal promoter. In some embodiments, the genetically modified non-human animal comprises engraftment of human hematopoietic stem cells (HSC).

### Flt3 Knockout

In certain aspects, genetically modified non-human animals (e.g., mice or rats) provided herein comprise a homozygous null mutation in the non-human animal FMS-like tyrosine kinase 3 (Flt3) gene.

Flt3 gene encodes a class III receptor tyrosine kinase that regulates hematopoiesis. This receptor is activated by binding of the fms-related tyrosine kinase 3 ligand to the extracellular domain, which induces homodimer formation in the plasma membrane leading to autophosphorylation of the receptor. The activated receptor kinase subsequently phosphorylates and activates multiple cytoplasmic effector molecules in pathways involved in apoptosis, proliferation, and differentiation of hematopoietic cells in bone marrow. Mutations that result in the constitutive activation of this receptor result in acute myeloid leukemia and acute lymphoblastic leukemia.

In certain aspects, the genetically modified non-human animal comprises a homozygous null mutation in the non-human animal FMS-like tyrosine kinase 3 (Flt3) gene. A null mutation comprises a deletion, an insertion, and/or a substitution in a gene which leads to no functional gene product (e.g., complete absence of the gene product (protein, RNA) at the molecular level, or the expression of a non-functional gene product). In the present disclosure, a null mutation has the same meaning and is used interchangeably with an inactivating mutation. A homozygous null mutation refers to having a null mutation in all alleles. For example, a homozygous null mutation in mouse or rat Flt3 gene refers to having a null mutation in two alleles (i.e., two null alleles) for mouse or rat Flt3 gene. A gene with a homozygous null mutation is also referred to as a gene knockout or a gene deficient/deficiency. For example, a homozygous null mutation in Flt3 is also referred to as a Flt3 knockout or a Flt3-deficiency. Thus, the null mutation in the non-human animal Flt3 gene comprises a deletion, an insertion, and/or a substitution in the non-human animal Flt3 gene. In some instances, the endogenous non-human animal Flt3 locus comprises a null mutation, and hence, a null allele. A null allele is a mutant copy of a gene that completely lacks that gene's normal function. This can be the result of the complete absence of the gene product (protein, RNA) at the molecular level, or the expression of a non-functional gene product. At the phenotypic level, a null allele includes a deletion of the entire locus.

In some embodiments, the null mutation is a deletion of the full Flt3 endogenous coding sequence. In some embodiments, the non-human animals provided herein do not express Flt3 protein.

In some embodiments, the homozygous null mutation in the non-human animal Flt3 gene comprises the same null mutation for all the alleles. In some embodiments, the homozygous null mutation in the non-human animal Flt3 gene comprises different null mutations for different alleles.

Mouse Flt3 is located on Chromosome 5, GRCm38, NC_000071.6 (147330741-147400489), and the mouse Flt3 coding sequence may be found at Genbank Accession No. NM_010229.2. The mouse Flt3 locus includes 24 exons, with exons 1-24 being coding exons. As such, in some embodiments, the genetically modified animals provided herein are mice, and one or more of exons 1-24 of the mouse Flt3 gene are deleted or mutated in the genetically modified mice. In some instances, other aspects of the genomic locus of the mouse Flt3 gene, e.g., introns, 3′ and/or 5′ untranslated sequence (UTRs) are also deleted or mutated. In some instances, the whole regions of the mouse Flt3 genomic locus are deleted. In some embodiments, the whole genomic region from the start codon to the stop codon of the mouse Flt3 gene is deleted. For example, the genetically modified mice may comprise a deletion of 69.1 kb mouse genomic sequence of the mouse Flt3 gene, with the sequence from the start codon ATG to the stop codon, deleted on mouse chromosome 5 G3, between coordinates chr5: 147331171-147400265 (GRCm38 assembly) as illustrated in Example 1.

The deleted, modified or altered Flt3 gene at the endogenous Flt3 locus can be detected using a variety of methods including, for example, PCR, Western blot, Southern blot, restriction fragment length polymorphism (RFLP), or a gain or loss of allele assay. In some embodiments, the non-human animal is homozygous for the deletion or null mutation of the endogenous Flt3 gene.

In some embodiments, the non-human animal (e.g., mouse or rat) comprising a homozygous null mutation in Flt3 gene, i.e., the Flt3 deficient non-human animal, is an immunocompromised animal. For example, the Flt3 deficient non-human animal (e.g., mouse or rat) may include at least one null allele for the Rag2 gene (“recombination activating gene 2”, wherein the coding sequence for the mouse gene may be found at Genbank Accession No. NM_009020.3). In some embodiments, the Flt3 deficient non-human animal (e.g., mouse or rat) includes two null alleles for Rag2. In other words, the Flt3 deficient non-human animal (e.g., mouse) is homozygous null for Rag2. In other embodiments, Flt3 deficient non-human animal (e.g., mouse or rat) includes one or two null alleles for Rag1 gene. In some embodiments, the Flt3 deficient non-human animal (e.g., mouse or rat) is homozygous null for Rag1. In some embodiments, the Flt3 deficient non-human animal (e.g., mouse or rat) includes (i) one or two null alleles for Rag1 gene; and (ii) one or two null alleles for Rag2 gene. In some embodiments, the Flt3 deficient non-human animal (e.g., mouse or rat) is homozygous null for both Rag1 and Rag2. In some embodiments, the Flt3 deficient non-human animal is an immunocompromised mouse comprising two null alleles (i.e., homozygous null) for Rag2. In some embodiments, the Flt3 deficient non-human animal is an immunocompromised rat comprising two null alleles (i.e., homozygous null) for Rag1 and two null alleles (i.e., homozygous null) for Rag2. As another example, the Flt3 deficient non-human animal (e.g., mouse or rat) includes at least one null allele for the IL2rg gene (“interleukin 2 receptor, gamma”, also known as the common gamma chain, or γC, wherein the coding sequence for the mouse gene may be found at Genbank Accession No. NM 013563.3). In some embodiments, the Flt3 deficient non-human animal (e.g., mouse or rat) includes two null alleles for IL2rg. In other words, the Flt3 deficient non-human animal (e.g., mouse or rat) is homozygous null for IL2rg, i.e., it is IL2rg^{-/-} (or IL2rg^{γ/-} where the IL2rg gene is located on the X chromosome as in mouse or rat). In some embodiments, the Flt3 deficient non-human animal (e.g., mouse or rat) includes a null allele for both Rag2 and IL2rg, i.e., it is Rag2^{-/-} IL2rg^{-/-} (or Rag2^{-/-} IL2rg^{γ/-} where the IL2rg gene is located on the X chromosome as in mouse or rat). In some embodiments, the Flt3 deficient non-human animal (e.g., mouse or rat) includes a null allele for both Rag1 and IL2rg. In some embodiments, the Flt3 deficient non-human animal (e.g., mouse or rat) includes a null allele for Rag1, Rag2, and IL2rg. In some embodiments, the Flt3 deficient non-human animal is an immunocompromised mouse comprising two null alleles (i.e., homozygous null) for Rag2 and two null alleles (i.e., homozygous null) for IL2rg (or one null allele for male mice). In some embodiments, the Flt3 deficient non-human animal is an immunocompromised rat comprising two null alleles (i.e., homozygous null) for Rag1, two null alleles (i.e., homozygous null) for Rag2, and two null alleles (i.e., homozygous null) for IL2rg (or one null allele for male rat). Other genetic modifications are also contemplated. For example, the Flt3 deficient non-human animal (e.g., mouse or rat) may include modifications in other genes associated with the development and/or function of dendritic cells and the immune system, e.g., the replacement of one or more other non-human animal genes with nucleic acid sequence(s) encoding human or humanized polypeptides. Such genes include but are not limited to, e.g., FLT3L, SIRPA, GM-CSF, and/or IL-3. Additionally or alternatively, the Flt3 deficient non-human animal (e.g., mouse or rat) may include modifications in genes associated with the development and/or function of other cells and tissues, e.g., genes associated with human disorders or disease, or genes that, when modified in a non-human animal, e.g., mice, provide for models of human disorders and disease. Introduction of other genetic modifications may be accomplished by either ES cell modification and/or breeding. For example, the Flt3 deficient (and, optionally, Rag2 and IL2rg deficient) non-human animal (e.g., mouse or rat) may be bred with a non-human animal that comprises one or more other genetic modifications, including but not limited to, e.g., a modification in SIRPA, GM-CSF, and/or IL-3 gene. In some embodiments, all genetic modifications are bred to homozygous in the genetically modified animal described herein.

### Humanized Flt3l Loci

In certain aspects, the genetically modified non-human animals provided herein further express a chimeric Flt31 protein encoded by a Flt3 ligand (Flt31) gene that comprises a rodent portion and a human portion operably linked to a Flt31 promoter.

Dendritic cells (DCs) provide the key link between innate and adaptive immunity by recognizing pathogens and priming pathogen-specific immune responses. FLT3LG (also known as FL, FLG3L, or FLT3L) controls the development of DCs and is particularly important for plasmacytoid DCs and CD8-positive classical DCs and their CD103-positive tissue counterparts.

In some embodiments, the genetically modified non-human animals (e.g., rodents, such as rats or mice) provided herein comprises a Flt3 ligand (Flt31) gene that comprises a non-human animal (e.g., a rodent, such as a rat or a mouse) portion and a human portion operably linked to a Flt31 promoter.

In some embodiments, the non-human animal (e.g., a rodent, such as a rat or a mouse) portion of the Flt31 gene comprises exon 1, a non-coding portion of exon 2, and exons 7-9 each having a sequence at least 50% (e.g., 50%, 55%, 60%, 65%, 70%, 75%, 80%, 85%, 90%, 91%, 92%, 93%, 94%, 95%, 96%, 97%, 98%, 99% or 100%) identical to a non-coding portion of exon 2, and exons 7-9 of a non-human animal (e.g., a rodent, such as a rat or a mouse) Flt31 gene.

The genomic locus encoding the wild-type mouse Flt31 protein may be found in the mouse genome at Chromosome 7; GRCm39, NC_000073.7 (44780607-44785914). Polypeptide sequences for wild-type mouse Flt31 and the nucleic acid sequences that encode wild-type mouse Flt31 may be found at Genbank Accession Nos. NP_001389760.1 and NM_001402831.1; NP_001389761.1 and NM_001402832.1; NP_001389762.1 and NM_001402833.1; NP 001389763.1 and NM_001402834.1; NP_001389764.1 and NM_001402835.1; NP 001389765.1 and NM_001402836.1; NP_001389766.1 and NM_001402837.1; NP 038548.3 and NM_013520.4. In some embodiments, the membrane bound mouse Flt3 ligand polypeptide sequence used in the present disclosure is obtained from NCBI Reference Sequence: NP_038548.3, Uniprot: A9QW46; and is encoded by NM_013520.4.

In some embodiments, wherein the exon 1, the non-coding portion of exon 2, and the exons downstream of exon 6 (e.g., exons 7-9) of the non-human animal (e.g., a rodent, such as a rat or a mouse) Flt3L gene are at least 50% (e.g., 50%, 55%, 60%, 65%, 70%, 75%, 80%, 85%, 90%, 91%, 92%, 93%, 94%, 95%, 96%, 97%, 98%, 99% or 100%) identical to the corresponding exon 1, non-coding portion of exon 2, and exons 7-9 of a mouse Flt31 gene shown in Table 1A.

TABLE 1A

| Mouse Flt31 | |
|---|---|
| coding sequence | |
| (NM_013520.4) | Nucleotide Sequence |
| exon 1 | CTTTCACTTTCGGTCTTTGG |
| | CTGTCATCCAGCTTTGGCTT |
| | TCCACACCCAGCTGGGGCAA |
| | GCCTG (SEQ ID NO: 1) |
| non-coding | AACCTGTCACAGGCATGAGG |
| portion of | GGTCCCCGGCAGAG (SEQ |
| exon 2 | ID NO: 2) |
| exon 7 | ACTCCTCCACCCTGCTGCCC |
| | CCAAGGAGTCCCATAGCCCT |
| | AGAAGCCACGGAGCTCCCAG |
| | AGCCTCGGCCCAGGCAGCTG |
| | TTGCTCCTGCTGCTGCTGCT |
| | GCTGCCTCTCACACTGGTGC |
| | TGCTGGCAGCCGCCTGGGGC |
| | CTTCGCTGGCAAAGGGCAAG |
| | AAGGAGGGGGGAGCTCCACC |
| | CTGGG (SEQ ID NO: 3) |
| exon 8 | GTGCCCCTCCCCTCCCATCC |
| | CTAGGATGCGAGCCTTGTGC |
| | ATCGTTGACTCAGCCAGGGT |
| | CTTATCTCG |
| | (SEQ ID NO: 4) |
| exon 9 | AGTTGGGAACCAAAACAAGG |
| | AACAAGCTAGGCAAGTGCTG |
| | TGCTGAGTTACATCCCCAGC |
| | CCAGAGGACACACTGTCTGG |
| | GTATGGCGATGGACACTGTA |
| | ATTCCAGTGCTTCTGGATTG |
| | GACATGCTGAAACTGGAGGA |
| | TACTGACTTTAAGAAAAACA |
| | GAAAGGAAGAAGGGGGGTGC |
| | GTTGTAGCCCTGTGTGGTTG |
| | CTTTTGTCACCTCCACCCCT |
| | TAAAATAAAAGCAACTGCTG |
| | GGTA (SEQ ID NO: 5) |

In some embodiments, the human portion of the humanized Flt31 gene comprises a signal peptide-coding portion of exon 2, and exons 3-6 each having a sequence at least 50% (e.g., 50%, 55%, 60%, 65%, 70%, 75%, 80%, 85%, 90%, 91%, 92%, 93%, 94%, 95%, 96%, 97%, 98%, 99% or 100%) identical to a signal peptide-coding portion of exon 2, and exons 3-6 that appear in a human FLT3L gene.

The genomic locus encoding the wild-type human FLT3L protein may be found in the human genome at Chromosome 19; GRCh38.p14 NC_000019.10 (49474215-49486231). Polypeptide sequences for wild-type human FLT3L and the nucleic acid sequences that encode wild-type human FLT3L may be found at Genbank Accession Nos. NP_001191431.1 and NM_001204502.2 (isoform 1 and transcript variant 1); NP_001191432.1 and NM_001204503.2 (isoform 1 and transcript variant 2); NP_001265566.1 and NM_001278637.2 (isoform 2 and transcript variant 4); NP_001265567.1 and NM_001278638.2 (isoform 2 and transcript variant 5); and NP_001450.2 and NM_001459.4 (isoform 1 and transcript variant 3). In some embodiments, the membrane bound human FLT3 ligand polypeptide sequence used in the present disclosure is obtained from NCBI Reference Sequence: NP_001191431.1, Uniprot: P49771-1; and is encoded by NM_001204502.2.

In some embodiments, the signal peptide-coding portion of exon 2, and exons 3-6 of the human FLT3L gene are at least 50% (e.g., 50%, 55%, 60%, 65%, 70%, 75%, 80%, 85%, 90%, 91%, 92%, 93%, 94%, 95%, 96%, 97%, 98%, 99% or 100%) identical to the corresponding signal peptide-coding portion of exon 2, and exons 3-6 of a human FLT3L gene shown in Table 1B.

TABLE 1B

| Human FLT3L | |
|---|---|
| coding sequence | |
| (NM_001204502.2) | Nucleotide Sequence |
| signal peptide- | ATGACAGTGCTGGCGCCAGC |
| coding portion | CTGGAGCCCAACA |
| of exon 2 | (SEQ ID NO: 6) |
| exon 3 | ACCTATCTCCTCCTGCTGCT |
| | GCTGCTGAGCTCGGGACTCA |
| | GTGGGACCCAGGACTGCTCC |
| | TTCCAACACAGCCCCATCTC |
| | CTCCGACTTCGCTGTCAAAA |
| | TCCGTGAGCTG |
| | (SEQ ID NO: 7) |
| exon 4 | TCTGACTACCTGCTTCAAGA |
| | TTACCCAGTCACCGTGGCCT |
| | CCAACCTGCAGGAC |
| | (SEQ ID NO: 8) |
| exon 5 | GAGGAGCTCTGCGGGGGCCT |
| | CTGGCGGCTGGTCCTGGCAC |
| | AGCGCTGGATGGAGCGGCTC |
| | AAGACTGTCGCTGGGTCCAA |
| | GATGCAAGGCTTGCTGGAGC |
| | GCGTGAACACGGAGATACAC |
| | TTTGTCACCAAATGTGCCTT |
| | TCAG (SEQ ID NO:9) |
| exon 6 | CCCCCCCCCAGCTGTCTTCG |
| | CTTCGTCCAGACCAACATCT |
| | CCCGCCTCCTGCAGGAGACC |
| | TCCGAGCAGCTGGTGGCGCT |
| | GAAGCCCTGGATCACTCGCC |
| | AGAACTTCTCCCGGTGCCTG |
| | GAGCTGCAGTGTCAGCCCG |
| | (SEQ ID NO: 10) |

In some embodiments, the Flt31 gene comprises non-human animal (e.g., a rodent, such as a rat or a mouse) exon 1, rodent non-coding portion of exon 2, human signal peptide-coding portion of exon 2, human exons 3-6, and non-human animal (e.g., a rodent, such as a rat or a mouse) exons 7-9. In some instances, the Flt31 gene also includes aspects of the genomic locus of the human FLT3L, e.g., introns, 3′ and/or 5′ untranslated sequence (UTRs). In some instances, the Flt31 gene also includes aspects of the genomic locus of the non-human animal (e.g., a rodent, such as a rat or a mouse) Flt31, e.g., introns, 3′ and/or 5′ untranslated sequence (UTRs).

In some embodiments, the Flt31 gene encodes a chimeric membrane bound Flt31 comprising a signal peptide and cytokine-like core domain of a human FLT3L polypeptide, and a C-terminal portion of a rodent Flt31 polypeptide. The “cytokine-like core domain” and “cytokine domain” refer to the same portion of the human FLT3L polypeptide and are used interchangeably in the present disclosure.

An exemplary chimeric membrane bound Flt31 sequence is set forth in Table 2. The signal peptide of a human FLT3L polypeptide is underlined, the cytokine-like core domain of a human FLT3L polypeptide is in bold, and C-terminal portion of a mouse Flt31 polypeptide is italicized.

TABLE 2
Humanized Flt31 Protein (SEQ ID NO: 11)

MTVLAPAWSP TTYLLLLLLL SSGLSGTQDC
SFQHSPISSD FAVKIRELSD YLLQDYPVTV
ASNLQDEELC GGLWRLVLAQ RWMERLKTVA
GSKMQGLLER VNTEIHFVTK CAFQPPPSCL
RFVQTNISRL LQETSEQLVA LKPWITRQNF
SRCLELQCQP DSSTLLPPRS PIALEATELP
EPRPRQLLLL LLLLLPLTLV LLAAAWGLRW
QRARRRGELH PGVPLPSHP

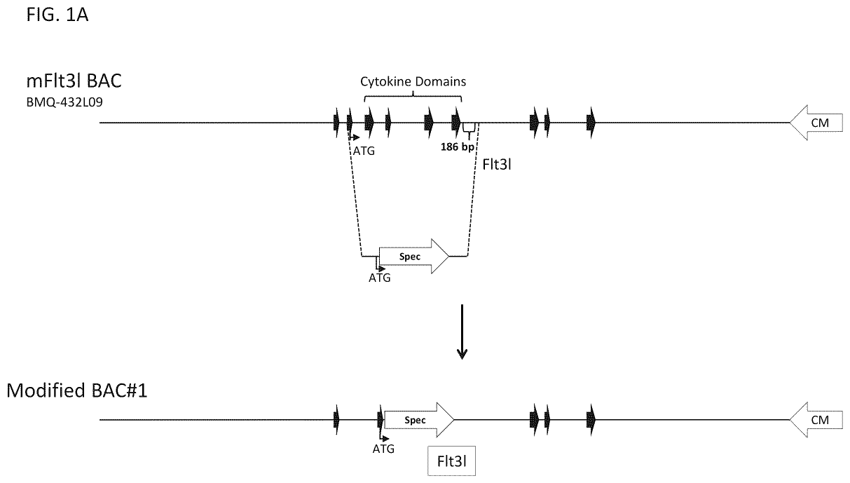

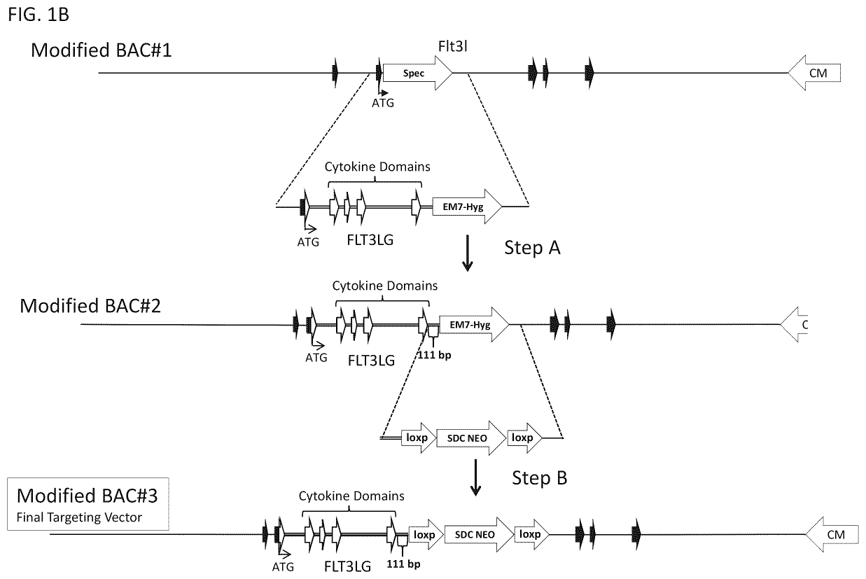

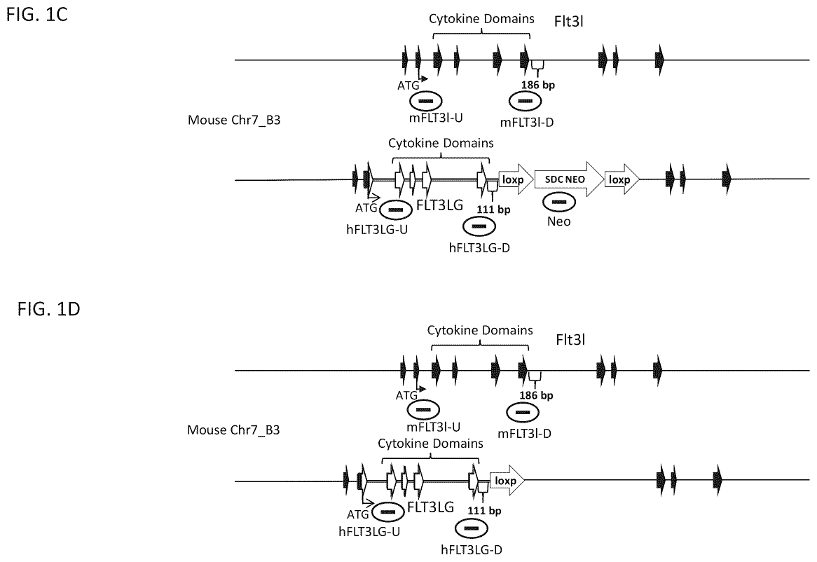

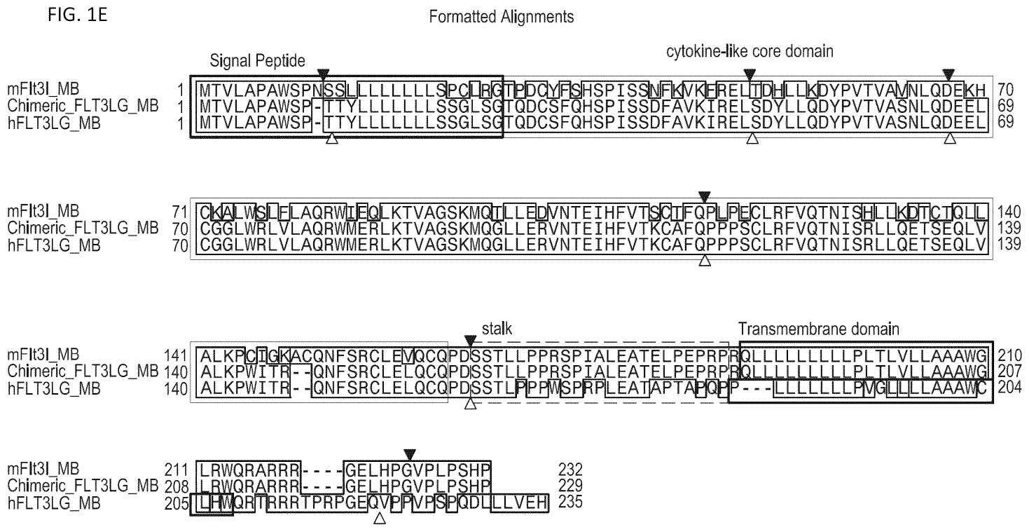

In some embodiments, the C-terminal portion of the rodent Flt31 polypeptide comprises a rodent stalk region, a rodent transmembrane domain, and a rodent cytoplasmic tail of a rodent Flt31 polypeptide, as shown in FIG. 1E. In some embodiments, the C-terminal portion of the rodent Flt31 polypeptide comprises amino acids corresponding to residues 165-232 of a mouse Flt31 polypeptide (e.g., NP_038548.3). In some embodiments, the C-terminal portion of the rodent Flt31 polypeptide has an amino acid sequence that is at least 50% (e.g., 50%, 55%, 60%, 65%, 70%, 75%, 80%, 85%, 90%, 91%, 92%, 93%, 94%, 95%, 96%, 97%, 98%, 99% or 100%) identical to a C-terminal portion of a rodent Flt31 polypeptide shown in Table 2.

In some embodiments, the signal peptide of the human FLT3L polypeptide comprises amino acids corresponding to residues 1-26 of a human FLT3L polypeptide (e.g., NP_001191431.1). In some embodiments, the signal peptide of the human FLT3L polypeptide comprises an amino acid sequence that is at least 50% (e.g., 50%, 55%, 60%, 65%, 70%, 75%, 80%, 85%, 90%, 91%, 92%, 93%, 94%, 95%, 96%, 97%, 98%, 99% or 100%) identical to a corresponding amino acid sequence of a signal peptide of a human FLT3L polypeptide that appears in Table 2.

In some embodiments, the cytokine-like core domain of the human FLT3L polypeptide comprises amino acids corresponding to residues 27-159 of a human FLT3L polypeptide (e.g., NP_001191431.1). In some embodiments, the cytokine-like core domain of the human FLT3L polypeptide comprises an amino acid sequence that is at least 50% (e.g., 50%, 55%, 60%, 65%, 70%, 75%, 80%, 85%, 90%, 91%, 92%, 93%, 94%, 95%, 96%, 97%, 98%, 99% or 100%) identical to a corresponding amino acid sequence of cytokine-like core domains of a human FLT3L polypeptide that appears in Table 2.

In some embodiments, the non-human animals provided herein express humanized Flt31 proteins resulting from a genetic modification of an endogenous locus of the non-human animal that encodes a Flt31 protein. Suitable examples described herein include rodents, in particular, mice.

Mouse and human Flt3 ligands exist as multiple isoforms, both membrane bound and soluble forms, which are believed to arise from alternative splicing (Lyman et al. (1995) Oncogene 10:149-157; McClanahan et al. (1996) Blood 88(9):3371-3382; Lyman et al. (1995) Oncogene 11:1165-1172; Lyman and Jackbsen (1998) Blood 91(4) 1101-1134, the content of each of which is incorporated by reference herein in its entirety). The soluble isoform can also arise from proteolytic cleavage (resulting from cleavage by TNFalpha converting enzyme or TACE) (Kazi et al. (2019) Physiol. Rev. 99:1433-1466, the content of which is incorporated by reference herein in its entirety). In some embodiments, the rodent expresses one or more soluble and/or membrane-bound forms of the Flt31 polypeptide. In some embodiments, the rodent expresses both soluble and membrane-bound forms of the Flt31 polypeptide. In some embodiments, the soluble and membrane-bound forms of the Flt31 polypeptide comprise a signal peptide and a cytokine-like core domain of a human FLT3L polypeptide. All known soluble isoforms of human and mouse Flt3 ligand comprise a receptor binding domain (cytokine-like core domain), generally encoded by exons 3-6 of the gene.

In some embodiments, the genetically modified rodent is heterozygous for the Flt3 ligand (Flt31) gene that comprises a non-human animals (e.g., rodents, such as rats or mice) portion and a human portion. In some embodiments, the genetically modified rodent is homozygous for the Flt3 ligand (Flt31) gene that comprises a non-human animals (e.g., rodents, such as rats or mice) portion and a human portion.

In some embodiments, the non-human animal (e.g., rodent, such as rat or mouse) portion of the Flt31 gene is an endogenous non-human animal (e.g., rodent, such as rat or mouse) portion of the Flt31 gene; the rodent Flt31 gene is an endogenous non-human animal (e.g., rodent, such as rat or mouse) gene; and/or the rodent Flt31 polypeptide is an endogenous non-human animal (e.g., rodent, such as rat or mouse) Flt31 polypeptide.

In some embodiments, the non-human animals (e.g., rodents, such as rats or mice) and human portions are operably linked to a Flt31 promoter. In some embodiments, the Flt31 promoter is a non-human animal (e.g., rodent, such as rat or mouse) promoter. In some embodiments, the Flt31 promoter is an endogenous non-human animal (e.g., rodent, such as rat or mouse) promoter. In some embodiments, the Flt31 promoter is at the endogenous non-human animal (e.g., rodent, such as rat or mouse) gene locus.

A genetically modified non-human animal, in some embodiments, comprises genetic material from a heterologous species (e.g., a human), wherein the non-human Flt31 gene encodes a Flt31 protein that comprises the encoded portion of the genetic material from the heterologous species. In some embodiments, a humanized Flt31 gene of the present disclosure comprises genomic DNA of a heterologous species that corresponds to the signal peptide and the cytokine-like core domain of a Flt31 protein. Non-human animals, embryos, cells and targeting constructs for making non-human animals, non-human embryos, and cells containing said humanized Flt31 gene are also provided.

In some embodiments, an endogenous non-human (e.g., rodent) Flt31 gene is deleted. In some embodiments, an endogenous non-human (e.g., rodent) Flt31 gene is altered, wherein a portion of the endogenous non-human (e.g., rodent) Flt31 gene is replaced with a heterologous sequence (e.g., a human FLT3L sequence in whole or in part). In some embodiments, all or substantially all of an endogenous non-human (e.g., rodent) Flt31 gene is replaced with a heterologous gene (e.g., a human FLT3L gene). In some embodiments, a portion of a heterologous FLT3L gene is inserted into an endogenous non-human Flt31 gene at an endogenous non-human (e.g., rodent) Flt31 locus. In some embodiments, the heterologous gene is a human gene. In some embodiments, the modification or humanization is made to one of the two copies of the endogenous non-human (e.g., rodent) Flt31 gene, giving rise to a non-human animal which is heterozygous with respect to the humanized Flt31 gene. In other embodiments, a non-human animal is provided that is homozygous for a humanized Flt31 gene. In some embodiments, the portion that encodes the signal peptide and cytokine-like core domain of the endogenous non-human (e.g., rodent) Flt31 gene is replaced with the corresponding heterologous sequence (e.g., a human FLT3L sequence), while the portion that encodes the stalk, transmembrane domain, and cytosolic tail remain endogenous non-human (e.g., rodent) Flt31 sequence. In such embodiments, the non-human (e.g., rodent) TACE cleavage is preserved.

A non-human animal of the present disclosure contains a human FLT3L gene in whole or in part at an endogenous non-human Flt31 locus. Thus, such non-human animals can be described as having a heterologous FLT3L gene. The replaced, inserted or modified Flt31 gene at the endogenous non-human (e.g., rodent) Flt31 locus can be detected using a variety of methods including, for example, PCR, Western blot, Southern blot, restriction fragment length polymorphism (RFLP), or a gain or loss of allele assay. In some embodiments, the non-human animal is heterozygous with respect to the humanized Flt31 gene.

Compositions and methods for making non-human animals that express a humanized Flt31 protein, including specific polymorphic forms or allelic variants (e.g., single amino acid differences), are provided. In some embodiments, compositions and methods for making non-human animals that express such proteins from an endogenous promoter and an endogenous regulatory sequence are provided. The methods include inserting the genetic material encoding a human FLT3L protein in whole or in part at a precise location in the genome of a non-human animal that corresponds to an endogenous non-human (e.g., rodent) Flt31 gene thereby creating a humanized Flt31 gene that expresses a Flt31 protein that is human in whole or in part. In some embodiments, the methods include inserting genomic DNA corresponding to a signal peptide-coding portion of exon 2 starting from the start codon “ATG”, exons 3-6 of a human FLT3L gene into an endogenous non-human (e.g., rodent) Flt31 gene of the non-human animal thereby creating a humanized gene that encodes a Flt31 protein that contains a human portion containing amino acids encoded by the inserted exons.

In various embodiments, a humanized Flt31 gene approach employs a relatively minimal modification of the endogenous gene and results in natural Flt31-mediated signal transduction in the non-human animal. Thus, in such embodiments, the Flt31 gene modification does not affect other surrounding genes. Further, in various embodiments, the modification does not affect the alternative splicing pattern or post-translational cleavage of Flt31.

In addition to mice having humanized Flt31 genes as described herein, also provided herein are other genetically modified non-human animals that comprise humanized Flt31 genes. In some embodiments, such non-human animals comprise a humanized Flt31 gene operably linked to an endogenous non-human (e.g., rodent) Flt31 promoter. In some embodiments, such non-human animals express a humanized Flt31 protein from an endogenous locus, wherein the humanized Flt31 protein comprises at least amino acid residues 27-160, for examples, amino acid residues 27-161, 27-162, 27-163, 27-164, 27-165, 1-160, 1-161, 1-162, 1-163, 1-164, or 1-165 of a human FLT3L protein.

### Immunodeficient Non-Human Animals

As explained above, genetically modified non-human animals comprising Flt3 deficiency and Flt31 humanization described herein are also immunodeficient because they comprise a deficiency in Rag1 and/or Rag2, and Il2rg genes. Rag1, Rag2, and Il2rg are essential components of the adaptive immune system. When one or more of these genes are mutated in animals, T-cells and B-cells do not mature, and the animals are severely compromised. When these animals are challenged with xenotransplanted cells, they are unable to mount an immune response to the foreign cells.

V(D)J recombination-activating protein 1 (also known as RAG1, recombination-activating 1, recombination activating gene 1, and recombination activating protein 1) is encoded by the Rag1 gene (also known as recombination activating 1). RAG1 is a catalytic component of the RAG complex, a multiprotein complex that mediates the DNA cleavage phase during V(D)J recombination. V(D)J recombination assembles a diverse repertoire of immunoglobulin and T-cell receptor genes in developing B and T-lymphocytes through rearrangement of different V (variable), in some cases D (diversity), and J (joining) gene segments. In the RAG complex, RAG1 mediates the DNA-binding to the conserved recombination signal sequences (RSS) and catalyzes the DNA cleavage activities by introducing a double-strand break between the RSS and the adjacent coding segment. RAG2 is not a catalytic component but is required for all known catalytic activities. RAG1 and RAG2 are essential to the generation of mature B cells and T cells, two types of lymphocytes that are crucial components of the adaptive immune system.

Mouse Rag1 maps to 2 E2; 2 53.88 cM on chromosome 2 (NCBI RefSeq Gene ID 19373; Assembly GRCm39 (GCF_000001635.27); location NC_000068.8 (101468597..101479877, complement). Reference to the mouse Rag1 gene includes the canonical, wild type form as well as all allelic forms and isoforms. The canonical, wild type mouse RAG1 protein has been assigned UniProt accession number P15919 and NCBI Accession No. NP_033045.2. Reference to mouse RAG1 proteins includes wild type forms as well as all allelic forms and isoforms. An mRNA (cDNA) encoding the canonical isoform is assigned NCBI Accession No. NM_009019.2. Reference to the mouse Rag1 mRNA (cDNA) and coding sequence includes the canonical, wild type forms as well as all allelic forms and isoforms.

Rat Rag1 maps to 3q31 on chromosome 3 (NCBI RefSeq Gene ID 84600; Assembly mRatBN7.2 (GCF_015227675.2); location NC_051338.1 (87917061..87928158, complement). Reference to the rat Rag1 gene includes the canonical, wild type form as well as all allelic forms and isoforms. The canonical, wild type rat RAG1 protein has been assigned UniProt accession number G3V6K9 and NCBI Accession No. NP_445920.1. Reference to rat RAG1 proteins includes canonical, wild type forms as well as all allelic forms and isoforms. An mRNA (cDNA) encoding the canonical isoform is assigned NCBI Accession No. NM_053468.1. Reference to the rat Rag1 mRNA (cDNA) and coding sequence includes the canonical, wild type forms as well as all allelic forms and isoforms.

An inactivated endogenous Rag1 gene is a Rag1 gene that does not produce a RAG1 protein or does not produce a functional RAG1 protein. The non-human animal (or cell or genome) can comprise the inactivated Rag1 gene in its germline. The non-human animal (or cell or genome) can be homozygous for an inactivating mutation in the Rag1 gene. As one example, an inactivated endogenous Rag1 gene can comprise an insertion, a deletion, or one or more point mutations in the endogenous Rag1 gene resulting in loss of expression of functional RAG1 protein. Some inactivated endogenous Rag1 genes can comprise a deletion or disruption of all of the endogenous Rag1 gene or can comprise a deletion or disruption of a fragment of (i.e., a part of or portion of) the endogenous Rag1 gene. For example, some, most, or all of the coding sequence in the endogenous Rag1 gene can be deleted or disrupted. In one example, a 5′ fragment of the Rag1 gene can be deleted or disrupted (e.g., including the start codon). As one example, an inactivated endogenous Rag1 gene can be one in which the start codon of the endogenous Rag1 gene has been deleted or has been disrupted or mutated such that the start codon is no longer functional. For example, the start codon can be disrupted by a deletion or insertion within the start codon. Alternatively the start codon can be mutated by, for example, by a substitution of one or more nucleotides. In another example, a 3′ fragment of the Rag1 gene can be deleted or disrupted (e.g., including the stop codon). In another example, an internal fragment of the Rag1 gene (i.e., a fragment from the middle of the Rag1 gene) can be deleted or disrupted. In another example, all of the coding sequence in the endogenous Rag1 gene is deleted or disrupted.

V(D)J recombination-activating protein 2 (also known as RAG2, recombination-activating 2, recombination activating gene 2, and recombination activating protein 2) is encoded by the Rag2 gene (also known as recombination activating 2). As mentioned above, RAG1 is a catalytic component of the RAG complex, a multiprotein complex that mediates the DNA cleavage phase during V(D)J recombination. RAG2 is not a catalytic component but is required for all known catalytic activities. RAG1 and RAG2 are essential to the generation of mature B cells and T cells, two types of lymphocyte that are crucial components of the adaptive immune system.

Mouse Rag2 maps to 2 E2; 2 53.87 cM on chromosome 2 (NCBI RefSeq Gene ID 19374; Assembly GRCm39 (GCF_000001635.27); location NC_000068.8 (101455057..101462873). Reference to the mouse Rag2 gene includes the canonical, wild type form as well as all allelic forms and isoforms. The canonical, wild type mouse RAG2 protein has been assigned UniProt accession number P21784 and NCBI Accession No. NP_033046.1. Reference to mouse RAG2 proteins includes canonical, wild type forms as well as all allelic forms and isoforms. An mRNA (cDNA) encoding the canonical isoform is assigned NCBI Accession No. NM_009020.3. Reference to the mouse Rag2 mRNA (cDNA) and coding sequence includes the canonical, wild type forms as well as all allelic forms and isoforms.

Rat Rag2 maps to 3q31 on chromosome 3 (NCBI RefSeq Gene ID 295953; Assembly mRatBN7.2 (GCF_015227675.2); location NC_051338.1 (87902373..87910227). Reference to the rat Rag2 gene includes the canonical, wild type form as well as all allelic forms and isoforms. The canonical, wild type rat RAG2 protein has been assigned UniProt accession number G3V6K7 and NCBI Accession No. NP_001093998.1. Reference to rat RAG2 proteins includes canonical, wild type forms as well as all allelic forms and isoforms. An mRNA (cDNA) encoding the canonical isoform is assigned NCBI Accession No. NM_001100528.1. Reference to the rat Rag2 mRNA (cDNA) and coding sequence includes the canonical, wild type forms as well as all allelic forms and isoforms.

An inactivated endogenous Rag2 gene is a Rag2 gene that does not produce a RAG2 protein or does not produce a functional RAG2 protein. The non-human animal (or cell or genome) can comprise the inactivated Rag2 gene in its germline. The non-human animal (or cell or genome) can be homozygous for an inactivating mutation in the Rag2 gene. As one example, an inactivated endogenous Rag2 gene can comprise an insertion, a deletion, or one or more point mutations in the endogenous Rag2 gene resulting in loss of expression of functional RAG2 protein. Some inactivated endogenous Rag2 genes can comprise a deletion or disruption of all of the endogenous Rag2 gene or can comprise a deletion or disruption of a fragment of (i.e., a part of or portion of) the endogenous Rag2 gene. For example, some, most, or all of the coding sequence in the endogenous Rag2 gene can be deleted or disrupted. In one example, a 5′ fragment of the Rag2 gene can be deleted or disrupted (e.g., including the start codon). As one example, an inactivated endogenous Rag2 gene can be one in which the start codon of the endogenous Rag2 gene has been deleted or has been disrupted or mutated such that the start codon is no longer functional. For example, the start codon can be disrupted by a deletion or insertion within the start codon. Alternatively the start codon can be mutated by, for example, by a substitution of one or more nucleotides. In another example, a 3′ fragment of the Rag2 gene can be deleted or disrupted (e.g., including the stop codon). In another example, an internal fragment of the Rag2 gene (i.e., a fragment from the middle of the Rag2 gene) can be deleted or disrupted. In another example, all of the coding sequence in the endogenous Rag2 gene is deleted or disrupted.

Interleukin 2 receptor subunit gamma (also known as interleukin 2 receptor, gamma; interleukin 2 receptor, gamma (severe combined immunodeficiency), isoform CRA_a; cytokine receptor common subunit gamma precursor) is encoded by the Il2rg gene (also known as interleukin 2 receptor subunit gamma or IL2RG). IL2RG is a cytokine receptor subunit that is common to receptor complexes for several different interleukin receptors. IL2RG is located on the surface of immature blood-forming cells in bone marrow. IL2RG partners with other proteins to direct blood-forming cells to form lymphocytes. IL2RG also directs the growth and maturation of T cells, B cells, and natural killer cells. Mutations in Il2rg can cause X-linked severe combined immunodeficiency in which lymphocytes cannot develop normally. A lack of functional mature lymphocytes disrupts the immune system's ability to protect the body from infection.

Mouse Il2rg maps to X D; X 43.9 cM on chromosome X (NCBI RefSeq Gene ID 16186; Assembly GRCm39 (GCF_000001635.27); location NC_000086.8 (100307991..100311861, complement). Reference to the mouse Il2rg gene includes the canonical, wild type form as well as all allelic forms and isoforms. The canonical, wild type mouse IL2rG protein has been assigned UniProt accession number P34902 and NCBI Accession No. NP_038591.1. Reference to mouse IL2RG proteins includes canonical, wild type forms as well as all allelic forms and isoforms. An mRNA (cDNA) encoding the canonical isoform is assigned NCBI Accession No. NM_013563.4. Reference to the mouse Il2rg mRNA (cDNA) and coding sequence includes the canonical, wild type forms as well as all allelic forms and isoforms.

Rat Il2rg maps to Xq22 on chromosome X (NCBI RefSeq Gene ID 140924; Assembly mRatBN7.2 (GCF_015227675.2); location NC_051356.1 (66395330..66399026, complement). Reference to the rat Il2rg gene includes the canonical, wild type form as well as all allelic forms and isoforms. The canonical, wild type rat IL2rG protein has been assigned UniProt accession number Q68FU6 and NCBI Accession No. NP_543165.1. Reference to rat IL2RG proteins includes canonical, wild type forms as well as all allelic forms and isoforms. An mRNA (cDNA) encoding the canonical isoform is assigned NCBI Accession No. NM_080889.1. Reference to the rat Il2rg mRNA (cDNA) and coding sequence includes the canonical, wild type forms as well as all allelic forms and isoforms.

An inactivated endogenous Il2rg gene is an Il2rg gene that does not produce a IL2RG protein or does not produce a functional IL2RG protein. The non-human animal (or cell or genome) can comprise the inactivated Il2rg gene in its germline. The non-human animal (or cell or genome) can be homozygous for an inactivating mutation in the Il2rg gene. As one example, an inactivated endogenous Il2rg gene can comprise an insertion, a deletion, or one or more point mutations in the endogenous Il2rg gene resulting in loss of expression of functional IL2RG protein. Some inactivated endogenous Il2rg genes can comprise a deletion or disruption of all of the endogenous Il2rg gene or can comprise a deletion or disruption of a fragment of (i.e., a part of or portion of) the endogenous Il2rg gene. For example, some, most, or all of the coding sequence in the endogenous Il2rg gene can be deleted or disrupted. In one example, a 5′ fragment of the Il2rg gene can be deleted or disrupted (e.g., including the start codon). As one example, an inactivated endogenous Il2rg gene can be one in which the start codon of the endogenous Il2rg gene has been deleted or has been disrupted or mutated such that the start codon is no longer functional. For example, the start codon can be disrupted by a deletion or insertion within the start codon. Alternatively the start codon can be mutated by, for example, by a substitution of one or more nucleotides. In another example, a 3′ fragment of the Il2rg gene can be deleted or disrupted (e.g., including the stop codon). In another example, an internal fragment of the Il2rg gene (i.e., a fragment from the middle of the Il2rg gene) can be deleted or disrupted. In another example, all of the coding sequence in the endogenous Il2rg gene is deleted or disrupted.

### Humanized Sirpa Loci

In certain aspects, the genetically modified non-human animals provided herein further express a human or humanized SIRPA protein encoded by a nucleic acid operably linked to a Sirpa promoter.

Signal regulatory proteins (SIRPs) constitute a family of cell surface glycoproteins which are expressed on lymphocytes, myeloid cells (including macrophages, neutrophils, granulocytes, myeloid dendritic cells, and mast cells) and neurons (e.g., see Barclay and Brown, 2006, Nat Rev Immunol 6, 457-464). The reported SIRP genes include at least SIRPA, SIRP3, SIRPβ, SIRPγ, and SIRP8 and can be categorized by their respective ligands and types of signaling in which they are involved. SIRPA (also referred to as CD172A, SHPS1, P84, MYD-1, BIT and PTPNS1) is expressed on immune cells of the myeloid lineage and functions as an inhibitory receptor via an immunoreceptor tyrosine-based inhibitory motif (ITIM). SIRPA expression has also been observed on neurons. Reported ligands for SIRPA include, most notably, CD47, but also include surfactant proteins A and D. The role of SIRPA, in particular, has been investigated in respect of its inhibitory role in the phagocytosis of host cells by macrophages. For example, CD47 binding to SIRPA on macrophages, triggers inhibitory signals that negatively regulates phagocytosis. Alternatively, positive signaling effects mediated through SIRPA binding have been reported (Shultz et al., 1995, J Immunol 154, 180-91). SIRPA has been shown to improve cell engraftment in immunodeficient mice (Strowig et al. Proc Natl Acad Sci USA 2011; 108: 13218-13223).

Polypeptide sequences for wild-type human SIRPA and the nucleic acid sequences that encode wild-type human SIRPA may be found at Genbank Accession Nos. NP_001035111.1 and NM_001040022.1 (isoform 1 and transcript variant 1); NP_001035112.1 and NM_001040023.2 (isoform 1 and transcript variant 2); NP_001317657.1 and NM_001330728.1 (isoform 2 and transcript variant 4); and NP_542970.1 and NM_080792.3 (isoform 1 and transcript variant 3). The SIRPA gene is conserved in at least chimpanzee, Rhesus monkey, dog, cow, mouse, rat, and chicken. The genomic locus encoding the wild-type human SIRPA protein may be found in the human genome at Chromosome 20; NC_000020.11 (1894167-1940592). In some embodiments, human SIRPA protein is encoded by exons 2 through 9 at this locus. As such, in some embodiments, a nucleic acid sequence including coding sequence for human SIRPA includes one or more of exons 2-9 of the human SIRPA gene. In some instances, the nucleic acid sequence also includes aspects of the genomic locus of the human SIRPA, e.g., introns, 3′ and/or 5′ untranslated sequence (UTRs). In some instances, the nucleic acid sequence includes whole regions of the human SIRPA genomic locus. In some instances, the nucleic acid sequence includes exons 2-4 of the human SIRPA genomic locus.

Exemplary humanized Sirpa sequences are set forth in Table 3. For protein sequences, signal peptides are underlined, and transmembrane and cytoplasmic sequences are italicized. Representative mouse Sirpa cDNA, mouse Sirpa protein, human SIRPA cDNA, and human SIRPA protein sequences are described in U.S. Pat. No. 11,019,810, which is incorporated by reference herein in its entirety.

TABLE 3
Humanized Sirpa Protein (SEQ ID NO: 12)

MEPAGPAPGRLGPLLLCLLLSASCFCTGVAGEEELQVIQPDKSVLV
AAGETATLRCTATSLIPVGPIQWERGAGPGRELIYNQKEGHFPRV
TTVSDLTKRNNMDFSIRIGNITPADAGTYYCVKFRKGSPDDVEFK
SGAGTELSVRAKPSAPVVSGPAARATPQHTVSFTCESHGFSPRDI
TLKWFKNGNELSDFQTNVDPVGESVSYSIHSTAKVVLTREDVHSQ
VICEVAHVTLQGDPLRGTANLSETIRVPPTLEVTQQPVRAENQVN
VTCQVRKFYPQRLQLTWLENGNVSRTETASTVTENKDGTYNWMSW
LLVNVSAHRDDVKLTCQVEHDGQPAVSKSHDLKVSAHPKEQGSNT
AADNNATHNWNVFIGVGVACALLVVLLMAALYLLRIKQKKAKGST
SSTRLHEPEKNAREITQIQDINDINDITYADLNLPKEKKPAPRAP
EPNNHTEYASIETGKVPRPEDTLTYADLDMVHLSRAQPAPKPEPS
FSEYASVQVQRK

In some embodiments, the non-human animals provided herein express humanized Sirpa proteins on the surface of immune cells (e.g., myeloid cells) of the non-human animals resulting from a genetic modification of an endogenous locus of the non-human animal that encodes a Sirpa protein. Suitable examples described herein include rodents, for example, mice.

A humanized Sirpa gene, in some embodiments, comprises genetic material from a heterologous species (e.g., humans), wherein the humanized Sirpa gene encodes a Sirpa protein that comprises the encoded portion of the genetic material from the heterologous species. In some embodiments, a humanized Sirpa gene of the present disclosure comprises genomic DNA of a heterologous species that corresponds to the extracellular portion of a SIRPA protein that is expressed on the plasma membrane of a cell. Non-human animals, embryos, cells and targeting constructs for making non-human animals, non-human embryos, and cells containing said humanized Sirpa gene are also provided.

In some embodiments, an endogenous non-human animal (e.g., rodent) Sirpa gene is deleted. In some embodiments, an endogenous non-human animal (e.g., rodent) Sirpa gene is altered, wherein a portion of the endogenous non-human animal (e.g., rodent) Sirpa gene is replaced with a heterologous sequence (e.g., a human SIRPA sequence in whole or in part). In some embodiments, all or substantially all of an endogenous non-human animal (e.g., rodent) Sirpa gene is replaced with a heterologous gene (e.g., a human SIRPA gene). In some embodiments, a portion of a heterologous SIRPα gene is inserted into an endogenous non-human Sirpa gene at an endogenous non-human animal (e.g., rodent) Sirpa locus. In some embodiments, the heterologous gene is a human gene. In some embodiments, the modification or humanization is made to one of the two copies of the endogenous non-human animal (e.g., rodent) Sirpa gene, giving rise to a non-human animal which is heterozygous with respect to the humanized Sirpa gene. In other embodiments, a non-human animal is provided that is homozygous for a humanized Sirpa gene. In some embodiments, all of an endogenous non-human animal (e.g., rodent) Sirpa gene is replaced with a portion of heterologous gene (e.g., a portion of human SIRPA gene), such that the genetically modified non-human animal (e.g., rodent) expresses a functional fragment of a full-length human SIRPA polypeptide (e.g., an extracellular domain of a human SIRPA polypeptide).

A non-human animal of the present disclosure contains a human SIRPA gene in whole or in part at an endogenous non-human Sirpa locus. Thus, such non-human animals can be described as having a heterologous SIRP gene. The replaced, inserted or modified SIRPα gene at the endogenous non-human animal (e.g., rodent) Sirpa locus can be detected using a variety of methods including, for example, PCR, Western blot, Southern blot, restriction fragment length polymorphism (RFLP), or a gain or loss of allele assay. In some embodiments, the non-human animal is heterozygous with respect to the humanized Sirpa gene.

In various embodiments, a humanized Sirpa gene according to the present disclosure includes a SIRPα gene that has a second, third and fourth exon each having a sequence at least 50% (e.g., 50%, 55%, 60%, 65%, 70%, 75%, 80%, 85%, 90%, 91%, 92%, 93%, 94%, 95%, 96%, 97%, 98%, 99% or more) identical to a second, third and fourth exon that appear in a human SIRPA gene.

In various embodiments, a humanized Sirpa gene according to the present disclosure includes a SIRPα gene that has a nucleotide coding sequence (e.g., a cDNA sequence) at least 50% (e.g., 50%, 55%, 60%0, 65%, 70%, 75%, 80%, 85%, 90%, 91%, 92%, 93%, 94%, 95%, 96%, 97%, 98%, 99% or more) identical to nucleotides 352-1114 that appear in a human SIRPA cDNA sequence.

In various embodiments, a humanized Sirpa protein produced by a non-human animal of the present disclosure has an extracellular portion having a sequence that is at least 50% (e.g., 50%, 55%, 60%, 65%, 70%, 75%, 80%, 85%, 90%, 91%, 92%, 93%, 94%, 95%, 96%, 97%, 98%, 99% or more) identical to an extracellular portion of a human SIRPA protein.

In various embodiments, a humanized Sirpa a protein produced by a non-human animal of the present disclosure has an extracellular portion having a sequence that is at least 50% (e.g., 50%, 55%, 60%, 65%, 70%, 75%, 80%, 85%, 90%, 91%, 92%, 93%, 94%, 95%, 96%, 97%, 98%, 99% or more) identical to amino acid residues 28-362 that appear in a human SIRPA protein.

In various embodiments, a humanized Sirpa protein produced by a non-human animal of the present disclosure has an amino acid sequence that is at least 50% (e.g., 50%, 55%, 60%, 65%, 70%, 75%, 80%, 85%, 90%, 91%, 92%, 93%, 94%, 95%, 96%, 97%, 98%, 99% or more) identical to an amino acid sequence of a humanized Sirpa protein that appears in Table 3.

Compositions and methods for making non-human animals that express a humanized Sirpa protein, including specific polymorphic forms or allelic variants (e.g., single amino acid differences), are provided, including compositions and methods for making non-human animals that express such proteins from a human promoter and a human regulatory sequence. In some embodiments, compositions and methods for making non-human animals that express such proteins from an endogenous promoter and an endogenous regulatory sequence are also provided. The methods include inserting the genetic material encoding a human SIRPA protein in whole or in part at a precise location in the genome of a non-human animal that corresponds to an endogenous non-human animal (e.g., rodent) Sirpa gene thereby creating a humanized Sirpa gene that expresses a SIRPA protein that is human in whole or in part. In some embodiments, the methods include inserting genomic DNA corresponding to exons 2-4 of a human SIRPA gene into an endogenous non-human animal (e.g., rodent) Sirpa gene of the non-human animal thereby creating a humanized gene that encodes a Sirpa protein that contains a human portion containing amino acids encoded by the inserted exons.

In various embodiments, a humanized Sirpa gene approach employs a relatively minimal modification of the endogenous gene and results in natural Sirpa-mediated signal transduction in the non-human animal. Thus, in such embodiments, the Sirpa gene modification does not affect other surrounding genes or other endogenous non-human animal (e.g., rodent) Sirp genes. Further, in various embodiments, the modification does not affect the assembly of a functional receptor on the plasma and maintains normal effector functions via binding and subsequent signal transduction through the cytoplasmic portion of the receptor which is unaffected by the modification.

In addition to mice having humanized Sirpa genes as described herein, also provided herein are other genetically modified non-human animals that comprise humanized Sirpa genes. In some embodiments, such non-human animals comprise a humanized Sirpa gene operably linked to an endogenous Sirpa promoter. In some embodiments, such non-human animals express a humanized Sirpa protein from an endogenous locus, wherein the humanized Sirpa protein comprises amino acid residues 28-362 of a human SIRPA protein.

Humanized Sirpa polypeptides, loci encoding humanized Sirpa polypeptides and non-human animals expressing humanized Sirpa polypeptides are described in U.S. Pat. No. 11,019,810, WO 2014/039782, WO 2014/071397, and WO 2016/168212, U.S. Pat. No. 10,918,095, each of which is incorporated by reference herein in its entirety.

### Humanized GM-CSF Loci

In some aspects, the genetically modified non-human animals provided herein further express a human GM-CSF protein encoded by a nucleic acid operably linked to a GM-CSF promoter. By a human GM-CSF protein, it is a meant a protein that is human GM-CSF or is substantially identical to human GM-CSF, e.g., it is 80% or more identical, 85% or more identical, 90% or more identical, or 95% or more identical to human GM-CSF, for example, 97%, 98%, or 99% identical to human GM-CSF. A nucleic acid sequence that encodes a human GM-CSF protein is, therefore, a polynucleotide that comprises coding sequence for a human GM-CSF protein, i.e., human GM-CSF or a protein that is substantially identical to human GM-CSF.

GM-CSF is a cytokine crucial for myeloid cell development and function. GM-CSF is not cross-reactive between human and mouse. GM-CSF is highly expressed in the lung and important for lung homeostasis in vivo, as demonstrated by the fact that GM-CSF KO mice develop pulmonary alveolar proteinosis (PAP) which is characterized by protein accumulation in the lung due to defective surfactant clearance. Alveolar macrophages from GM-CSF KO mice have a defect in terminal differentiation, which leads to impaired innate immunity to pathogens in the lung. GM-CSF also stimulates the proliferation of human alveolar macrophages (AM) in vitro. GM-CSF is largely dispensable for steady-state hematopoiesis. In contrast, GM-CSF is required for inflammatory responses such as the production of proinflammatory cytokines by macrophages and the mobilization and recruitment of monocytes. GM-CSF is also essential for protective immunity against a range of pathogens, including M. tuberculosis. In particular, GM-CSF KO mice infected with M. tuberculosis do not develop granulomas, a hallmark of tuberculosis.

Polypeptide sequence for human GM-CSF and the nucleic acid sequence that encodes for human GM-CSF may be found at Genbank Accession Nos. NP_000749.2 and NM_000758.4, respectively. The genomic locus encoding the human GM-CSF protein may be found in the human genome at Chromosome 5; NG_033024.1 (4998-7379). Protein sequence is encoded by exons 1 through 4 at this locus. As such, a nucleic acid sequence comprising coding sequence for human GM-CSF comprises one or more of exons 1-4 of the human GM-CSF gene. In some instances, the nucleic acid sequence also comprises aspects of the genomic locus of the human GM-CSF, e.g., introns, 3′ and/or 5′ untranslated sequence (UTRs). In some instances, the nucleic acid sequence comprises whole regions of the human GM-CSF genomic locus.

In some embodiments, in the genetically modified non-human animals provided herein, the nucleic acid sequence that encodes a human GM-CSF protein is operably linked to one or more regulatory sequences of the non-human animal (e.g., mouse) GM-CSF gene. Non-human animal (e.g., mouse) GM-CSF regulatory sequences are those sequences of the non-human animal (e.g., mouse) GM-CSF genomic locus that regulate non-human animal (e.g., mouse) GM-CSF expression, for example, 5′ regulatory sequences, e.g., the GM-CSF promoter, GM-CSF 5′ untranslated region (UTR), etc.; 3′ regulatory sequences, e.g., the 3′UTR; and enhancers, etc. For example, mouse GM-CSF is located on chromosome 11, GRCm39, NC_000077.7, at about positions c54140725-54138096, and the mouse GM-CSF coding sequence may be found at Genbank Accession No. NM_009969.4. The regulatory sequences of mouse GM-CSF are well defined in the art, and may be readily identified using in silico methods, e.g., by referring to the above Genbank Accession Nos. on the UCSC Genome Browser, on the world wide web at genome.ucsc.edu, or by experimental methods as described in the art. In some instances, e.g., when the nucleic acid sequence that encodes a human GM-CSF protein is located at the non-human animal (e.g., mouse) GM-CSF genomic locus, the regulatory sequences operably linked to the human GM-CSF coding sequence are endogenous, or native, to the non-human animal (e.g., mouse) genome, i.e., they were present in the non-human animal (e.g., mouse) genome prior to integration of human nucleic acid sequences.

In some instances, the genetically modified non-human animal expressing a human GM-CSF protein is generated by the random integration, or insertion, of human nucleic acid sequence encoding human GM-CSF protein or a fragment thereof, i.e., “human GM-CSF nucleic acid sequence”, or “human GM-CSF sequence”, into the genome of the non-human animal. Typically, in such embodiments, the location of the nucleic acid sequence encoding a human GM-CSF protein in the genome is unknown. In other instances, the genetically modified non-human animal expressing a human GM-CSF protein is generated by the targeted integration, or insertion, of human GM-CSF nucleic acid sequence into the genome of the non-human animal, by, for example, homologous recombination. In homologous recombination, a polynucleotide is inserted into the host genome at a target locus while simultaneously removing host genomic material, e.g., 50 base pairs (bp) or more, 100 bp or more, 200 bp or more, 500 bp or more, 1 kB or more, 2 kB or more, 5 kB or more, 10 kB or more, 15 kB or more, 20 kB or more, or 50 kB or more of genomic material, from the target locus. So, for example, in a genetically modified non-human animal (e.g., mouse) comprising a nucleic acid sequence that encodes a human GM-CSF protein created by targeting human GM-CSF nucleic acid sequence to the non-human animal GM-CSF (e.g., mouse) locus, human GM-CSF nucleic acid sequence may replace some or all of the non-human animal (e.g., mouse) sequence, e.g., exons and/or introns, at the GM-CSF locus. In some such instances, human GM-CSF nucleic acid sequence is integrated into the non-human animal (e.g., mouse) GM-CSF locus such that expression of the human GM-CSF sequence is regulated by the native, or endogenous, regulatory sequences at the non-human animal (e.g., mouse) GM-CSF locus. In other words, the regulatory sequence(s) to which the nucleic acid sequence encoding a human GM-CSF protein is operably linked are the native GM-CSF regulatory sequences at the non-human animal (e.g., mouse) GM-CSF locus.

In some instances, the integration of human GM-CSF sequence does not affect the transcription of the gene into which the human GM-CSF sequence has integrated. For example, if the human GM-CSF sequence integrates into coding sequence as an intein, or the human GM-CSF sequence comprises a 2A peptide, the human GM-CSF sequence will be transcribed and translated simultaneously with the gene into which the human GM-CSF sequence has integrated. In other instances, the integration of the human GM-CSF sequence interrupts the transcription of the gene into which the human GM-CSF sequence has integrated. For example, upon integration of the human GM-CSF sequence by homologous recombination, some or all of the coding sequence at the integration locus may be removed, such that the human GM-CSF sequence is transcribed instead. In some such instances, the integration of human GM-CSF sequence creates a null mutation, and hence, a null allele. A null allele is a mutant copy of a gene that completely lacks that gene's normal function. This can be the result of the complete absence of the gene product (protein, RNA) at the molecular level, or the expression of a non-functional gene product. At the phenotypic level, a null allele includes a deletion of the entire locus.

In some instances, the genetically modified non-human animal (e.g., mouse) expressing a human GM-CSF protein comprises one copy of the nucleic acid sequence encoding a human GM-CSF protein. For example, the non-human animal (e.g., mouse) may be heterozygous for the nucleic acid sequence. In other words, one allele at a locus will comprise the nucleic acid sequence, while the other will be the endogenous allele. For example, as discussed above, in some instances, human GM-CSF nucleic acid sequence is integrated into the non-human animal (e.g., mouse) GM-CSF locus such that it creates a null allele for non-human animal (e.g., mouse) GM-CSF. In some such embodiments, the humanized GM-CSF mouse may be heterozygous for the nucleic acid sequence encoding, i.e., the humanized GM-CSF mouse comprises one null allele for non-human animal (e.g., mouse) GM-CSF (the allele comprising the nucleic acid sequence) and one endogenous GM-CSF allele (wild type or otherwise). In other instances, the genetically modified non-human animal (e.g., mouse) expressing a human GM-CSF protein comprises two copies of the nucleic acid sequence encoding a human GM-CSF protein. For example, the non-human animal (e.g., mouse) may be homozygous for the nucleic acid sequence, i.e., both alleles for a locus in the diploid genome will comprise the nucleic acid sequence, i.e., the genetically modified non-human animal (e.g., mouse) expressing a human GM-CSF protein comprises two null alleles for the mouse GM-CSF (the allele comprising the nucleic acid sequence).

Although embodiments employing a human GM-CSF gene in a mouse are extensively discussed herein, other non-human animals (e.g., rodents, e.g., rats) that comprise a human GM-CSF gene are also provided.

Human GM-CSF polypeptides, loci encoding human GM-CSF polypeptides and non-human animals expressing human GM-CSF polypeptides are described in WO2011/044050, WO 2014/039782 and WO 2014/071397, each of which is incorporated by reference herein.

### Humanized IL-3 Loci

In some aspects, the genetically modified non-human animals provided herein further express a human IL-3 protein encoded by a nucleic acid operably linked to an IL-3 promoter. By a human IL-3 protein, it is a meant a protein that is human IL-3 or is substantially identical to human IL-3, e.g., it is 80% or more identical, 85% or more identical, 90% or more identical, or 95% or more identical to human IL-3, for example, 97%, 98%, or 99% identical to human IL-3. A nucleic acid sequence that encodes a human IL-3protein is, therefore, a polynucleotide that comprises coding sequence for a human IL-3protein, i.e., human IL-3 or a protein that is substantially identical to human IL-3.

Like GM-CSF, IL-3 is a cytokine crucial for myeloid cell development and function. IL-3 is not cross-reactive between human and mouse. IL-3 stimulates early hematopoietic progenitors in vitro, but is dispensable for steady-state hematopoiesis in vivo. However, together with GM-CSF it is required for effective DTH responses in vivo. IL-3 also specifically stimulates the proliferation of alveolar macrophages (AM) in vitro.

Polypeptide sequence for human IL-3 and the nucleic acid sequence that encodes for human IL-3 may be found at Genbank Accession Nos. NP_000579.2 and NM_000588.4. The genomic locus encoding the human IL-3 protein may be found in the human genome at chromosome 5, GRCh38.p14; NC_000005.10 (132060655-132063204). Protein sequence is encoded by exons 1 through 5 at this locus. As such, a nucleic acid sequence comprising coding sequence for human IL-3 comprises one or more of exons 1-5 of the human IL-3 gene. In some instances, the nucleic acid sequence also comprises aspects of the genomic locus of the human IL-3, e.g., introns, 3′ and/or 5′ untranslated sequence (UTRs). In some instances, the nucleic acid sequence comprises whole regions of the human IL-3 genomic locus.

In some embodiments, in the genetically modified non-human animals provided herein, the nucleic acid sequence that encodes a human IL-3 protein is operably linked to one or more regulatory sequences of the non-human animal (e.g., mouse) IL-3 gene. Non-human animal (e.g., mouse) IL-3 regulatory sequences are those sequences of the non-human animal (e.g., mouse) IL-3 genomic locus that regulate non-human animal (e.g., mouse) IL-3 expression, for example, 5′ regulatory sequences, e.g., the IL-3 promoter, IL-3 5′ untranslated region (UTR), etc.; 3′ regulatory sequences, e.g., the 3′UTR; and enhancers, etc. For example, mouse IL-3 is located on chromosome 11, GRCm39, NC_000077.7, at about positions c54158105-54155911, and the mouse IL-3 coding sequence may be found at Genbank Accession No. NM_010556.4. The regulatory sequences of mouse IL-3 are well defined in the art, and may be readily identified using in silico methods, e.g., by referring to the above Genbank Accession Nos. on the UCSC Genome Browser, on the world wide web at genome.ucsc.edu, or by experimental methods as described in the art. In some instances, e.g., when the nucleic acid sequence that encodes a human IL-3 protein is located at the non-human animal (e.g., mouse) IL-3 genomic locus, the regulatory sequences operably linked to the human IL-3 coding sequence are endogenous, or native, to the non-human animal (e.g., mouse) genome, i.e., they were present in the non-human animal (e.g., mouse) genome prior to integration of human nucleic acid sequences.

In some instances, the genetically modified non-human animal expressing a human IL-3 protein is generated by the random integration, or insertion, of human nucleic acid sequence encoding human IL-3 protein or a fragment thereof, i.e., “human IL-3 nucleic acid sequence”, or “human IL-3 sequence”, into the genome of the non-human animal. Typically, in such embodiments, the location of the nucleic acid sequence encoding a human IL-3 protein in the genome is unknown. In other instances, the genetically modified non-human animal expressing a human IL-3 protein is generated by the targeted integration, or insertion, of human IL-3 nucleic acid sequence into the genome of the non-human animal, by, for example, homologous recombination. In homologous recombination, a polynucleotide is inserted into the host genome at a target locus while simultaneously removing host genomic material, e.g., 50 base pairs (bp) or more, 100 bp or more, 200 bp or more, 500 bp or more, 1 kB or more, 2 kB or more, 5 kB or more, 10 kB or more, 15 kB or more, 20 kB or more, or 50 kB or more of genomic material, from the target locus. So, for example, in a genetically modified non-human animal (e.g., mouse) comprising a nucleic acid sequence that encodes a human IL-3 protein created by targeting human IL-3 nucleic acid sequence to the non-human animal IL-3 (e.g., mouse) locus, human IL-3 nucleic acid sequence may replace some or all of the non-human animal (e.g., mouse) sequence, e.g., exons and/or introns, at the IL-3 locus. In some such instances, human IL-3 nucleic acid sequence is integrated into the non-human animal (e.g., mouse) IL-3 locus such that expression of the human IL-3 sequence is regulated by the native, or endogenous, regulatory sequences at the non-human animal (e.g., mouse) IL-3 locus. In other words, the regulatory sequence(s) to which the nucleic acid sequence encoding a human IL-3 protein is operably linked are the native IL-3 regulatory sequences at the non-human animal (e.g., mouse) IL-3 locus.

In some instances, the integration of human IL-3 sequence does not affect the transcription of the gene into which the human IL-3 sequence has integrated. For example, if the human IL-3 sequence integrates into coding sequence as an intein, or the human IL-3 sequence comprises a 2A peptide, the human IL-3 sequence will be transcribed and translated simultaneously with the gene into which the human IL-3 sequence has integrated. In other instances, the integration of the human IL-3 sequence interrupts the transcription of the gene into which the human IL-3 sequence has integrated. For example, upon integration of the human IL-3 sequence by homologous recombination, some or all of the coding sequence at the integration locus may be removed, such that the human IL-3 sequence is transcribed instead. In some such instances, the integration of human IL-3 sequence creates a null mutation, and hence, a null allele. A null allele is a mutant copy of a gene that completely lacks that gene's normal function. This can be the result of the complete absence of the gene product (protein, RNA) at the molecular level, or the expression of a non-functional gene product. At the phenotypic level, a null allele includes a deletion of the entire locus.

In some instances, the genetically modified non-human animal (e.g., mouse) expressing a human IL-3 protein comprises one copy of the nucleic acid sequence encoding a human IL-3 protein. For example, the non-human animal (e.g., mouse) may be heterozygous for the nucleic acid sequence. In other words, one allele at a locus will comprise the nucleic acid sequence, while the other will be the endogenous allele. For example, as discussed above, in some instances, human IL-3 nucleic acid sequence is integrated into the non-human animal (e.g., mouse) IL-3 locus such that it creates a null allele for non-human animal (e.g., mouse) IL-3. In some such embodiments, the humanized IL-3 mouse may be heterozygous for the nucleic acid sequence encoding, i.e., the humanized IL-3 mouse comprises one null allele for non-human animal (e.g., mouse) IL-3 (the allele comprising the nucleic acid sequence) and one endogenous IL-3 allele (wild type or otherwise). In other instances, the genetically modified non-human animal (e.g., mouse) expressing a human IL-3 protein comprises two copies of the nucleic acid sequence encoding a human IL-3 protein. For example, the non-human animal (e.g., mouse) may be homozygous for the nucleic acid sequence, i.e., both alleles for a locus in the diploid genome will comprise the nucleic acid sequence, i.e., the genetically modified non-human animal (e.g., mouse) expressing a human IL-3 protein comprises two null alleles for the mouse IL-3 (the allele comprising the nucleic acid sequence).

Although embodiments employing a human IL-3 gene in a mouse are extensively discussed herein, other non-human animals (e.g., rodents, e.g., rats) that comprise a human IL-3 gene are also provided.

Human IL-3 polypeptides, loci encoding human IL-3 polypeptides and non-human animals expressing human IL-3 polypeptides are described in WO2011/044050, WO 2014/039782 and WO 2014/071397, each of which is incorporated by reference herein.

### Genetically Modified Non-Human Animals and ES Cells

In certain aspects, provided herein are genetically modified non-human animals (e.g., rodents, such as rats or mice) comprising: (i) a homozygous null mutation in Rag1 and/or Rag2 gene (e.g., a Rag 1 and/or Rag2 gene knock-out); (ii) a homozygous null mutation in IL2rg gene (e.g., a IL2rg gene knock-out); (iii) a homozygous null mutation in the non-human animal FMS-like tyrosine kinase 3 (Flt3) gene, and (iv) a Flt3 ligand (Flt31) gene that comprises a non-human animal portion and a human portion operably linked to a Flt31 promoter, and optionally one or more of the humanized loci disclosed herein as well as genetically modified non-human animal ES cells useful in the making of such non-human animals.

In certain aspects, provided herein are genetically modified non-human animals (e.g., rodents, such as rats or mice) and non-human animal (e.g., rodents, such as rats or mice) ES cells comprising in their germline and/or genome: (i) a homozygous null mutation in Rag1 and/or Rag2 gene (e.g., a Rag 1 and/or Rag2 gene knock-out); (ii) a homozygous null mutation in IL2rg gene (e.g., a IL2rg gene knock-out); (iii) a homozygous null mutation in the non-human animal FMS-like tyrosine kinase 3 (Flt3) gene, and (iv) a Flt3 ligand (Flt31) gene that comprises a non-human animal portion and a human portion operably linked to a Flt31 promoter, and optionally one or more of the engineered loci described herein. In some embodiments, provided herein are genetically modified non-human animals (e.g., mice) and non-human animal (e.g., mouse) ES cells comprising in their germline and/or genome: (i) a Rag2 gene knock-out; (ii) a IL2rg gene knock-out; (iii) a homozygous null mutation in the non-human animal FMS-like tyrosine kinase 3 (Flt3) gene, and (iv) a Flt3 ligand (Flt31) gene that comprises a non-human animal portion and a human portion operably linked to a Flt31 promoter, and optionally one or more of the engineered loci described herein. For example, in some embodiments the non-human animal or ES cell comprises in its germline and/or genome a humanized Sirpa locus provided herein. In certain embodiments, the non-human animal or ES cell comprises in their germline and/or genome a GM-CSF locus provided herein. In certain embodiments, the non-human animal or ES cell comprises in their germline and/or genome an IL-3 locus provided herein. In some embodiments, the non-human animal or ES cell is heterozygous for one or more of the loci, e.g., genetically engineered loci, provided herein. In some embodiments, the non-human animal or ES cell is homozygous for one or more of the loci, e.g., genetically engineered loci, provided herein.

In some embodiments, the non-human animal can be any non-human animal. In some embodiments, the non-human animal is a vertebrate. In some embodiments, the non-human animal is a mammal. In some embodiments, the genetically modified non-human animal described herein may be selected from a group consisting of a mouse, rat, rabbit, pig, bovine (e.g., cow, bull, buffalo), deer, sheep, goat, llama, chicken, cat, dog, ferret, primate (e.g., marmoset, rhesus monkey). For non-human animals where suitable genetically modifiable ES cells are not readily available, other methods can be employed to make a non-human animal comprising the genetic modifications described herein. Such methods include, for example, modifying a non-ES cell genome (e.g., a fibroblast or an induced pluripotent cell) and employing nuclear transfer to transfer the modified genome to a suitable cell, such as an oocyte, and gestating the modified cell (e.g., the modified oocyte) in a non-human animal under suitable conditions to form an embryo.

In some embodiments, the non-human animal is a mammal. In some embodiments, the non-human animal is a small mammal, e.g., of the superfamily Dipodoidea or Muroidea. In some embodiments, the non-human animal is a rodent. In certain embodiments, the rodent is a mouse, a rat or a hamster. In some embodiments, the rodent is selected from the superfamily Muroidea. In some embodiments, the non-human animal is from a family selected from Calomyscidae (e.g., mouse-like hamsters), Cricetidae (e.g., hamster, New World rats and mice, voles), Muridae (e.g., true mice and rats, gerbils, spiny mice, crested rats), Nesomyidae (e.g., climbing mice, rock mice, white-tailed rats, Malagasy rats and mice), Platacanthomyidae (e.g., spiny dormice), and Spalacidae (e.g., mole rates, bamboo rats, and zokors). In some embodiments, the rodent is selected from a true mouse or rat (family Muridae), a gerbil, a spiny mouse, and a crested rat. In some embodiments, the mouse is from a member of the family Muridae. In some embodiments, the non-human animal is a rodent. In some embodiments, the rodent is selected from a mouse and a rat. In some embodiments, the non-human animal is a mouse.

In some embodiments, the non-human animal is a mouse of a C57BL strain. In some embodiments, the C57BL strain is selected from C57BL/A, C57BL/An, C57BL/GrFa, C57BL/KaLwN, C57BL/6, C57BL/6J, C57BL/6ByJ, C57BL/6NJ, C57BL/10, C57BL/10ScSn, C57BL/10Cr, and C57BL/Ola. In some embodiments, the non-human animal is a mouse of a 129 strain. In some embodiments, the 129 strain is selected from the group consisting of a strain that is 129P1, 129P2, 129P3, 129X1, 129S1 (e.g., 12951/SV, 12951/SvIm), 12952, 12954, 12955, 12959/SvEvH, 12956 (129/SvEvTac), 12957, 12958, 129T1, 129T2. In some embodiments, the genetically modified mouse is a mix of a 129 strain and a C57BL strain. In some embodiments, the mouse is a mix of 129 strains and/or a mix of C57BL/6 strains. In some embodiments, the 129 strain of the mix is a 12956 (129/SvEvTac) strain. In some embodiments, the mouse is a BALB strain (e.g., BALB/c). In some embodiments, the mouse is a mix of a BALB strain and another strain (e.g., a C57BL strain and/or a 129 strain). In some embodiments, the non-human animals provided herein can be a mouse derived from any combination of the aforementioned strains.

In some embodiments, the non-human animal provided herein is a rat. In some embodiments, the rat is selected from a Wistar rat, an LEA strain, a Sprague Dawley strain, a Fischer strain, F344, F6, and Dark Agouti. In some embodiments, the rat strain is a mix of two or more strains selected from the group consisting of Wistar, LEA, Sprague Dawley, Fischer, F344, F6, and Dark Agouti.

In certain embodiments, the genetically modified non-human animals or ES cells comprise in their genome and/or germline multiple loci provided herein, such as multiple genetically engineered loci provided herein. For example, in some embodiments the non-human animal or ES cell comprises in its germline and/or genome: (i) a homozygous null mutation in Rag1 and/or Rag2 gene (e.g., a Rag1 and/or Rag2 gene knock-out); (ii) a homozygous null mutation in IL2rg gene (e.g., a IL2rg gene knock-out); (iii) a homozygous null mutation in the non-human animal FMS-like tyrosine kinase 3 (Flt3) gene, and (iv) a Flt3 ligand (Flt31) gene that comprises a non-human animal portion and a human portion operably linked to a Flt31 promoter, provided herein. In some embodiments the non-human animal or ES cell comprises in its germline and/or genome (i) a homozygous null mutation in Rag1 and/or Rag2 gene (e.g., a Rag1 and/or Rag2 gene knock-out); (ii) a homozygous null mutation in IL2rg gene (e.g., a IL2rg gene knock-out); (iii) a homozygous null mutation in the non-human animal FMS-like tyrosine kinase 3 (Flt3) gene, and (iv) a Flt3 ligand (Flt31) gene that comprises a non-human animal portion and a human portion operably linked to a Flt31 promoter, and (v) a humanized Sirpa locus provided herein. In some embodiments, the non-human animal or ES cell comprises in its germline and/or genome (i) a homozygous null mutation in Rag1 and/or Rag2 gene (e.g., a Rag1 and/or Rag2 gene knock-out); (ii) a homozygous null mutation in IL2rg gene (e.g., a IL2rg gene knock-out); (iii) a homozygous null mutation in the non-human animal FMS-like tyrosine kinase 3 (Flt3) gene, (iv) a Flt3 ligand (Flt31) gene that comprises a non-human animal portion and a human portion operably linked to a Flt31 promoter, (v) a humanized Sirpa locus provided herein, and (vi) a humanized GM-CSF locus provided herein and/or a humanized IL-3 locus provided herein.

In certain aspects, the genetically modified non-human animal does not express a Flt3 polypeptide. In certain aspects, the genetically modified non-human animal express both soluble and membrane-bound forms of the Flt31 polypeptide, optionally the soluble and membrane-bound forms of the Flt31 polypeptide comprise a signal peptide and a cytokine-like core domain of a human FLT3L polypeptide.

In certain aspects, the genetically modified non-human animal expresses one or more of the human or humanized polypeptides encoded by the humanized loci provided herein. For example, in some embodiments the non-human animal expresses a human or humanized Sirpa polypeptide. In certain embodiments, the non-human animal expresses a human or humanized GM-CSF polypeptide. In certain embodiments, the non-human animal expresses a human or humanized IL-3 polypeptide.

The genetically modified non-human animals and ES cells can be generated using any appropriate method known in the art. For example, such genetically modified non-human animal ES cells can be generated using VELOCIGENE® technology, which is described in U.S. Pat. Nos. 6,586,251, 6,596,541, 7,105,348, and Valenzuela et al. (2003) “High-throughput engineering of the mouse genome coupled with high-resolution expression analysis” Nat. Biotech. 21(6): 652-659, each of which is hereby incorporated by reference. Modifications can also be made using a genome targeted nuclease system, such as a CRISPR/Cas system, a transcription activator-like effector nuclease (TALEN) system or a zinc finger nuclease (ZFN) system. In some embodiments, modifications are made using a CRISPR/Cas system, as described, for example, in U.S. patent application Ser. Nos. 14/314,866, 14/515,503, 14/747,461 and 14/731,914, each of which is incorporated by reference. Genetically modified rat ES cells and rats can be made according to US 2014/0235933 A1 (Regeneron Pharmaceuticals, Inc.), US 2014/0310828 A1 (Regeneron Pharmaceuticals, Inc.), Tong et al. (2010) Nature 467:211-215, and Tong et al. (2011) Nat Protoc. 6(6): doi:10.1038/nprot.2011.338 (all of which are incorporated herein by reference in their entireties). Exemplary methods of making such genetically modified non-human animals and ES cells are also provided herein in Example 1.

ES cells described herein can then be used to generate a non-human animal using methods known in the art. For example, the mouse non-human animal ES cells described herein can be used to generate genetically modified mice using the VELOCIMOUSE® method, as described in U.S. Pat. No. 7,294,754 and Poueymirou et al., Nature Biotech 25:91-99 (2007), each of which is hereby incorporated by reference. Resulting mice can be bread to homozygosity.

### Methods of Making Genetically Modified Non-Human Animals and ES Cells

In certain aspects, provided herein are methods of making non-human animals (e.g., a mouse or a rat) and ES cells that comprise one or more of the genetically modified loci provided here. For example, in some embodiments provided herein are methods of making non-human animals (e.g., a mouse or a rat) and ES cells that comprise (i) a homozygous null mutation in Rag1 and/or Rag2 gene (e.g., a Rag 1 and/or Rag2 gene knock-out); (ii) a homozygous null mutation in IL2rg gene (e.g., a IL2rg gene knock-out); (iii) a homozygous null mutation in the non-human animal FMS-like tyrosine kinase 3 (Flt3) gene, and (iv) a Flt3 ligand (Flt31) gene that comprises a non-human animal portion and a human portion operably linked to a Flt31 promoter. In some embodiments, provided herein are methods of making non-human animals (e.g., a mouse or a rat) and ES cells that comprise (i) a homozygous null mutation in Rag1 and/or Rag2 gene (e.g., a Rag1 and/or Rag2 gene knock-out); (ii) a homozygous null mutation in IL2rg gene (e.g., a IL2rg gene knock-out); (iii) a homozygous null mutation in the non-human animal FMS-like tyrosine kinase 3 (Flt3) gene, and (iv) a Flt3 ligand (Flt31) gene that comprises a non-human animal portion and a human portion operably linked to a Flt31 promoter, and further comprise a humanized Sirpa locus provided herein. In some embodiments, provided herein are methods of making non-human animals (e.g., a mouse or a rat) and ES cells that further comprise a humanized GM-CSF locus provided herein, and/or a humanized IL-3 locus provided herein. The exemplary methods of making genetically modified non-human animals and ES cells provided herein are described in the description, examples, and/or figures herein.

The generation of a non-human animal comprising a null mutation in the non-human animal Flt3 gene, and/or a non-human animal comprising a Flt3 ligand (Flt31) gene that comprises a non-human animal portion and a human portion operably linked to a Flt31 promoter may be accomplished using any convenient method for the making genetically modified animals, e.g., as known in the art or as described herein in Example 1.

The generation of a non-human animal comprising a nucleic acid sequence that encodes a human or humanized protein (e.g., hSIRPA, hGM-CSF, or hIL-3) may be accomplished using any convenient method for the making genetically modified animals, e.g., as known in the art or as described herein.

For example, a nucleic acid encoding the human or humanized protein (e.g., hSIRPA, hGM-CSF, and/or hIL-3) may be incorporated into a recombinant vector in a form suitable for insertion into the genome of the host cell and expression of the human protein in a non-human host cell. In various embodiments, the recombinant vector may include the one or more regulatory sequences operatively linked to the nucleic acid encoding the human protein in a manner which allows for transcription of the nucleic acid into mRNA and translation of the mRNA into the human protein, as described above. It will be understood that the design of the vector may depend on such factors as the choice of the host cell to be transfected and/or the amount of human protein to be expressed.

Any of various methods may then be used to introduce the human nucleic acid sequence into an animal cell to produce a genetically modified animal that expresses the human gene. Such techniques are well-known in the art and include, but are not limited to, pronuclear microinjection, transformation of embryonic stem cells, homologous recombination and knock-in techniques. Methods for generating genetically modified animals that can be used include, but are not limited to, those described in Sundberg and Ichiki (2006, Genetically Engineered Mice Handbook, CRC Press), Hofker and van Deursen (2002, Genetically modified Mouse Methods and Protocols, Humana Press), Joyner (2000, Gene Targeting: A Practical Approach, Oxford University Press), Turksen (2002, Embryonic stem cells: Methods and Protocols in Methods Mol Biol, Humana Press), Meyer et al. (2010, Proc. Nat. Acad. Sci. USA 107: 15022-15026), and Gibson (2004, A Primer Of Genome Science 2nd ed. Sunderland, Massachusetts: Sinauer), U.S. Pat. No. 6,586,251, Rathinam et al. (2011, Blood 118:3119-28), Willinger et al, (2011, Proc Natl Acad Sci USA, 108:2390-2395), Rongvaux et al, (2011, Proc Natl Acad Sci USA, 108:2378-83) and Valenzuela et al. (2003, Nat Biot 21:652-659).

For example, the subject genetically modified animals can be created by introducing the nucleic acid encoding the human protein into an oocyte, e.g., by microinjection, and allowing the oocyte to develop in a female foster animal. In preferred embodiments, the expression is injected into fertilized oocytes. Fertilized oocytes can be collected from superovulated females the day after mating and injected with the expression construct. The injected oocytes are either cultured overnight or transferred directly into oviducts of 0.5-day p.c. pseudopregnant females. Methods for superovulation, harvesting of oocytes, expression construct injection and embryo transfer are known in the art and described in Manipulating the Mouse Embryo (2002, A Laboratory Manual, 3rd edition, Cold Spring Harbor Laboratory Press). Offspring can be evaluated for the presence of the introduced nucleic acid by DNA analysis (e.g., PCR, Southern blot, DNA sequencing, etc.) or by protein analysis (e.g., ELISA, Western blot, etc.).

As another example, the construct comprising the nucleic acid sequence encoding the human protein may be transfected into stem cells (e.g., ES cells or iPS cells) using well-known methods, such as electroporation, calcium-phosphate precipitation, lipofection, etc. The cells can be evaluated for the presence of the introduced nucleic acid by DNA analysis (e.g., PCR, Southern blot, DNA sequencing, etc.) or by protein analysis (e.g., ELISA, Western blot, etc.). Cells determined to have incorporated the expression construct can then be introduced into preimplantation embryos. For a detailed description of methods known in the art useful for the compositions and methods of the invention, see Nagy et al, (2002, Manipulating the Mouse Embryo: A Laboratory Manual, 3rd edition, Cold Spring Harbor Laboratory Press), Nagy et al. (1990, Development 110:815-821), U.S. Pat. Nos. 7,576,259, 7,659,442, 7,294,754, and Kraus et al. (2010, Genesis 48:394-399).

Additionally, as described in some of the Examples below, a nucleic acid construct may be constructed using VELOCIGENE® genetic engineering technology (see, e.g., Valenzuela et al. (2003) High throughput engineering of the mouse genome coupled with high-resolution expression analysis, Nature Biotech. 21(6): 652-59 and U.S. Pat. No. 6,586,251), introduced into stem cells (e.g., ES cells), and correctly targeted clones determined using loss-of-allele and gain-of-allele assays (Valenzuela et al, supra); correctly targeted ES cells may be used as donor ES cells for introduction into an 8-cell stage mouse embryo using the VELOCIMOUSE® method (see, e.g., U.S. Pat. No. 7,294,754 and Poueymirou et al. 2007, F0 generation mice that are essentially fully derived from the donor gene-targeted ES cells allowing immediate phenotypic analyses Nature Biotech. 25(1):91-99). In addition, genetically modified rat ES cells and rats can be made according to US 2014/0235933 A1 (Regeneron Pharmaceuticals, Inc.), US 2014/0310828 A1 (Regeneron Pharmaceuticals, Inc.), Tong et al. (2010) Nature 467:211-215, and Tong et al. (2011) Nat Protoc. 6(6): doi:10.1038/nprot.2011.338 (all of which are incorporated herein by reference in their entireties).

In some embodiments, genetically modified founder animals can be bred to additional animals carrying one or more genetic modifications. For example, the genetically modified non-human animals (e.g., rodents, such as rats or mice) provided herein can be generated by breeding Flt3-deficient non-human animals provided herein to non-human animals comprising a Flt3 ligand (Flt31) gene that comprises a non-human animal portion and a human portion operably linked to a Flt31 promoter. These genetically modified non-human animals (e.g., rodents, such as rats or mice) can further be bred to other genetically modified non-human animals carrying other genetic modifications, which include introduction of either completely human or humanized genes, e.g., hSirpa knock-in mice, hIL-3 knock-in mice, hGM-CSF knock-in mice, and the like, or be bred to knockout animals, e.g., a non-human animal that is deficient for one or more proteins, e.g., does not express one or more of its genes, e.g., a Rag1-deficient animal, a Rag2-deficient animal, or an IL-2rg-deficient animal.

In another embodiment, stem cells, e.g., ES cells, may be generated such that they comprise several genetic modifications, e.g., humanizations or gene deletions described herein, and such stem cells may be introduced into an embryo to generate genetically modified animals with several genetic modifications.

As discussed above, in some embodiments, the genetically modified non-human animal is an immunodeficient animal. Genetically modified non-human animals that are immunodeficient and comprise one or more human or humanized proteins, e.g., hSIRPA, hIL-3, and/or hGM-CSF, may be generated using any convenient method for the generation of genetically modified animals, e.g., as known in the art or as described herein. For example, the generation of the genetically modified immunodeficient animal can be achieved by introduction of the nucleic acid encoding the human protein into an oocyte or stem cells comprising a mutant SCID gene allele or Rag1 and/or Rag2 and Il2rg null alleles that, when homozygous, will result in immunodeficiency as described in greater detail above and in the working examples herein. Mice are then generated with the modified oocyte or ES cells using, e.g., methods described herein and known in the art, and mated to produce the immunodeficient mice comprising the desired genetic modification. As another example, genetically modified non-human animals can be generated in an immunocompetent background, and crossed to an animal comprising a mutant gene allele that, when hemizygous or homozygous, will result in immunodeficiency, and the progeny mated to create an immunodeficient animal expressing the at least one human protein of interest.

In some embodiments, the genetically modified mouse is treated so as to eliminate endogenous hematopoietic cells that may exist in the mouse. In one embodiment, the treatment comprises irradiating the genetically modified mouse. In a specific embodiment, newborn genetically modified mouse pups are irradiated sublethally. In a specific embodiment, newborn pups are irradiated 2×200 cGy with a four hour interval.

Various embodiments of the invention provide genetically modified animals that include a human nucleic acid in substantially all of their cells, as well as genetically modified animals that include a human nucleic acid in some, but not all their cells. In some instances, e.g., targeted recombination, one copy of the human nucleic acid will be integrated into the genome of the genetically modified animals. In other instances, e.g., random integration, multiple copies, adjacent or distant to one another, of the human nucleic acid may be integrated into the genome of the genetically modified animals.

Thus, in some embodiments, the subject genetically modified non-human animal may be an immunodeficient animal comprising a genome that includes a nucleic acid encoding a human polypeptide operably linked to the corresponding non-human animal promoter, wherein the animal expresses the encoded human polypeptide. In other words, the subject genetically modified immunodeficient non-human animal comprises a genome that comprises a nucleic acid encoding at least one human polypeptide, wherein the nucleic acid is operably linked to the corresponding non-human promoter and a polyadenylation signal, and wherein the animal expresses the encoded human polypeptide.

Additional methods of generating genetically modified non-human animals comprising a genome that includes a nucleic acid encoding one or more human proteins, e.g., hSIRPA, hIL-3, and/or hGM-CSF, are described in U.S. Pat. No. 11,019,810 and WO 2011/044050, each of which each of which is incorporated by reference herein.

### Engraftment

In some embodiments, the subject genetically modified non-human animal is also immunodeficient. “Immunodeficient” includes deficiencies in one or more aspects of an animal's native, or endogenous, immune system, e.g., the animal is deficient for one or more types of functioning host immune cells, e.g., deficient for non-human B cell number and/or function, non-human T cell number and/or function, non-human NK cell number and/or function, etc.

One method to achieve immunodeficiency in the subject animals is sublethal irradiation. Alternatively or in addition, immunodeficiency may be achieved by any one of a number of gene mutations known in the art, any of which may be bred either alone or in combination into the subject genetically modified non-human animals of the present disclosure or which may be used as the source of stem cells into which the genetic modifications of the subject disclosure may be introduced. Non-limiting examples include X-linked SCID, associated with IL2RG gene mutations and characterized by the lymphocyte phenotype T(−) B(+) NK(−); autosomal recessive SCID associated with Jak3 gene mutations and characterized by the lymphocyte phenotype T(−) B(+) NK(−); ADA gene mutations characterized by the lymphocyte phenotype T(−) B(−) NK(−); IL-7R alpha-chain mutations characterized by the lymphocyte phenotype T(−) B(+) NK(+); CD3 delta or epsilon mutations characterized by the lymphocyte phenotype T(−) B(+) NK(+); RAG1 and RAG2 mutations characterized by the lymphocyte phenotype T(−) B(−) NK(+); Artemis gene mutations characterized by the lymphocyte phenotype T(−) B(−) NK(+), CD45 gene mutations characterized by the lymphocyte phenotype T(−) B(+) NK(+); and Prkdcscld mutations characterized by the lymphocyte phenotype T(−), B(−). As such, in some embodiments, the genetically modified immunodeficient non-human animal has one or more deficiencies selected from an IL2 receptor gamma chain deficiency, a Jak3 deficiency, an ADA deficiency, an IL7R deficiency, a CD3 deficiency, a RAG1 and/or RAG2 deficiency, an Artemis deficiency, a CD45 deficiency, and a Prkdc deficiency. In one embodiment, the immunodeficiency is achieved by gene mutation or deletion in Rag1 and/or Rag2 and Il2rg genes. These and other animal models of immunodeficiency will be known to the ordinarily skilled artisan, any of which may be used to generate immunodeficient animals of the present disclosure.

In some embodiments, genetically modified non-human animals in accordance with the invention find use as recipients of human hematopoietic cells that are capable of developing human immune cells from engrafted human hematopoietic cells. As such, in some aspects of the invention, the subject genetically modified animal is a genetically modified, immunodeficient, non-human animal that is engrafted with human hematopoietic cells.

Any source of human hematopoietic cells, human hematopoietic stem cells (HSCs) and/or hematopoietic stem progenitor cells (HSPC) as known in the art or described herein may be transplanted into the genetically modified immunodeficient non-human animals of the present disclosure. One suitable source of human hematopoietic cells known in the art is human umbilical cord blood cells, in particular CD34-positive (CD34+) cells. Another source of human hematopoietic cells is human fetal liver. Another source is human bone marrow. Also encompassed are induced pluripotent stem cells (iPSC) and induced hematopoietic stem cells (iHSC) produced by the de-differentiation of somatic cells, e.g., by methods known in the art. Methods for the transplantation of human cells into non-human animals are well-described in the art and elsewhere herein, any of which may be employed by the ordinarily skilled artisan to arrive at the subject genetically modified engrafted non-human animals.

Cell populations of particular interest include those that comprise hematopoietic stem or progenitor cells, which will contribute to or reconstitute the hematopoietic system of the genetically modified non-human animals, for example, peripheral blood leukocytes, fetal liver cells, fetal bone, fetal thymus, fetal lymph nodes, vascularized skin, artery segments, and purified hematopoietic stem cells, e.g., mobilized HSCs or cord blood HSCs.

Cells may be from any mammalian species, e.g., murine, rodent, canine, feline, equine, bovine, ovine, primate, human, etc. In one embodiment, the cells are human cells. Cells may be from established cell lines or they may be primary cells, where “primary cells”, “primary cell lines”, and “primary cultures” are used interchangeably herein to refer to cells and cells cultures that have been derived from a subject and allowed to grow in vitro for a limited number of passages, i.e., splittings, of the culture. For example, primary cultures are cultures that may have been passaged 0 times, 1 time, 2 times, 4 times, 5 times, 10 times, or 15 times, but not enough times go through the crisis stage. Typically, the primary cell lines of the present disclosure are maintained for fewer than 10 passages in vitro.

If the cells are primary cells, they may be harvested from an individual by any convenient method. For example, cells, e.g., blood cells, e.g., leukocytes, may be harvested by apheresis, leukocytapheresis, density gradient separation, etc. As another example, cells, e.g., skin, muscle, bone marrow, spleen, liver, pancreas, lung, intestine, stomach tissue, etc. may be harvested by biopsy. An appropriate solution may be used for dispersion or suspension of the harvested cells. Such solution will generally be a balanced salt solution, e.g., normal saline, PBS, Hank's balanced salt solution, etc., conveniently supplemented with fetal calf serum or other naturally occurring factors, in conjunction with an acceptable buffer at low concentration, generally from 5-25 mM. Convenient buffers include HEPES, phosphate buffers, lactate buffers, etc.

In some instances, a heterogeneous population of cells will be transplanted into the genetically modified non-human animals. In other instances, a population of cells that is enriched for a particular type of cell, e.g., a progenitor cell, e.g., a hematopoietic progenitor cell, will be engrafted into the genetically modified non-human animals. Enrichment of a cell population of interest may be by any convenient separation technique. For example, the cells of interest may be enriched by culturing methods. In such culturing methods, particular growth factors and nutrients are typically added to a culture that promote the survival and/or proliferation of one cell population over others. Other culture conditions that affect survival and/or proliferation include growth on adherent or non-adherent substrates, culturing for particular lengths of time, etc. Such culture conditions are well known in the art. As another example, cells of interest may be enriched for by separation the cells of interest from the initial population by affinity separation techniques. Techniques for affinity separation may include magnetic separation using magnetic beads coated with an affinity reagent, affinity chromatography, “panning” with an affinity reagent attached to a solid matrix, e.g., plate, cytotoxic agents joined to an affinity reagent or used in conjunction with an affinity reagent, e.g., complement and cytotoxins, or other convenient technique. Techniques providing accurate separation include fluorescence activated cell sorters, which can have varying degrees of sophistication, such as multiple color channels, low angle and obtuse light scattering detecting channels, impedance channels, etc. The cells may be selected against dead cells by employing dyes associated with dead cells (e.g., propidium iodide). Any technique may be employed which is not unduly detrimental to the viability of the cells of interest.

For example, using affinity separation techniques, cells that are not the cells of interest for transplantation may be depleted from the population by contacting the population with affinity reagents that specifically recognize and selectively bind markers that are not expressed on the cells of interest. For example, to enrich for a population of hematopoietic progenitor cells, one might deplete cells expressing mature hematopoietic cell markers. Additionally or alternatively, positive selection and separation may be performed using by contacting the population with affinity reagents that specifically recognize and selectively bind markers associated with hematopoietic progenitor cells, e.g., CD34, CD133, etc. By “selectively bind” is meant that the molecule binds preferentially to the target of interest or binds with greater affinity to the target than to other molecules. For example, an antibody will bind to a molecule comprising an epitope for which it is specific and not to unrelated epitopes. In some embodiments, the affinity reagent may be an antibody, i.e., an antibody that is specific for CD34, CD133, etc. In some embodiments, the affinity reagent may be a specific receptor or ligand for CD34, CD133, etc., e.g., a peptide ligand and receptor; effector and receptor molecules, a T-cell receptor specific for CD34, CD133, etc., and the like. In some embodiments, multiple affinity reagents specific for the marker of interest may be used.

Antibodies and T cell receptors that find use as affinity reagents may be monoclonal or polyclonal, and may be produced by transgenic animals, immunized animals, immortalized human or animal B-cells, cells transfected with DNA vectors encoding the antibody or T cell receptor, etc. The details of the preparation of antibodies and their suitability for use as specific binding members are well-known to those skilled in the art. Of particular interest is the use of labeled antibodies as affinity reagents. Conveniently, these antibodies are conjugated with a label for use in separation. Labels include magnetic beads, which allow for direct separation; biotin, which can be removed with avidin or streptavidin bound to a support; fluorochromes, which can be used with a fluorescence activated cell sorter; or the like, to allow for ease of separation of the particular cell type. Fluorochromes that find use include phycobiliproteins, e.g., phycoerythrin and allophycocyanins, fluorescein and Texas red. Frequently each antibody is labeled with a different fluorochrome, to permit independent sorting for each marker.

The initial population of cells are contacted with the affinity reagent(s) and incubated for a period of time sufficient to bind the available cell surface antigens. The incubation will usually be at least about 5 minutes and usually less than about 60 minutes. It is desirable to have a sufficient concentration of antibodies in the reaction mixture, such that the efficiency of the separation is not limited by lack of antibody. The appropriate concentration is determined by titration, but will typically be a dilution of antibody into the volume of the cell suspension that is about 1:50 (i.e., 1 part antibody to 50 parts reaction volume), about 1:100, about 1:150, about 1:200, about 1:250, about 1:500, about 1:1000, about 1:2000, or about 1:5000. The medium in which the cells are suspended will be any medium that maintains the viability of the cells. A preferred medium is phosphate buffered saline containing from 0.1 to 0.5% BSA or 1-4% goat serum. Various media are commercially available and may be used according to the nature of the cells, including Dulbecco's Modified Eagle Medium (dMEM), Hank's Basic Salt Solution (HBSS), Dulbecco's phosphate buffered saline (dPBS), RPMI, Iscove's medium, PBS with 5 mM EDTA, etc., frequently supplemented with fetal calf serum, BSA, HSA, goat serum etc.

The cells in the contacted population that become labeled by the affinity reagent are selected for by any convenient affinity separation technique, e.g., as described above or as known in the art. Following separation, the separated cells may be collected in any appropriate medium that maintains the viability of the cells, usually having a cushion of serum at the bottom of the collection tube. Various media are commercially available and may be used according to the nature of the cells, including dMEM, HBSS, dPBS, RPMI, Iscove's medium, etc., frequently supplemented with fetal calf serum.

Compositions highly enriched for a cell type of interest, e.g., hematopoietic cells, are achieved in this manner. The cells will be about 70%, about 75%, about 80%, about 85% about 90% or more of the cell composition, about 95% or more of the enriched cell composition, and will preferably be about 95% or more of the enriched cell composition. In other words, the composition will be a substantially pure composition of cells of interest.

The cells to be transplanted into the genetically modified non-human animals, be they a heterogeneous population of cells or an enriched population of cells, may be transplanted immediately. Alternatively, the cells may be frozen at liquid nitrogen temperatures and stored for long periods of time, being thawed and capable of being reused. In such cases, the cells will usually be frozen in 10% DMSO, 50% serum, 40% buffered medium, or some other such solution as is commonly used in the art to preserve cells at such freezing temperatures, and thawed in a manner as commonly known in the art for thawing frozen cultured cells. Additionally or alternatively, the cells may be cultured in vitro under various culture conditions. Culture medium may be liquid or semi-solid, e.g., containing agar, methylcellulose, etc. The cell population may be conveniently suspended in an appropriate nutrient medium, such as Iscove's modified DMEM or RPMI-1640, normally supplemented with fetal calf serum (about 5-10%), L-glutamine, a thiol, particularly 2-mercaptoethanol, and antibiotics, e.g., penicillin and streptomycin. The culture may contain growth factors to which the cells are responsive. Growth factors, as defined herein, are molecules capable of promoting survival, growth and/or differentiation of cells, either in culture or in the intact tissue, through specific effects on a transmembrane receptor. Growth factors include polypeptides and non-polypeptide factors.

The cells may be genetically modified prior to transplanting to the genetically modified non-human animals, e.g., to provide a selectable or traceable marker, to induce a genetic defect in the cells (e.g., for disease modeling), to repair of a genetic defect or ectopically express a gene in the cells (e.g., to determine if such modifications will impact the course of a disease), etc. Cells may be genetically modified by transfection or transduction with a suitable vector, homologous recombination, or other appropriate technique, so that they express a gene of interest, or with an antisense mRNA, siRNA or ribozymes to block expression of an undesired gene. Various techniques are known in the art for the introduction of nucleic acids into target cells. To prove that one has genetically modified the cells, various techniques may be employed. The genome of the cells may be restricted and used with or without amplification. The polymerase chain reaction; gel electrophoresis; restriction analysis; Southern, Northern, and Western blots; sequencing; or the like, may all be employed. General methods in molecular and cellular biochemistry for these and other purposes disclosed in this application can be found in such standard textbooks as Molecular Cloning: A Laboratory Manual, 3rd Ed. (Sambrook et al., Cold Spring Harbor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference. Reagents, cloning vectors, and kits for genetic manipulation referred to in this disclosure are available from commercial vendors such as BioRad, Stratagene, Invitrogen, Sigma-Aldrich, and ClonTech.

The cells may be transplanted in the genetically modified non-human animals by any convenient method, including, for example, intra-hepatic injection, tail-vein injection, retro-orbital injection, and the like. Typically, about 0.5×10⁵-2×10⁶ pluripotent or progenitor cells are transplanted, e.g., about 1×10⁵-1×10⁶ cells, or about 2×10⁵-5×10⁵ cells. In some instances, the mouse is sublethally irradiated prior to transplanting the human cells. In other words, the mouse is exposed to a sublethal dose of radiation, e.g., as described in the examples section below and as well-known in the art. The engrafted genetically modified non-human animal is then maintained under laboratory animal husbandry conditions for at least 1 week, e.g., 1 week or more, or two weeks or more, sometimes 4 weeks or more, and in some instances 6 weeks or more, to allow sufficient reconstitution of the immune system with the engrafted cells.

In some embodiments, the transplanted human hematopoietic cells give rise in the genetically modified non-human animal to one or more engrafted human cells selected from a human CD34-positive cell, a human hematopoietic stem cell, a human hematopoietic cell, a myeloid progenitor cell, an erythroid progenitor cell, a myeloid cell, a dendritic cell, a monocyte, a neutrophil, a mast cell, an erythrocyte, and a combination thereof. In one embodiment, the human cell is present at 4 months, 5 months, 6 months, 7 months, 8 months, 9 months, 10 months, 11 months, or 12 months after engraftment. In a specific embodiment, the human cells comprise human dendritic cells.

In some embodiments, the transplanted human hematopoietic cells give rise in the genetically modified non-human animal to an engrafted human hemato-lymphoid system that comprises human hematopoietic stem and progenitor cells, human myeloid progenitor cells, human myeloid cells, human dendritic cells, human monocytes, human granulocytes, human neutrophils, human mast cells, human erythrocytes, human thymocytes, human T cells, human B cells, and human platelets. In one embodiment, the human hemato-lymphoid system is present at 4 months, 5 months, 6 months, 7 months, 8 months, 9 months, 10 months, 11 months, or 12 months after engraftment. In a specific embodiment, the human hemato-lymphoid system comprises the human dendritic cells.

In some aspects, the genetically modified non-human animal provided herein is engrafted with human hematopoietic cells comprising a disease-specific mutation.

### Nonlimiting Applications of Genetically Modified Engrafted Mice

The genetically modified non-human animals of the present disclosure find many uses in the art. For example, engrafted genetically modified animals of the present disclosure are useful for studying the function of human dendritic cells, e.g., the function of human myeloid DCs or human plasmacytoid DCs. As another example, engrafted genetically modified mice of the present disclosure provide a useful system for screening candidate agents for desired activities in vivo, for example, to identify agents that are able to modulate (i.e., promote or suppress) the function of human dendritic cells (e.g., human myeloid DCs or human plasmacytoid DCs), e.g., in a healthy or a diseased state. For example, engrafted genetically modified mice of the present disclosure can be used to identify novel therapeutics. As another example, engrafted genetically modified animals of the present disclosure provide a useful system for predicting the responsiveness of an individual to a disease therapy, e.g., by providing an in vivo platform for screening the responsiveness of an individual's immune system to an agent, e.g., a therapeutic agent, to predict the responsiveness of an individual to that agent. In some embodiments, because myeloid DCs (mDCs) are important for cross-priming and activation of T cell, engrafted genetically modified animals of the present disclosure provide a useful model for testing potential immunotherapies that stimulate T cell response by targeting mDCs. For example, engrafted genetically modified animals of the present disclosure provide a useful system for assessing therapeutic efficacy of a drug to stimulate a T cell response to a tumor cell or a pathogen, or for identifying a drug that stimulates a T cell response to a tumor cell or a pathogen. In some embodiments, because plasmacytoid DCs (pDCs) are critical type I IFN producers in viral infection and have also been implicated in autoimmune diseases such as lupus, engrafted genetically modified animals of the present disclosure provide a useful model for testing therapies for viral infection or autoimmune diseases by targeting pDCs. For example, engrafted genetically modified animals of the present disclosure provide a useful system for assessing therapeutic efficacy of a drug in treating viral infection or an autoimmune disease, or for identifying a drug that treat viral infection or an autoimmune disease.

As one non-limiting example, engrafted genetically modified mice of the present disclosure find use in the generation of mouse models of an autoimmune disease (e.g., systemic lupus erythematosus, systemic sclerosis, sjogren's syndrome, polymyositis, collagen-induced arthritis, or dermatomyositis). Such mouse models of the autoimmune disease will be useful in both research, e.g., to better understand the progression of the autoimmune disease in humans, and in drug discovery, e.g., to identify candidate agents that treat the autoimmune disease.

Engrafted genetically modified animals of the present disclosure find use in screening candidate agents to identify those that will treat an autoimmune disease. The terms “treatment”, “treating” and the like are used herein to generally include obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein include any treatment of a disease in a mammal, and includes: (a) preventing the disease from occurring in a subject which may be predisposed to the disease but has not yet been diagnosed as having it; (b) inhibiting the disease, i.e., arresting its development; or (c) relieving the disease, i.e., causing regression of the disease. The terms “individual,” “subject,” “host,” and “patient,” are used interchangeably herein and include any mammalian subject for whom diagnosis, treatment, or therapy is desired, particularly humans.

As another non-limiting example, engrafted genetically modified mice of the present disclosure find use in the generation of mouse models for assessing therapeutic efficacy of a drug to stimulate a T cell response to a target cell (e.g., a tumor cell, a virally-infected cell, a bacterially-infected cell, a bacterial cell, a fungal cell, and a parasitic cell). Such mouse models will be useful in drug discovery, e.g., to identify candidate agents that to stimulate a T cell response to a target cell.

In screening assays for biologically active agents, a human hematopoietic cell-engrafted genetically modified non-human animal of the present disclosure, is contacted with a candidate agent of interest and the effect of the candidate agent is assessed by monitoring one or more output parameters. These output parameters may be reflective of the function of the human dendritic cell, e.g., phagocytosis, cytokine production, cross-presentation of exogenous antigens, and activation of cytotoxic CD8+ T cell lymphocytes (CTLs); or of the T cell response to the target cell in the rodent, by methods that are well known in the art. Alternatively or additionally, the output parameters may be reflective of the effect of the agent on an autoimmune disease in the human hematopoietic cell-engrafted genetically modified non-human animal of the present disclosure.

Candidate agents for screening or methods of the present disclosure may include, for examples, organic molecules (e.g., small molecule inhibitors), nucleic acids (e.g., RNA interfering agents, oligonucleotides, or nucleic acids that encode polypeptides), peptides, peptidomimetic inhibitors, aptamers, antibodies, intrabodies, etc. The “RNA interfering agent” used herein may be a small interfering RNA (siRNA), CRISPR RNA (crRNA), CRISPR guide RNA (gRNA), a small hairpin RNA (shRNA), a microRNA (miRNA), or a piwi-interacting RNA (piRNA).

Candidate agents are screened for biological activity by administering the agent to at least one and usually a plurality of samples, sometimes in conjunction with samples lacking the agent. The change in parameters in response to the agent is measured, and the result evaluated by comparison to reference samples, e.g., in the presence and absence of the agent, obtained with other agents, etc. In instances in which a screen is being performed to identify candidate agents that will prevent, mitigate or reverse the effects of a pathogen or a non-pathogenic immune stimulation, the screen is typically performed in the presence of the pathogenic agent or the non-pathogenic immune stimulation, where the pathogenic agent or the non-pathogenic immune stimulation is added at the time most appropriate to the results to be determined. For example, in cases in which the protective/preventative ability of the candidate agent is tested, the candidate agent may be added before the pathogen or the non-pathogenic immune stimulation, simultaneously with the pathogen or the non-pathogenic immune stimulation, or subsequent to infection by the pathogen or to the non-pathogenic immune stimulation. As another example, in cases in which the ability of the candidate agent to reverse the effects of a pathogen or a non-pathogenic immune stimulation is tested, the candidate agent may be added subsequent to infection with the pathogen or to the non-pathogenic immune stimulation. As mentioned above, in some instances, the “sample” is a genetically modified non-human animal that has been engrafted with cells, e.g., the candidate agent is provided to a genetically modified non-human animal that has been engrafted with human hematopoietic cells. In some instances, the “sample” is the human hematopoietic cells to be engrafted, i.e., the candidate agent is provided to cells, e.g., human dendritic cells, etc., prior to engraftment into the immunodeficient genetically modified animal.

If the candidate agent is to be administered directly to the engrafted genetically modified animal, the agent may be administered by any of a number of well-known methods in the art for the administration of peptides, small molecules and nucleic acids to mice. For example, the agent may be administered orally, mucosally, topically, intradermally, or by injection, e.g., intraperitoneal, subcutaneous, intramuscular, or intravenous injection, and the like. The agent may be administered in a buffer, or it may be incorporated into any of a variety of formulations, e.g., by combination with appropriate pharmaceutically acceptable vehicle. “Pharmaceutically acceptable vehicles” may be vehicles approved by a regulatory agency of the Federal or a state government or listed in the U.S. Pharmacopeia or other generally recognized pharmacopeia for use in mammals, such as humans. The term “vehicle” refers to a diluent, adjuvant, excipient, or carrier with which a compound of the invention is formulated for administration to a mammal. Such pharmaceutical vehicles can be lipids, e.g., liposomes, e.g., liposome dendrimers; liquids, such as water and oils, including those of petroleum, animal, vegetable or synthetic origin, such as peanut oil, soybean oil, mineral oil, sesame oil and the like, saline; gum acacia, gelatin, starch paste, talc, keratin, colloidal silica, urea, and the like. In addition, auxiliary, stabilizing, thickening, lubricating and coloring agents may be used. Pharmaceutical compositions may be formulated into preparations in solid, semi-solid, liquid or gaseous forms, such as tablets, capsules, powders, granules, ointments, solutions, suppositories, injections, inhalants, gels, microspheres, and aerosols. The agent may be systemic after administration or may be localized by the use of regional administration, intramural administration, or use of an implant that acts to retain the active dose at the site of implantation. The active agent may be formulated for immediate activity or it may be formulated for sustained release. If the agent(s) are provided to cells prior to engraftment, the agents are conveniently added in solution, or readily soluble form, to the medium of cells in culture. The agents may be added in a flow-through system, as a stream, intermittent or continuous, or alternatively, adding a bolus of the compound, singly or incrementally, to an otherwise static solution. In a flow-through system, two fluids are used, where one is a physiologically neutral solution, and the other is the same solution with the test compound added. The first fluid is passed over the cells, followed by the second. In a single solution method, a bolus of the test compound is added to the volume of medium surrounding the cells. The overall concentrations of the components of the culture medium should not change significantly with the addition of the bolus, or between the two solutions in a flow through method.

A plurality of assays may be run in parallel with different agent concentrations to obtain a differential response to the various concentrations. As known in the art, determining the effective concentration of an agent typically uses a range of concentrations resulting from 1:10, or other log scale, dilutions. The concentrations may be further refined with a second series of dilutions, if necessary. Typically, one of these concentrations serves as a negative control, i.e., at zero concentration or below the level of detection of the agent or at or below the concentration of agent that does not give a detectable change in the phenotype.

An analysis of the response of cells in the engrafted genetically modified animal to the candidate agent may be performed at any time following treatment with the agent. For example, the cells may be analyzed 1, 2, or 3 days, sometimes 4, 5, or 6 days, sometimes 8, 9, or 10 days, sometimes 14 days, sometimes 21 days, sometimes 28 days, sometimes 1 month or more after contact with the candidate agent, e.g., 2 months, 4 months, 6 months or more. In some embodiments, the analysis comprises analysis at multiple time points. The selection of the time point(s) for analysis will be based upon the type of analysis to be performed, as will be readily understood by the ordinarily skilled artisan.

The analysis may comprise measuring any of the parameters described herein or known in the art for measuring cell viability, cell proliferation, cell identity, cell morphology, and cell function, particularly as they may pertain to cells of the immune cells. For example, flow cytometry may be used to determine the total number of hematopoietic cells or the number of cells of a particular hematopoietic cell type. Histochemistry or immunohistochemistry may be performed to determine the apoptotic state of the cells, e.g., terminal deoxynucleotidyl transferase dUTP nick end labeling (TUNEL) to measure DNA fragmentation, or immunohistochemistry to detect Annexin V binding to phosphatidylserine on the cell surface. Flow cytometry may also be employed to assess the proportions of differentiated cells and differentiated cell types, e.g., to determine the ability of hematopoietic cells to survive and/or differentiate in the presence of agent. ELISAs, Westerns, and Northern blots may be performed to determine the levels of cytokines, chemokines, immunoglobulins, etc. expressed in the engrafted genetically modified mice, e.g., to assess the function of the engrafted cells, to assess the functional of human dendritic cells, etc. In vivo assays to test the function of immune cells, as well as assays relevant to particular diseases or disorders of interest such as autoimmune diseases, e.g., systemic lupus erythematosus, systemic sclerosis, sjogren's syndrome, polymyositis, collagen-induced arthritis, or dermatomyositis, etc. may also be performed. See, e.g., Current Protocols in Immunology (Richard Coico, ed. John Wiley & Sons, Inc. 2012) and Immunology Methods Manual (I. Lefkovits ed., Academic Press 1997), the disclosures of which are incorporated herein by reference.

Other examples of uses for the subject mice are provided elsewhere herein. Additional applications of the genetically modified and engrafted mice described in this disclosure will be apparent to those skilled in the art upon reading this disclosure.

### ADDITIONAL EXEMPLARY EMBODIMENTS

In exemplary embodiment 1, provided herein is a genetically modified rodent, comprising: (i) a homozygous null mutation in Rag2 gene; (ii) a homozygous null mutation in IL2rg gene; (iii) a homozygous null mutation in the rodent FMS-like tyrosine kinase 3 (Flt3) gene; and (iv) a Flt3 ligand (Flt31) gene that comprises a rodent portion and a human portion operably linked to a Flt31 promoter.

In exemplary embodiment 2, provided herein is a genetically modified rodent of embodiment 1 comprising a homozygous null mutation in Rag1 gene.

In exemplary embodiment 3, provided herein is a genetically modified rodent of embodiment 1 or 2, wherein the null mutation in the rodent Flt3 gene comprises an insertion, a deletion, and/or a substitution in the endogenous Flt3 gene.

In exemplary embodiment 4, provided herein is a genetically modified rodent of embodiment 3, wherein the null mutation in the rodent Flt3 gene is a deletion of the full Flt3 endogenous coding sequence.

In exemplary embodiment 5, provided herein is a genetically modified rodent of embodiment 4, wherein the genetically modified rodent is a mouse, and the mouse comprises a homozygous deletion of nucleic acid sequence between coordinates chr5: 147331171-147400265 (GRCm38 assembly).

In exemplary embodiment 6, provided herein is a genetically modified rodent of any one of embodiments 1-5, wherein the rodent portion of the Flt31 gene comprises exon 1, a non-coding portion of exon 2, and exons downstream of exon 6 of a rodent Flt31 gene.

In exemplary embodiment 7, provided herein is a genetically modified rodent of embodiment 6, wherein the exon 1, the non-coding portion of exon 2, and the exons downstream of exon 6 of the rodent Flt31 gene are at least 90%, at least 95%, or 100% identical to the corresponding exon 1, non-coding portion of exon 2, and exons downstream of exon 6 of a rodent Flt31 gene shown in Table 1A.

In exemplary embodiment 8, provided herein is a genetically modified rodent of any one of embodiments 1-7, wherein the human portion of the Flt31 gene comprises a signal peptide-coding portion of exon 2, and exons 3-6 of a human FLT3L gene.

In exemplary embodiment 9, provided herein is a genetically modified rodent of embodiment 8, wherein the signal peptide-coding portion of exon 2, and exons 3-6 of the human FLT3L gene are at least 50%, at least 60%, at least 70%, at least 80%, at least 90%, at least 95%, or 100% identical to the corresponding signal peptide-coding portion of exon 2, and exons 3-6 of a human FLT3L gene shown in Table 1B.

In exemplary embodiment 10, provided herein is a genetically modified rodent of any one of embodiments 1-9, wherein the Flt31 gene comprises rodent exon 1, rodent non-coding portion of exon 2, human signal peptide-coding portion of exon 2, human exons 3-6, and rodent exons 7-9.

In exemplary embodiment 11, provided herein is a genetically modified rodent of any one of embodiments 1-10, wherein the Flt31 gene encodes a chimeric membrane bound FLT3L comprising a signal peptide and cytokine-like core domain of a human FLT3L polypeptide, and a C-terminal portion of a rodent Flt31 polypeptide.

In exemplary embodiment 12, provided herein is a genetically modified rodent of embodiment 11, wherein the C-terminal portion of the rodent Flt31 polypeptide comprises a rodent stalk region, a rodent transmembrane domain, and a rodent cytoplasmic tail as shown in FIG. 1E.

In exemplary embodiment 13, provided herein is a genetically modified rodent of embodiment 11 or 12, wherein the C-terminal portion of the rodent Flt31 polypeptide has an amino acid sequence that is at least 70%, at least 80%, at least 90%, at least 95%, or 100% identical to a C-terminal portion of a rodent Flt31 polypeptide shown in Table 2.

In exemplary embodiment 14, provided herein is a genetically modified rodent of any one of embodiments 1-13, wherein the rodent expresses both soluble and membrane-bound forms of the Flt31 polypeptide.

In exemplary embodiment 15, provided herein is a genetically modified rodent of embodiment 14, wherein the soluble and membrane-bound forms of the Flt31 polypeptide comprise a signal peptide and a cytokine-like core domain of a human FLT3L polypeptide.

In exemplary embodiment 16, provided herein is a genetically modified rodent of any one of embodiments 11-15, wherein the signal peptide of the human FLT3L polypeptide comprises amino acids corresponding to residues 1-26 of a human FLT3L polypeptide.

In exemplary embodiment 17, provided herein is a genetically modified rodent of any one of embodiments 11-16, wherein the signal peptide of the human FLT3L polypeptide comprises an amino acid sequence that is at least 70%, at least 80%, at least 90%, at least 95%, or 100% identical to a corresponding amino acid sequence of a signal peptide of a human FLT3L polypeptide that appears in Table 2.

In exemplary embodiment 18, provided herein is a genetically modified rodent of any one of embodiments 11-17, wherein the cytokine-like core domain of the human FLT3L polypeptide comprises amino acids corresponding to residues 27-159 of a human FLT3L polypeptide.

In exemplary embodiment 19, provided herein is a genetically modified rodent of any one of embodiments 11-18, wherein the cytokine-like core domain of the human FLT3L polypeptide comprises an amino acid sequence that is at least 70%, at least 80%, at least 90%, at least 95%, or 100% identical to a corresponding amino acid sequence of cytokine-like core domains of a human FLT3L polypeptide that appears in Table 2.

In exemplary embodiment 20, provided herein is a genetically modified rodent of any one of embodiments 1-19, wherein the genetically modified rodent is heterozygous for the Flt3 ligand (Flt31) gene that comprises a rodent portion and a human portion.

In exemplary embodiment 21, provided herein is a genetically modified rodent of any one of embodiments 1-19, wherein the genetically modified rodent is homozygous for the Flt3 ligand (Flt31) gene that comprises a rodent portion and a human portion.

In exemplary embodiment 22, provided herein is a genetically modified rodent of any one of embodiments 1-21, wherein the rodent portion of the Flt31 gene is an endogenous rodent portion of the Flt31 gene and the rodent Flt31 gene is an endogenous rodent gene.

In exemplary embodiment 23, provided herein is a genetically modified rodent of any one of embodiments 1-22, wherein the rodent Flt31 polypeptide is an endogenous rodent Flt31 polypeptide.

In exemplary embodiment 24, provided herein is a genetically modified rodent of any one of embodiments 1-23, wherein the Flt31 promoter is a rodent promoter.

In exemplary embodiment 25, provided herein is a genetically modified rodent of embodiment 23 or 24, wherein the Flt31 promoter is an endogenous rodent promoter.

In exemplary embodiment 26, provided herein is a genetically modified rodent of any one of embodiments 23-25, wherein the Flt31 promoter is at the endogenous rodent gene locus.

In exemplary embodiment 27, provided herein is a genetically modified rodent of any one of embodiments 1-26, wherein the genetically modified rodent expresses a human or humanized SIRPA polypeptide encoded by a nucleic acid operably linked to a Sirpa promoter.

In exemplary embodiment 28, provided herein is a genetically modified rodent of embodiment 27, wherein the genetically modified rodent further comprises a Sirpa gene that encodes a Sirpa polypeptide comprising an extracellular portion of a human SIRPA polypeptide and an intracellular portion of a rodent Sirpa polypeptide, wherein the Sirpa gene is operably linked to a Sirpa promoter.

In exemplary embodiment 29, provided herein is a genetically modified rodent of embodiment 28, wherein the Sirpa gene comprises exons 1, 5, 6, 7 and 8 of a rodent Sirpa gene and exons 2-4 of a human SIRPA gene.

In exemplary embodiment 30, provided herein is a genetically modified rodent of embodiment 28 or 29, wherein the genetically modified rodent expresses a Sirpa polypeptide comprising an extracellular portion of a human SIRPA polypeptide and an intracellular portion of a rodent Sirpa polypeptide.

In exemplary embodiment 31, provided herein is a genetically modified rodent of any one of embodiments 28-30, wherein the rodent Sirpa polypeptide is an endogenous rodent Sirpa polypeptide, and/or the rodent Sirpa gene is an endogenous rodent gene.

In exemplary embodiment 32, provided herein is a genetically modified rodent of embodiment 27, wherein the genetically modified rodent expresses a human SIRPA polypeptide encoded by a nucleic acid operably linked to a Sirpa promoter.

In exemplary embodiment 33, provided herein is a genetically modified rodent of any one of embodiments 27-32, wherein the genetically modified rodent further expresses a human GM-CSF protein encoded by a nucleic acid operably linked to a GM-CSF promoter and/or a human IL3 protein encoded by a nucleic acid operably linked to a IL3 promoter.

In exemplary embodiment 34, provided herein is a genetically modified rodent of any one of embodiments 27-33, wherein the SIRPα promoter, the GM-CSF promoter, and/or the IL3 promoter is a rodent promoter.

In exemplary embodiment 35, provided herein is a genetically modified rodent of embodiment 34, wherein the SIRPα promoter, the GM-CSF promoter, and/or the IL3 promoter is an endogenous rodent promoter.

In exemplary embodiment 36, provided herein is a genetically modified rodent of embodiment 34 or 35, wherein the SIRPα promoter, the GM-CSF promoter, and/or the IL3 promoter is at the corresponding endogenous rodent gene locus.

In exemplary embodiment 37, provided herein is a genetically modified rodent of any one of embodiments 27-36, comprising a null mutation in at least one corresponding rodent gene at the corresponding rodent gene locus.

In exemplary embodiment 38, provided herein is a genetically modified rodent of any one of embodiments 27-37, wherein the genetically modified rodent is heterozygous for at least one allele comprising the nucleic acid sequence that encodes the human or humanized protein.

In exemplary embodiment 39, provided herein is a genetically modified rodent of any one of embodiments 27-38, wherein the genetically modified rodent is homozygous for at least one allele comprising the nucleic acid sequence that encodes the human or humanized protein.

In exemplary embodiment 40, provided herein is a genetically modified rodent of any one of embodiments 1-39, further comprising an engraftment of human hematopoietic cells.

In exemplary embodiment 41, provided herein is a genetically modified rodent of embodiment 40, wherein human hematopoietic cells comprise one or more cells selected from the group consisting of a human CD34-positive cell, a human hematopoietic stem cell, a human hematopoietic progenitor cell, a human dendritic cell precursor cell, and a human dendritic cell.

In exemplary embodiment 42, provided herein is a genetically modified rodent of embodiment 40 or 41, wherein the genetically modified rodent comprises human dendritic cells.

In exemplary embodiment 43, provided herein is a genetically modified rodent of any one of embodiments 1-42, wherein an autoimmune disease is induced or established in the genetically modified rodent.

In exemplary embodiment 44, provided herein is a genetically modified rodent of embodiment 43, wherein the autoimmune disease is systemic lupus erythematosus, systemic sclerosis, sjogren's syndrome, polymyositis, collagen-induced arthritis, or dermatomyositis.

In exemplary embodiment 45, provided herein is a genetically modified rodent of any one of embodiments 1-44, wherein the rodent is a mouse or a rat.

In exemplary embodiment 46, provided herein is a genetically modified rodent of embodiment 45, wherein the rodent is a mouse.

In exemplary embodiment 47, provided herein is a method of identifying an agent that modulates a function of a human dendritic cell, comprising: a. administering the candidate agent to a genetically modified rodent of any one of embodiments 40-46; and b. determining whether the candidate agent modulates the function of the human dendritic cell in the rodent.

In exemplary embodiment 48, provided herein is a method of embodiment 47, wherein the human dendritic cell is a myeloid dendritic cell (mDC) or a plasmacytoid dendritic cell (pDC).

In exemplary embodiment 49, provided herein is a method of embodiment 47 or 48, wherein the function of the human dendritic cell is selected from a group consisting of phagocytosis, cytokine production, cross-presentation of exogenous antigens, and activation of cytotoxic CD8+ T cell lymphocytes (CTLs).

In exemplary embodiment 50, provided herein is a method for assessing therapeutic efficacy of a drug to stimulate a T cell response to a target cell, the method comprising: a. administering a drug candidate to a genetically modified rodent of any one of embodiments 40-42, wherein the genetically modified rodent comprises the target cell; and b. measuring the T cell response to the target cell in the rodent to assess the therapeutic efficacy of the drug candidate.

In exemplary embodiment 51, provided herein is a method of embodiment 50, wherein the target cell is selected from the group consisting of a tumor cell, a virally-infected cell, a bacterially-infected cell, a bacterial cell, a fungal cell, and a parasitic cell.

In exemplary embodiment 52, provided herein is a method for assessing therapeutic efficacy of a drug in treating an autoimmune disease, the method comprising: a. administering the candidate agent to a genetically modified rodent of embodiment 43 or 44; and b. determining whether the agent treats the autoimmune disease in the rodent.

In exemplary embodiment 53, provided herein is a genetically modified rodent cell, comprising: (i) a homozygous null mutation in Rag2 gene; (ii) a homozygous null mutation in IL2rg gene; (iii) a homozygous null mutation in the rodent FMS-like tyrosine kinase 3 (Flt3) gene; and (iv) a Flt3 ligand (Flt31) gene that comprises a rodent portion and a human portion operably linked to a Flt31 promoter.

In exemplary embodiment 54, provided herein is a genetically modified rodent cell of embodiment 53 comprising a homozygous null mutation in Rag1 gene.

In exemplary embodiment 55, provided herein is a genetically modified rodent cell of embodiment 53 or 54, wherein the null mutation in the rodent Flt3 gene comprises an insertion, a deletion, and/or a substitution in the endogenous Flt3 gene.

In exemplary embodiment 56, provided herein is a genetically modified rodent cell of embodiment 55, wherein the null mutation in the rodent Flt3 gene is a deletion of the full Flt3 endogenous coding sequence.

In exemplary embodiment 57, provided herein is a genetically modified rodent cell of embodiment 56, wherein the genetically modified rodent cell is a mouse cell, and the mouse cell comprises a homozygous deletion of nucleic acid sequence between coordinates chr5: 147331171-147400265 (GRCm38 assembly).

In exemplary embodiment 58, provided herein is a genetically modified rodent cell of any one of embodiments 53-57, wherein the rodent portion of the Flt31 gene comprises exon 1, a non-coding portion of exon 2, and exons downstream of exon 6 of a rodent Flt31 gene.

In exemplary embodiment 59, provided herein is a genetically modified rodent cell of embodiment 58, wherein the exon 1, the non-coding portion of exon 2, and the exons downstream of exon 6 of the rodent Flt31 gene are at least 90%, at least 95%, or 100% identical to the corresponding exon 1, non-coding portion of exon 2, and exons downstream of exon 6 of a rodent Flt31 gene shown in Table 1A.

In exemplary embodiment 60, provided herein is a genetically modified rodent cell of any one of embodiments 1-59, wherein the human portion of the Flt31 gene comprises a signal peptide-coding portion of exon 2, and exons 3-6 of a human FLT3L gene.

In exemplary embodiment 61, provided herein is a genetically modified rodent cell of embodiment 60, wherein the signal peptide-coding portion of exon 2, and exons 3-6 of the human FLT3L gene are at least 50%, at least 60%, at least 70%, at least 80%, at least 90%, at least 95%, or 100% identical to the corresponding signal peptide-coding portion of exon 2, and exons 3-6 of a human FLT3L gene shown in Table 1B.

In exemplary embodiment 62, provided herein is a genetically modified rodent cell of any one of embodiments 53-61, wherein the Flt31 gene comprises rodent exon 1, rodent non-coding portion of exon 2, human signal peptide-coding portion of exon 2, human exons 3-6, and rodent exons 7-9.

In exemplary embodiment 63, provided herein is a genetically modified rodent cell of any one of embodiments 53-62, wherein the Flt31 gene encodes a chimeric membrane bound FLT3L comprising a signal peptide and cytokine-like core domain of a human FLT3L polypeptide, and a C-terminal portion of a rodent Flt31 polypeptide.

In exemplary embodiment 64, provided herein is a genetically modified rodent cell of embodiment 63, wherein the C-terminal portion of the rodent Flt31 polypeptide comprises a rodent stalk region, a rodent transmembrane domain, and a rodent cytoplasmic tail as shown in FIG. 1E.

In exemplary embodiment 65, provided herein is a genetically modified rodent cell of embodiment 63 or 64, wherein the C-terminal portion of the rodent Flt31 polypeptide has an amino acid sequence that is at least 70%, at least 80%, at least 90%, at least 95%, or 100% identical to a C-terminal portion of a rodent Flt31 polypeptide shown in Table 2.

In exemplary embodiment 66, provided herein is a genetically modified rodent cell of any one of embodiments 53-65, wherein the rodent cell expresses both soluble and membrane-bound forms of the Flt31 polypeptide.

In exemplary embodiment 67, provided herein is a genetically modified rodent cell of embodiment 66, wherein the soluble and membrane-bound forms of the Flt31 polypeptide comprise a signal peptide and a cytokine-like core domain of a human FLT3L polypeptide.

In exemplary embodiment 68, provided herein is a genetically modified rodent cell of any one of embodiments 63-67, wherein the signal peptide of the human FLT3L polypeptide comprises amino acids corresponding to residues 1-26 of a human FLT3L polypeptide.

In exemplary embodiment 69, provided herein is a genetically modified rodent cell of any one of embodiments 63-68, wherein the signal peptide of the human FLT3L polypeptide comprises an amino acid sequence that is at least 70%, at least 80%, at least 90%, at least 95%, or 100% identical to a corresponding amino acid sequence of a signal peptide of a human FLT3L polypeptide that appears in Table 2.

In exemplary embodiment 70, provided herein is a genetically modified rodent cell of any one of embodiments 63-69, wherein the cytokine-like core domain of the human FLT3L polypeptide comprises amino acids corresponding to residues 27-159 of a human FLT3L polypeptide.

In exemplary embodiment 71, provided herein is a genetically modified rodent cell of any one of embodiments 63-70, wherein the cytokine-like core domain of the human FLT3L polypeptide comprises an amino acid sequence that is at least 70%, at least 80%, at least 90%, at least 95%, or 100% identical to a corresponding amino acid sequence of cytokine-like core domains of a human FLT3L polypeptide that appears in Table 2.

In exemplary embodiment 72, provided herein is a genetically modified rodent cell of any one of embodiments 53-71, wherein the genetically modified rodent cell is heterozygous for the Flt3 ligand (Flt31) gene that comprises a rodent portion and a human portion.

In exemplary embodiment 73, provided herein is a genetically modified rodent cell of any one of embodiments 53-71, wherein the genetically modified rodent cell is homozygous for the Flt3 ligand (Flt31) gene that comprises a rodent portion and a human portion.

In exemplary embodiment 74, provided herein is a genetically modified rodent cell of any one of embodiments 53-73, wherein the rodent portion of the Flt31 gene is an endogenous rodent portion of the Flt31 gene and the rodent Flt31 gene is an endogenous rodent gene.

In exemplary embodiment 75, provided herein is a genetically modified rodent cell of any one of embodiments 53-74, wherein the rodent Flt31 polypeptide is an endogenous rodent Flt31 polypeptide.

In exemplary embodiment 76, provided herein is a genetically modified rodent cell of any one of embodiments 53-75, wherein the Flt31 promoter is a rodent promoter.

In exemplary embodiment 77, provided herein is a genetically modified rodent cell of embodiment 75 or 76, wherein the Flt31 promoter is an endogenous rodent promoter.

In exemplary embodiment 78, provided herein is a genetically modified rodent cell of any one of embodiments 75-77, wherein the Flt31 promoter is at the endogenous rodent gene locus.

In exemplary embodiment 79, provided herein is a genetically modified rodent cell of any one of embodiments 53-78, wherein the genetically modified rodent cell further comprises a nucleic acid that encodes a human or humanized SIRPA polypeptide, and wherein the nucleic acid is operably linked to a Sirpa promoter.

In exemplary embodiment 80, provided herein is a genetically modified rodent cell of embodiment 79, wherein the genetically modified rodent cell further comprises a Sirpa gene that encodes a Sirpa polypeptide comprising an extracellular portion of a human SIRPA polypeptide and an intracellular portion of a rodent Sirpa polypeptide, wherein the Sirpa gene is operably linked to a Sirpa promoter.

In exemplary embodiment 81, provided herein is a genetically modified rodent cell of embodiment 80, wherein the Sirpa gene comprises exons 1, 5, 6, 7 and 8 of a rodent Sirpa gene and exons 2-4 of a human SIRPA gene.

In exemplary embodiment 82, provided herein is a genetically modified rodent cell of embodiment 80 or 81, wherein the genetically modified rodent cell expresses a Sirpa polypeptide comprising an extracellular portion of a human SIRPA polypeptide and an intracellular portion of a rodent Sirpa polypeptide.

In exemplary embodiment 83, provided herein is a genetically modified rodent cell of any one of embodiments 80-82, wherein the rodent Sirpa polypeptide is an endogenous rodent Sirpa polypeptide, and/or the rodent Sirpa gene is an endogenous rodent gene.

In exemplary embodiment 84, provided herein is a genetically modified rodent cell of embodiment 79, wherein the genetically modified rodent cell expresses a human SIRPA polypeptide.

In exemplary embodiment 85, provided herein is a genetically modified rodent cell of any one of embodiments 79-84, wherein the genetically modified rodent cell further comprises: (1) a nucleic acid that encodes a human GM-CSF protein operably linked to a GM-CSF promoter; and/or (2) a nucleic acid that encodes a human IL3 protein operably linked to a IL3 promoter.

In exemplary embodiment 86, provided herein is a genetically modified rodent cell of any one of embodiments 79-85, wherein the SIRPα promoter, the GM-CSF promoter, and/or the IL3 promoter is a rodent promoter.

In exemplary embodiment 87, provided herein is a genetically modified rodent cell of embodiment 86, wherein the SIRPα promoter, the GM-CSF promoter, and/or the IL3 promoter is an endogenous rodent promoter.

In exemplary embodiment 88, provided herein is a genetically modified rodent cell of embodiment 86 or 87, wherein the SIRPα promoter, the GM-CSF promoter, and/or the IL3 promoter is at the corresponding endogenous rodent gene locus.

In exemplary embodiment 89, provided herein is a genetically modified rodent cell of any one of embodiments 79-88, comprising a null mutation in at least one corresponding rodent gene at the corresponding rodent gene locus.

In exemplary embodiment 90, provided herein is a genetically modified rodent cell of any one of embodiments 79-89, wherein the genetically modified rodent cell is heterozygous for at least one allele comprising the nucleic acid sequence that encodes the human or humanized protein.

In exemplary embodiment 91, provided herein is a genetically modified rodent cell of any one of embodiments 79-90, wherein the genetically modified rodent cell is homozygous for at least one allele comprising the nucleic acid sequence that encodes the human or humanized protein.

In exemplary embodiment 92, provided herein is a genetically modified rodent cell of any one of embodiments 53-91, wherein the rodent cell is a rat cell or a mouse cell.

In exemplary embodiment 93, provided herein is a genetically modified rodent cell of embodiment 92, wherein the rodent cell is a mouse cell.

In exemplary embodiment 94, provided herein is a genetically modified rodent cell of any one of embodiments 53-65, 68-81, 83, and 85-93, wherein the genetically modified rodent cell is a rodent embryonic stem (ES) cell.

In exemplary embodiment 95, provided herein is a genetically modified rodent embryonic stem cell, comprising in its genome: (i) a homozygous null mutation in Rag2 gene; (ii) a homozygous null mutation in IL2rg gene; (iii) a homozygous null mutation in the rodent FMS-like tyrosine kinase 3 (Flt3) gene; and (iv) a Flt3 ligand (Flt31) gene that comprises a rodent portion and a human portion operably linked to a Flt31 promoter.

In exemplary embodiment 96, provided herein is a genetically modified rodent embryonic stem cell of embodiment 95 comprising a homozygous null mutation in Rag1 gene.

In exemplary embodiment 97, provided herein is a genetically modified rodent embryonic stem cell of embodiment 95 or 96, wherein the null mutation in the rodent Flt3 gene comprises an insertion, a deletion, and/or a substitution in the endogenous Flt3 gene.

In exemplary embodiment 98, provided herein is a genetically modified rodent embryonic stem cell of embodiment 97, wherein the null mutation in the rodent Flt3 gene is a deletion of the full Flt3 endogenous coding sequence.

In exemplary embodiment 99, provided herein is a genetically modified rodent embryonic stem cell of embodiment 98, wherein the genetically modified rodent embryonic stem cell is a mouse embryonic stem cell, and the mouse embryonic stem cell comprises a homozygous deletion of nucleic acid sequence between coordinates chr5: 147331171-147400265 (GRCm38 assembly).

In exemplary embodiment 100, provided herein is a genetically modified rodent embryonic stem cell of any one of embodiments 95-99, wherein the rodent portion of the Flt31 gene comprises exon 1, a non-coding portion of exon 2, and exons downstream of exon 6 of a rodent Flt31 gene.

In exemplary embodiment 101, provided herein is a genetically modified rodent embryonic stem cell of embodiment 100, wherein the exon 1, the non-coding portion of exon 2, and the exons downstream of exon 6 of the rodent Flt31 gene are at least 90%, at least 95%, or 100% identical to the corresponding exon 1, non-coding portion of exon 2, and exons downstream of exon 6 of a rodent Flt31 gene shown in Table 1A.

In exemplary embodiment 102, provided herein is a genetically modified rodent embryonic stem cell of any one of embodiments 95-101, wherein the human portion of the Flt31 gene comprises a signal peptide-coding portion of exon 2, and exons 3-6 of a human FLT3L gene.

In exemplary embodiment 103, provided herein is a genetically modified rodent embryonic stem cell of embodiment 102, wherein the signal peptide-coding portion of exon 2, and exons 3-6 of the human FLT3L gene are at least 50%, at least 60%, at least 70%, at least 80%, at least 90%, at least 95%, or 100% identical to the corresponding signal peptide-coding portion of exon 2, and exons 3-6 of a human FLT3L gene shown in Table 1B.

In exemplary embodiment 104, provided herein is a genetically modified rodent embryonic stem cell of any one of embodiments 95-103, wherein the Flt31 gene comprises rodent exon 1, rodent non-coding portion of exon 2, human signal peptide-coding portion of exon 2, human exons 3-6, and rodent exons 7-9.

In exemplary embodiment 105, provided herein is a genetically modified rodent embryonic stem cell of any one of embodiments 95-104, wherein the Flt31 gene encodes a chimeric membrane bound FLT3L comprising a signal peptide and cytokine-like core domain of a human FLT3L polypeptide, and a C-terminal portion of a rodent Flt31 polypeptide.

In exemplary embodiment 106, provided herein is a genetically modified rodent embryonic stem cell of embodiment 105, wherein the C-terminal portion of the rodent Flt31 polypeptide comprises a rodent stalk region, a rodent transmembrane domain, and a rodent cytoplasmic tail as shown in FIG. 1E.

In exemplary embodiment 107, provided herein is a genetically modified rodent embryonic stem cell of embodiment 105 or 106, wherein the C-terminal portion of the rodent Flt31 polypeptide has an amino acid sequence that is at least 70%, at least 80%, at least 90%, at least 95%, or 100% identical to a C-terminal portion of a rodent Flt31 polypeptide shown in Table 2.

In exemplary embodiment 108, provided herein is a genetically modified rodent embryonic stem cell of any one of embodiments 105-107, wherein the signal peptide of the human FLT3L polypeptide comprises amino acids corresponding to residues 1-26 of a human FLT3L polypeptide.

In exemplary embodiment 109, provided herein is a genetically modified rodent embryonic stem cell of any one of embodiments 105-108, wherein the signal peptide of the human FLT3L polypeptide comprises an amino acid sequence that is at least 70%, at least 80%, at least 90%, at least 95%, or 100% identical to a corresponding amino acid sequence of a signal peptide of a human FLT3L polypeptide that appears in Table 2.

In exemplary embodiment 110, provided herein is a genetically modified rodent embryonic stem cell of any one of embodiments 105-109, wherein the cytokine-like core domain of the human FLT3L polypeptide comprises amino acids corresponding to residues 27-159 of a human FLT3L polypeptide.

In exemplary embodiment 111, provided herein is a genetically modified rodent embryonic stem cell of any one of embodiments 105-110, wherein the cytokine-like core domain of the human FLT3L polypeptide comprises an amino acid sequence that is at least 70%, at least 80%, at least 90%, at least 95%, or 100% identical to a corresponding amino acid sequence of cytokine-like core domains of a human FLT3L polypeptide that appears in Table 2.

In exemplary embodiment 112, provided herein is a genetically modified rodent embryonic stem cell of any one of embodiments 95-111, wherein the genetically modified rodent embryonic stem cell is heterozygous for the Flt3 ligand (Flt31) gene that comprises a rodent portion and a human portion.

In exemplary embodiment 113, provided herein is a genetically modified rodent embryonic stem cell of any one of embodiments 95-111, wherein the genetically modified rodent embryonic stem cell is homozygous for the Flt3 ligand (Flt31) gene that comprises a rodent portion and a human portion.

In exemplary embodiment 114, provided herein is a genetically modified rodent embryonic stem cell of any one of embodiments 95-113, wherein the rodent portion of the Flt31 gene is an endogenous rodent portion of the Flt31 gene and the rodent Flt31 gene is an endogenous rodent gene.

In exemplary embodiment 115, provided herein is a genetically modified rodent embryonic stem cell of any one of embodiments 95-114, wherein the rodent Flt31 polypeptide is an endogenous rodent Flt31 polypeptide.

In exemplary embodiment 116, provided herein is a genetically modified rodent embryonic stem cell of any one of embodiments 95-115, wherein the Flt31 promoter is a rodent promoter.

In exemplary embodiment 117, provided herein is a genetically modified rodent embryonic stem cell of embodiment 115 or 116, wherein the Flt31 promoter is an endogenous rodent promoter.

In exemplary embodiment 118, provided herein is a genetically modified rodent embryonic stem cell of any one of embodiments 115-117, wherein the Flt31 promoter is at the endogenous rodent gene locus.

In exemplary embodiment 119a, provided herein is a genetically modified rodent embryonic stem cell of any one of claims 95-118, wherein the genetically modified rodent embryonic stem cell further comprises a nucleic acid that encodes a human or humanized SIRPA polypeptide, and wherein the nucleic acid is operably linked to a Sirpa promoter.

In exemplary embodiment 119b, provided herein is a genetically modified rodent embryonic stem cell of embodiment 119a, wherein the genetically modified rodent embryonic stem cell further comprises a Sirpa gene that encodes a Sirpa polypeptide comprising an extracellular portion of a human SIRPA polypeptide and an intracellular portion of a rodent Sirpa polypeptide, wherein the Sirpa gene is operably linked to a Sirpa promoter.

In exemplary embodiment 120, provided herein is a genetically modified rodent embryonic stem cell of embodiment 119b, wherein the Sirpa gene comprises exons 1, 5, 6, 7 and 8 of a rodent Sirpa gene and exons 2-4 of a human SIRPA gene.

In exemplary embodiment 121a, provided herein is a genetically modified rodent embryonic stem cell of embodiment 119b or 120, wherein the rodent Sirpa polypeptide is an endogenous rodent Sirpa polypeptide, and/or the rodent Sirpa gene is an endogenous rodent gene.

In exemplary embodiment 121b, provided herein is a genetically modified rodent embryonic stem cell of any one of embodiments 119a-121a, wherein the genetically modified rodent embryonic stem cell further comprises (1) a nucleic acid that encodes a human GM-CSF protein operably linked to a GM-CSF promoter; and/or (2) a nucleic acid that encodes a human IL3 protein operably linked to a IL3 promoter.

In exemplary embodiment 122, provided herein is a genetically modified rodent embryonic stem cell of any one of embodiments 119a-121b, wherein the SIRPα promoter, the GM-CSF promoter, and/or the IL3 promoter is a rodent promoter.

In exemplary embodiment 123, provided herein is a genetically modified rodent embryonic stem cell of embodiment 122, wherein the SIRPα promoter, the GM-CSF promoter, and/or the IL3 promoter is an endogenous rodent promoter.

In exemplary embodiment 124, provided herein is a genetically modified rodent embryonic stem cell of embodiment 122 or 123, wherein the SIRPα promoter, the GM-CSF promoter, and/or the IL3 promoter is at the corresponding endogenous rodent gene locus.

In exemplary embodiment 125, provided herein is a genetically modified rodent embryonic stem cell of any one of embodiments 119a-124, comprising a null mutation in at least one corresponding rodent gene at the corresponding rodent gene locus.

In exemplary embodiment 126, provided herein is a genetically modified rodent embryonic stem cell of any one of embodiments 119a-125, wherein the genetically modified rodent embryonic stem cell is heterozygous for at least one allele comprising the nucleic acid sequence that encodes the human or humanized protein.

In exemplary embodiment 127, provided herein is a genetically modified rodent embryonic stem cell of any one of embodiments 119a-126, wherein the genetically modified rodent embryonic stem cell is homozygous for at least one allele comprising the nucleic acid sequence that encodes the human or humanized protein.

In exemplary embodiment 128, provided herein is a genetically modified rodent embryonic stem cell of any one of embodiments 95-127, wherein the rodent embryonic stem cell is a rat embryonic stem cell or a mouse embryonic stem cell.

In exemplary embodiment 129, provided herein is a genetically modified rodent embryonic stem cell of embodiment 128, wherein the rodent embryonic stem cell is a mouse embryonic stem cell.

In exemplary embodiment 130, provided herein is a method of making a rodent embryonic stem cell, comprising genetically engineering the rodent embryonic stem cell so that the rodent embryonic stem cell has a genome that comprises: (i) a homozygous null mutation in Rag2 gene; (ii) a homozygous null mutation in IL2rg gene; (iii) a homozygous null mutation in the rodent FMS-like tyrosine kinase 3 (Flt3) gene; and (iv) a Flt3 ligand (Flt31) gene that comprises a rodent portion and a human portion operably linked to a Flt31 promoter.

In exemplary embodiment 131, provided herein is a method of embodiment 130, wherein the rodent embryonic stem cell is further engineered to have a genome that comprises a homozygous null mutation in Rag1 gene.

In exemplary embodiment 132, provided herein is a method of embodiment 130 or 131, wherein the null mutation in the rodent Flt3 gene comprises an insertion, a deletion, and/or a substitution in the endogenous Flt3 gene.

In exemplary embodiment 133, provided herein is a method of embodiment 132, wherein the null mutation in the rodent Flt3 gene is a deletion of the full Flt3 endogenous coding sequence.

In exemplary embodiment 134, provided herein is a method of embodiment 133, wherein the genetically modified rodent embryonic stem cell is a mouse embryonic stem cell, and the mouse embryonic stem cell comprises a homozygous deletion of nucleic acid sequence between coordinates chr5: 147331171-147400265 (GRCm38 assembly).

In exemplary embodiment 135, provided herein is a method of any one of embodiments 130-134, wherein the rodent portion of the Flt31 gene comprises exon 1, a non-coding portion of exon 2, and exons downstream of exon 6 of a rodent Flt31 gene.

In exemplary embodiment 136, provided herein is a method of embodiment 135, wherein the exon 1, the non-coding portion of exon 2, and the exons downstream of exon 6 of the rodent Flt31 gene are at least 90%, at least 95%, or 100% identical to the corresponding exon 1, non-coding portion of exon 2, and exons downstream of exon 6 of a rodent Flt31 gene shown in Table 1A.

In exemplary embodiment 137, provided herein is a method of any one of embodiments 130-136, wherein the human portion of the Flt31 gene comprises a signal peptide-coding portion of exon 2, and exons 3-6 of a human FLT3L gene.

In exemplary embodiment 138, provided herein is a method of embodiment 137, wherein the signal peptide-coding portion of exon 2, and exons 3-6 of the human FLT3L gene are at least 50%, at least 60%, at least 70%, at least 80%, at least 90%, at least 95%, or 100% identical to the corresponding signal peptide-coding portion of exon 2, and exons 3-6 of a human FLT3L gene shown in Table 1B.

In exemplary embodiment 139, provided herein is a method of any one of embodiments 130-138, wherein the Flt31 gene comprises rodent exon 1, rodent non-coding portion of exon 2, human signal peptide-coding portion of exon 2, human exons 3-6, and rodent exons 7-9.

In exemplary embodiment 140, provided herein is a method of any one of embodiments 130-139, wherein the Flt31 gene encodes a chimeric membrane bound FLT3L comprising a signal peptide and cytokine-like core domain of a human FLT3L polypeptide, and a C-terminal portion of a rodent Flt31 polypeptide.

In exemplary embodiment 141, provided herein is a method of embodiment 140, wherein the C-terminal portion of the rodent Flt31 polypeptide comprises a rodent stalk region, a rodent transmembrane domain, and a rodent cytoplasmic tail as shown in FIG. 1E.

In exemplary embodiment 142, provided herein is a method of embodiment 140 or 141, wherein the C-terminal portion of the rodent Flt31 polypeptide has an amino acid sequence that is at least 70%, at least 80%, at least 90%, at least 95%, or 100% identical to a C-terminal portion of a rodent Flt31 polypeptide shown in Table 2.

In exemplary embodiment 143, provided herein is a method of any one of embodiments 140-142, wherein the signal peptide of the human FLT3L polypeptide comprises amino acids corresponding to residues 1-26 of a human FLT3L polypeptide.

In exemplary embodiment 144, provided herein is a method of any one of embodiments 140-143, wherein the signal peptide of the human FLT3L polypeptide comprises an amino acid sequence that is at least 70%, at least 80%, at least 90%, at least 95%, or 100% identical to a corresponding amino acid sequence of a signal peptide of a human FLT3L polypeptide that appears in Table 2.

In exemplary embodiment 145, provided herein is a method of any one of embodiments 140-144, wherein the cytokine-like core domain of the human FLT3L polypeptide comprises amino acids corresponding to residues 27-159 of a human FLT3L polypeptide.

In exemplary embodiment 146, provided herein is a method of any one of embodiments 140-145, wherein the cytokine-like core domain of the human FLT3L polypeptide comprises an amino acid sequence that is at least 70%, at least 80%, at least 90%, at least 95%, or 100% identical to a corresponding amino acid sequence of cytokine-like core domains of a human FLT3L polypeptide that appears in Table 2.

In exemplary embodiment 147, provided herein is a method of any one of embodiments 130-146, wherein the genetically modified rodent embryonic stem cell is heterozygous for the Flt3 ligand (Flt31) gene that comprises a rodent portion and a human portion.

In exemplary embodiment 148, provided herein is a method of any one of embodiments 130-146, wherein the genetically modified rodent embryonic stem cell is homozygous for the Flt3 ligand (Flt31) gene that comprises a rodent portion and a human portion.

In exemplary embodiment 149, provided herein is a method of any one of embodiments 130-148, wherein the rodent portion of the Flt31 gene is an endogenous rodent portion of the Flt31 gene and the rodent Flt31 gene is an endogenous rodent gene.

In exemplary embodiment 150, provided herein is a method of any one of embodiments 130-149, wherein the rodent Flt31 polypeptide is an endogenous rodent Flt31 polypeptide.

In exemplary embodiment 151, provided herein is a method of any one of embodiments 130-150, wherein the Flt31 promoter is a rodent promoter.

In exemplary embodiment 152, provided herein is a method of embodiment 150 or 151, wherein the Flt31 promoter is an endogenous rodent promoter.

In exemplary embodiment 153, provided herein is a method of any one of embodiments 150-152, wherein the Flt31 promoter is at the endogenous rodent gene locus.

In exemplary embodiment 154, provided herein is a method of embodiment 153, wherein the genetically modified rodent embryonic stem cell is further engineered to comprise in its genome a Sirpa gene that encodes a Sirpa polypeptide comprising an extracellular portion of a human SIRPA polypeptide and an intracellular portion of a rodent Sirpa polypeptide, wherein the Sirpa gene is operably linked to a Sirpa promoter.

In exemplary embodiment 155, provided herein is a method of embodiment 154, wherein the Sirpa gene comprises exons 1, 5, 6, 7 and 8 of a rodent Sirpa gene and exons 2-4 of a human SIRPA gene.

In exemplary embodiment 156a, provided herein is a method of embodiment 154 or 155, wherein the rodent Sirpa polypeptide is an endogenous rodent Sirpa polypeptide, and/or the rodent Sirpa gene is an endogenous rodent gene.

In exemplary embodiment 156b, provided herein is a method of any one of embodiments 154-156a, wherein the genetically modified rodent embryonic stem cell is further engineered to comprise in its genome a nucleic acid encoding a human GM-CSF protein and operably linked to a GM-CSF promoter, and/or a nucleic acid encoding a human IL3 protein and operably linked to a IL3 promoter.

In exemplary embodiment 157, provided herein is a method of any one of embodiments 154-156b, wherein the SIRPα promoter, the GM-CSF promoter, and/or the IL3 promoter is a rodent promoter.

In exemplary embodiment 158, provided herein is a method of embodiment 157, wherein the SIRPα promoter, the GM-CSF promoter, and/or the IL3 promoter is an endogenous rodent promoter.

In exemplary embodiment 159, provided herein is a method of embodiment 156 or 157, wherein the SIRPα promoter, the GM-CSF promoter, and/or the IL3 promoter is at the corresponding endogenous rodent gene locus.

In exemplary embodiment 160, provided herein is a method of any one of embodiments 154-159, comprising a null mutation in at least one corresponding rodent gene at the corresponding rodent gene locus.

In exemplary embodiment 161, provided herein is a method of any one of embodiments 154-160, wherein the genetically modified rodent embryonic stem cell is heterozygous for at least one allele comprising the nucleic acid sequence that encodes the human or humanized protein.

In exemplary embodiment 162, provided herein is a method of any one of embodiments 154-161, wherein the genetically modified rodent embryonic stem cell is homozygous for at least one allele comprising the nucleic acid sequence that encodes the human or humanized protein.

In exemplary embodiment 163, provided herein is a method of any one of embodiments 130-162, wherein the rodent embryonic stem cell is a rat embryonic stem cell or a mouse embryonic stem cell.

In exemplary embodiment 164, provided herein is a method of embodiment 163, wherein the rodent embryonic stem cell is a mouse embryonic stem cell.

In exemplary embodiment 165, provided herein is a rodent embryo comprising the rodent embryonic stem cell of any one of embodiments 94-129, or the rodent embryonic stem cell made according to the method of any one of embodiments 130-164.

In exemplary embodiment 166, provided herein is a method of making a rodent comprising in its genome: (i) a homozygous null mutation in Rag2 gene; (ii) a homozygous null mutation in IL2rg gene; (iii) a homozygous null mutation in the rodent FMS-like tyrosine kinase 3 (Flt3) gene; and (iv) a Flt3 ligand (Flt31) gene that comprises a rodent portion and a human portion operably linked to a Flt31 promoter, the method comprising steps of: (a) obtaining a rodent embryonic stem cell of any one of embodiments 94-129, or the rodent embryonic stem cell made according to the method of any one of embodiments 130-164; and (b) creating a rodent using the rodent embryonic cell of (a).

In exemplary embodiment 167, provided herein is a method of making a rodent comprising in its genome: (i) a homozygous null mutation in Rag2 gene; (ii) a homozygous null mutation in IL2rg gene; (iii) a homozygous null mutation in the rodent FMS-like tyrosine kinase 3 (Flt3) gene; and (iv) a Flt3 ligand (Flt31) gene that comprises a rodent portion and a human portion operably linked to a Flt31 promoter, the method comprising modifying the genome of the rodent so that it comprises: (i) a homozygous null mutation in Rag2 gene; (ii) a homozygous null mutation in IL2rg gene; (iii) a homozygous null mutation in the rodent FMS-like tyrosine kinase 3 (Flt3) gene; and (iv) a Flt3 ligand (Flt31) gene that comprises a rodent portion and a human portion operably linked to a Flt31 promoter.

In exemplary embodiment 168, provided herein is a method of embodiment 167, wherein the rodent is engineered to comprise a homozygous null mutation in Rag1 gene.

In exemplary embodiment 169, provided herein is a method of embodiment 167 or 168, wherein the null mutation in the rodent Flt3 gene comprises an insertion, a deletion, and/or a substitution in the endogenous Flt3 gene.

In exemplary embodiment 170, provided herein is a method of embodiment 169, wherein the null mutation in the rodent Flt3 gene is a deletion of the full Flt3 endogenous coding sequence.

In exemplary embodiment 171, provided herein is a method of embodiment 170, wherein the genetically modified rodent is a mouse, and the mouse comprises a homozygous deletion of nucleic acid sequence between coordinates chr5: 147331171-147400265 (GRCm38 assembly).

In exemplary embodiment 172, provided herein is a method of any one of embodiments 167-171, wherein the rodent portion of the Flt31 gene comprises exon 1, a non-coding portion of exon 2, and exons downstream of exon 6 of a rodent Flt31 gene.

In exemplary embodiment 173, provided herein is a method of embodiment 172, wherein the exon 1, the non-coding portion of exon 2, and the exons downstream of exon 6 of the rodent Flt31 gene are at least 90%, at least 95%, or 100% identical to the corresponding exon 1, non-coding portion of exon 2, and exons downstream of exon 6 of a rodent Flt31 gene shown in Table 1A.

In exemplary embodiment 174, provided herein is a method of any one of embodiments 167-173, wherein the human portion of the Flt31 gene comprises a signal peptide-coding portion of exon 2, and exons 3-6 of a human FLT3L gene.

In exemplary embodiment 175, provided herein is a method of embodiment 174, wherein the signal peptide-coding portion of exon 2, and exons 3-6 of the human FLT3L gene are at least 50%, at least 60%, at least 70%, at least 80%, at least 90%, at least 95%, or 100% identical to the corresponding signal peptide-coding portion of exon 2, and exons 3-6 of a human FLT3L gene shown in Table 1B.

In exemplary embodiment 176, provided herein is a method of any one of embodiments 167-175, wherein the Flt31 gene comprises rodent exon 1, rodent non-coding portion of exon 2, human signal peptide-coding portion of exon 2, human exons 3-6, and rodent exons 7-9.

In exemplary embodiment 177, provided herein is a method of any one of embodiments 167-176, wherein the Flt31 gene encodes a chimeric membrane bound FLT3L comprising a signal peptide and cytokine-like core domain of a human FLT3L polypeptide, and a C-terminal portion of a rodent Flt31 polypeptide.

In exemplary embodiment 178, provided herein is a method of embodiment 177, wherein the C-terminal portion of the rodent Flt31 polypeptide comprises a rodent stalk region, a rodent transmembrane domain, and a rodent cytoplasmic tail as shown in FIG. 1E.

In exemplary embodiment 179, provided herein is a method of embodiment 177 or 178, wherein the C-terminal portion of the rodent Flt31 polypeptide has an amino acid sequence that is at least 70%, at least 80%, at least 90%, at least 95%, or 100% identical to a C-terminal portion of a rodent Flt31 polypeptide shown in Table 2.

In exemplary embodiment 180, provided herein is a method of any one of embodiments 167-179, wherein the rodent expresses both soluble and membrane-bound forms of the Flt31 polypeptide.

In exemplary embodiment 181, provided herein is a method of embodiment 180, wherein the soluble and membrane-bound forms of the Flt31 polypeptide comprise a signal peptide and a cytokine-like core domain of a human FLT3L polypeptide.

In exemplary embodiment 182, provided herein is a method of any one of embodiments 177-181, wherein the signal peptide of the human FLT3L polypeptide comprises amino acids corresponding to residues 1-26 of a human FLT3L polypeptide.

In exemplary embodiment 183, provided herein is a method of any one of embodiments 177-182, wherein the signal peptide of the human FLT3L polypeptide comprises an amino acid sequence that is at least 70%, at least 80%, at least 90%, at least 95%, or 100% identical to a corresponding amino acid sequence of a signal peptide of a human FLT3L polypeptide that appears in Table 2.

In exemplary embodiment 184, provided herein is a method of any one of embodiments 177-183, wherein the cytokine-like core domain of the human FLT3L polypeptide comprises amino acids corresponding to residues 27-159 of a human FLT3L polypeptide.

In exemplary embodiment 185, provided herein is a method of any one of embodiments 177-184, wherein the cytokine-like core domain of the human FLT3L polypeptide comprises an amino acid sequence that is at least 70%, at least 80%, at least 90%, at least 95%, or 100% identical to a corresponding amino acid sequence of cytokine-like core domains of a human FLT3L polypeptide that appears in Table 2.

In exemplary embodiment 186, provided herein is a method of any one of embodiments 167-185, wherein the genetically modified rodent is heterozygous for the Flt3 ligand (Flt31) gene that comprises a rodent portion and a human portion.

In exemplary embodiment 187, provided herein is a method of any one of embodiments 167-185, wherein the genetically modified rodent is homozygous for the Flt3 ligand (Flt31) gene that comprises a rodent portion and a human portion.

In exemplary embodiment 188, provided herein is a method of any one of embodiments 167-187, wherein the rodent portion of the Flt31 gene is an endogenous rodent portion of the Flt31 gene and the rodent Flt31 gene is an endogenous rodent gene.

In exemplary embodiment 189, provided herein is a method of any one of embodiments 167-188, wherein the rodent Flt31 polypeptide is an endogenous rodent Flt31 polypeptide.

In exemplary embodiment 190, provided herein is a method of any one of embodiments 167-189, wherein the Flt31 promoter is a rodent promoter.

In exemplary embodiment 191, provided herein is a method of embodiment 189 or 190, wherein the Flt31 promoter is an endogenous rodent promoter.

In exemplary embodiment 192, provided herein is a method of any one of embodiments 189-191, wherein the Flt31 promoter is at the endogenous rodent gene locus.

In exemplary embodiment 193, provided herein is a method of any one of embodiments 167-192, wherein the genetically modified rodent expresses a human or humanized SIRPA polypeptide encoded by a nucleic acid operably linked to a Sirpa promoter.

In exemplary embodiment 194, provided herein is a method of embodiment 193, wherein the genetically modified rodent further comprises a Sirpa gene that encodes a Sirpa polypeptide comprising an extracellular portion of a human SIRPA polypeptide and an intracellular portion of a rodent Sirpa polypeptide, wherein the Sirpa gene is operably linked to a Sirpa promoter.

In exemplary embodiment 195, provided herein is a method of embodiment 194, wherein the Sirpa gene comprises exons 1, 5, 6, 7 and 8 of a rodent Sirpa gene and exons 2-4 of a human SIRPA gene.

In exemplary embodiment 196, provided herein is a method of embodiment 194 or 195, wherein the genetically modified rodent expresses a Sirpa polypeptide comprising an extracellular portion of a human SIRPA polypeptide and an intracellular portion of a rodent Sirpa polypeptide.

In exemplary embodiment 197, provided herein is a method of any one of embodiments 194-196, wherein the rodent Sirpa polypeptide is an endogenous rodent Sirpa polypeptide, and/or the rodent Sirpa gene is an endogenous rodent gene.

In exemplary embodiment 198, provided herein is a method of embodiment 193, wherein the genetically modified rodent expresses a human SIRPA polypeptide encoded by a nucleic acid operably linked to a Sirpa promoter.

In exemplary embodiment 199, provided herein is a method of any one of embodiments 193-198, wherein the genetically modified rodent further expresses a human GM-CSF protein encoded by a nucleic acid operably linked to a GM-CSF promoter and/or a human IL3 protein encoded by a nucleic acid operably linked to a IL3 promoter.

In exemplary embodiment 200, provided herein is a method of any one of embodiments 193-199, wherein the SIRPα promoter, the GM-CSF promoter, and/or the IL3 promoter is a rodent promoter.

In exemplary embodiment 201, provided herein is a method of embodiment 200, wherein the SIRPα promoter, the GM-CSF promoter, and/or the IL3 promoter is an endogenous rodent promoter.

In exemplary embodiment 202, provided herein is a method of embodiment 200 or 201, wherein the SIRPα promoter, the GM-CSF promoter, and/or the IL3 promoter is at the corresponding endogenous rodent gene locus.

In exemplary embodiment 203, provided herein is a method of any one of embodiments 193-202, wherein the genetically modified rodent comprises a null mutation in at least one corresponding rodent gene at the corresponding rodent gene locus.

In exemplary embodiment 204, provided herein is a method of any one of embodiments 193-203, wherein the genetically modified rodent is heterozygous for at least one allele comprising the nucleic acid sequence that encodes the human or humanized protein.

In exemplary embodiment 205, provided herein is a method of any one of embodiments 193-204, wherein the genetically modified rodent is homozygous for at least one allele comprising the nucleic acid sequence that encodes the human or humanized protein.

In exemplary embodiment 206, provided herein is a method of any one of embodiments 167-205, wherein the rodent is a mouse or a rat.

In exemplary embodiment 207, provided herein is a method of embodiment 206, wherein the rodent is a mouse.

### Examples

The following Examples and the accompanying Drawings are provided so as to describe to those of ordinary skill in the art how to make and use methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. The Examples do not include detailed descriptions of conventional methods that would be well known to those of ordinary skill in the art (molecular cloning techniques, etc.). Unless indicated otherwise, parts are parts by weight, molecular weight is average molecular weight, temperature is indicated in Celsius, and pressure is at or near atmospheric.

### Example 1: Generation of Genetically Engineered Mice

### Example 1.1: Generation of Humanized Flt3l Mouse

Mouse and human Flt3 ligands exist as multiple isoforms, both membrane bound and soluble forms, which are believed to arise from alternative splicing (Lyman et al. (1995) Oncogene 10:149-157; McClanahan et al. (1996) Blood 88(9):3371-3382; Lyman et al. (1995) Oncogene 11:1165-1172; Lyman and Jackbsen (1998) Blood 91(4) 1101-1134). The soluble isoform can also arise from proteolytic cleavage (resulting from cleavage by TNFalpha converting enzyme or TACE) (Kazi et al. (2019) Physiol. Rev. 99:1433-1466). All known soluble isoforms of human and mouse Flt3 ligand comprise a receptor binding domain (cytokine-like core domain), generally encoded by exons 3-6 of the gene. The mouse Flt3 ligand (Flt31) locus was humanized by using VELOCIGENE® technology (see, e.g., U.S. Pat. No. 6,586,251 and Valenzuela et al. (2003) High-throughput engineering of the mouse genome couple with high-resolution expression analysis. Nat. Biotech. 21(6): 652-659, both incorporated herein by reference). The resulting humanized Flt31 locus included the endogenous mouse Flt31 promoter, chimeric exon 2 comprising a mouse non-coding portion of exon 2 and a human signal peptide-coding portion of exon 2 (encoding human signal peptide), and human FLT3L exons 3 through 6 (encoding human FLT3L cytokine-like core domains), including 111 bp of human intron 6 sequence, followed by remaining endogenous mouse Flt31, which includes a remainder of intron 6 and exons 7-9, as well as the 3′UTR.

To create a final targeting vector for humanizing FLT3L: (i) part of mouse intron 2 through mouse exon 6 into intron 6 (2293 bp; GRCm38/mm110-chr7:45,133,715-45,136,007, which encode part of the mouse Flt31 signal sequence and cytokine-like core domain sequences, was first deleted in bacterial artificial chromosome (BAC) clone bMQ432L09 (Thermo-Fisher/Invitrogen), (ii) followed by introduction of part of human exon 2, at the ATG, through human exon 6 into intron 6 (4520 bp; GRCh38/hg38-chr19:49,474,640-49,479,159, which encode human FLT3L signal sequence and cytokine-like core domain sequences), and a Neomycin (Neo) resistance self-deleting cassette (with CRE recombinase controlled by Protamine promoter).

In detail, part of mouse intron 2 through mouse exon 6 into intron 6 (2293 bp), were first deleted in bacterial artificial chromosome (BAC) clone bMQ432L09, resulting in modified BAC #1, see FIG. 1A. Through bacterial homologous recombination (BHR), a spectinomycin cassette ligated to Up and Down homology boxes (260 bp & 230 bp) was used to replace the Flt31 sequences. The homology boxes were made by PCR using the BAC as a template and are indicated in the Table 4 below:

TABLE 4

| | | | Coordinates |
|---|---|---|---|
| Homology | 5′ Primer | 3′ Primer | (GRCm38/ |
| Box | (SEQ ID NO) | (SEQ ID NO) | mm10) |
| 5′; | GGGTTCTTAGA | GGTATACGCA | chr7: |
| 260 bp | AGAGGAGATG | CATTTGGGCT | 45,136,007- |
| | (SEQ ID | (SEQ ID | 45,136,266 |
| | NO: 13) | NO: 14) | |
| 3′; | GTGTGAGTG | GCCTGCATAA | chr7: |
| 230 bp | TTTTACCTA | TGAGACCCTA | 45,133,485- |
| | CATGCC | TC | 45,133,714 |
| | (SEQ ID | (SEQ ID | |
| | NO: 15) | NO: 16) | |

To humanize the BAC #1 described above, an initial plasmid was generated to carry a nucleic acid encoding the human FLT3L signal peptide and cytokine-like core domains along with 5′ mouse Flt31 sequence. More specifically, this initial plasmid contained from 5′ to 3′: (i) a NotI site, (ii) a mouse Flt31 sequence of 382 bp (a 3′ portion of mouse intron 1 (348 bp) and a 5′ portion (i.e., non-coding portion) of mouse exon 2 (34 bp)), (iii) a human FLT3L nucleic acid sequence (from modified human BAC CTD2523c15 (ThermoFisher/Invitrogen)) which included a portion of exon 2 from the ATG (33 bp), intron 2 (1018 bp), exon 3 (111 bp), intron 3 (343 bp), exon 4 (54 bp), intron 4 (224 bp), exon 5 (144 bp), intron 5 (2343 bp), exon 6 (139 bp), and a portion of intron 6 (111 bp), and (v) SpeI site. This donor plasmid was digested with NotI and SpeI to release the fragment and subsequently ligated to, from 5′ to 3′: (i) a selection cassette containing a hygromycin resistance gene operably linked to a EM7 promoter and (ii) a mouse Flt31 sequence of 230 bp (a 3′ portion of mouse intron 6). This BHR donor fragment containing the human FLT3L nucleic acid sequence and the selection cassette, flanked by mouse Flt31 sequence (the Up-Box and the Down-Box) was used to replace the spectinomycin cassette through BHR, resulting in BAC #2 (see FIG. 1B, step A). The homology boxes were made by PCR using the BAC as a template and are indicated in the Table 5 below:

TABLE 5

| | | | Coordinates |
|---|---|---|---|
| Homology | 5′ Primer | 3′ Primer | (GRCm38/ |
| Box | (SEQ ID NO) | (SEQ ID NO) | mm10) |
| 5′; | TTGGCTGTCA | CTCTGCCGG | chr7: |
| 382 bp | TCCAGCTTT | GGACCCCTC | 45,136,051- |
| | G (SEQ ID | AT (SEQ ID | 45,136,432 |
| | NO: 17) | NO: 18) | |
| 3′; | GTGTGAGTG | AAGGAAAG | chr7: |
| 325 bp | TTTTACCTAC | GAAGCCAGA | 45,133,390- |
| | ATGCC | TGTTC | 45,133,714 |
| | (SEQ ID | (SEQ ID | |
| | NO: 19) | NO: 20) | |

To make the final targeting vector from the previously described BAC #2, a Neomycin (Neo) resistance self-deleting cassette (with CRE recombinase controlled by Protamine promoter) flanked by loxp sites (loxp-neo-loxp), replaced the EM7-Hyg cassette by BHR (see FIG. 1B, step B).

The final targeting vector (BAC #3) contained from 5′ to 3′: the 5′ mouse homology arm, human FLT3L signal peptide and cytokine-like core domains, the loxp-Neo-loxp self-deleting cassette, the 3′ mouse homology arm, and the chloramphenicol resistance cassette (CM; not depicted on FIG. 1B); the final clone was selected based on Neo/CM resistance.

The final targeting vector was electroporated into mouse embryonic stem (ES) cells comprising hSIRP-alpha (or human SIRPA), RAG2−/− IL2Rg−/− modifications (e.g., described in WO 2014/071397, U.S. Pat. No. 11,019,810, WO 2016/168212, each of which is incorporated by reference herein). Targeted homologous recombination resulted in replacement of 2336 bp kb of mouse sequence (GRCm38/mm10 coordinates chr7:45,133,715-45,136,050) with 4520 bp human sequence (GRCh38/hg38-chr19:49,474,640-49,479,159) and a 4927 bp loxp-Neo-loxp self-deleting cassette (FIG. 1C). Successful integration was confirmed by a modification of allele (MOA) assays as described, e.g., in Valenzuela et al, supra. Probes used for the MOA assays for the loss of mouse Flt31 sequences and presence of human FLT3L sequences are depicted in the Table 6 below, with their approximate location depicted schematically in FIGS. 1C and 1D. The cassette was subsequently removed by expression of CRE recombinase (controlled by Protamine promoter) in mice (FIG. 1D).

TABLE 6

| Loss of | | |
|---|---|---|
| Allele Probe | | Coordinates |
| Name | Probe (5′ to 3′) | (GRCh38/mm10) |
| mFLT31-U | TGTGCTGGGTCCT | chr7: |
| | TATCTCCGCAT | 45,135,622- |
| | (SEQ ID NO: 27) | 45,135,645 |
| mFLT31-D | TCCCACCTCCT | chr7: |
| | GAAGGACACCT | 45,133,966- |
| | (SEQ ID NO: 28) | 45,133,987 |
| Gain of | | |
| Allele Probe | | Coordinates |
| Name | Probe (5′ to 3′) | (GRCh38/hg38) |
| hFLT3L-U | CCCATCTCCTCCG | chr19: |
| | ACTTCGCTGT | 49,475,763- |
| | (SEQ ID NO: 29) | 49,475,785 |
| hFLT3L-D | CCTGCAGGAGACC | chr19: |
| | TCCGAGCAG | 49,478,956- |
| | (SEQ ID NO: 30) | 49,478,977 |

Positively targeted ES cells were used as donor ES cells and microinjected into a pre-morula (8-cell) stage mouse embryo by the VELOCIMOUSE® method (see, e.g., U.S. Pat. Nos. 7,576,259, 7,659,442, 7,294,754, and US 2008-0078000 A1, all of which are incorporated herein by reference). The mouse embryo comprising the donor ES cells was incubated in vitro and then implanted into a surrogate mother to produce an F0 mouse fully derived from the donor ES cells. Mice with Flt31 gene deleted and humanized Flt31 inserted, were identified by genotyping using the MOA assays described above. Mice heterozygous for deletion of the Flt31 gene and gain of the humanized Flt31 gene were bred to homozygosity. These humanized Flt31 mice also comprising Rag2 gene knock-out, Il2rg gene knock-out, humanized Sirpa were bred to homozygosity for all genes.

Based on the particular design, the mice were generated that encode a chimeric membrane bound FLT3L, as depicted in FIG. 1E and labeled as “Chimeric_FLT3LG_MB”. Membrane bound mouse Flt3 ligand depicted in this Figure and labeled as “mFlt31_MB” is obtained from NCBI Reference Sequence: NP_038548.3, Uniprot: A9QW46; and membrane bound human FLT3 ligand depicted in this Figure and labeled as “hFLT3LG_MB” is obtained from NCBI Reference Sequence: NP_001191431.1, Uniprot: P49771-1; protein domains are as indicated in the Figure.

### Example 1.2: Generation of Flt3 Knock Out Mouse

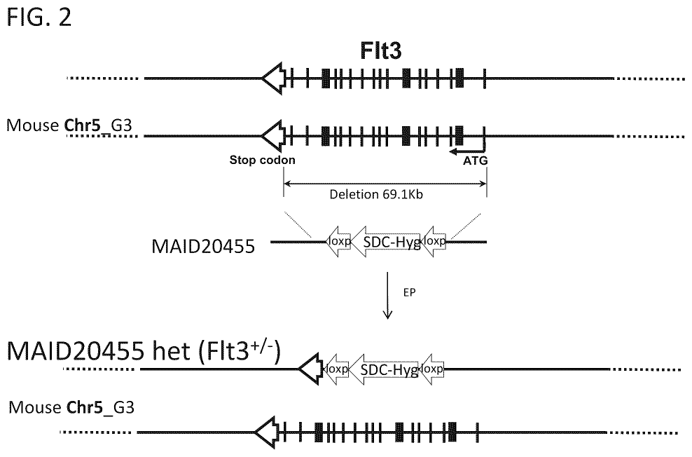

Mouse Flt3 gene was deleted in the mouse genome using VELOCIGENE® technology (see, e.g., U.S. Pat. No. 6,586,251 and Valenzuela et al. (2003) High-throughput engineering of the mouse genome coupled with high-resolution expression analysis. Nat. Biotech. 21(6): 652-659, both incorporated herein by reference). A 69.1 kb mouse genomic sequence of the mouse Flt3 gene, from the start codon ATG to the stop codon, was deleted on mouse chromosome 5 G3, between coordinates chr5: 147331171-147400265 (GRCm38 assembly). See, FIG. 2.

In detail, mouse homology arms were made by PCR amplification using BAC clone RP23-76I6 (ThermoFisher/Invitrogen) as the template, and are indicated in Table 7 below:

TABLE 7

| | | | Coordinates |
|---|---|---|---|
| Homology | 5′ primer | 3′ primer | (GRCm38 |
| Arm | (SEQ ID NO) | (SEQ ID NO) | assembly) |
| 5′; 175 bp | CCTGTGCG | TAGCGAGG | Chr5: |
| | AGGAGAGGT | AGGCCTTG | 147330999- |
| | TTGAT | GACC | 147331173 |
| | (SEQ ID | (SEQ ID | |
| | NO: 21) | NO: 22) | |
| 3′; 342 bp | GGCCTCCGG | GTTTAAAG | Chr5: |
| | AGCGCGCGG | CAGAGGTC | |
| | TA | CGGGGA | 147400266 |
| | (SEQ ID | (SEQ ID | |
| | NO: 23) | NO: 24) | 147400607 |

To make the targeting vector (designated MAID20455) from mouse BAC clone RP23-76I6, a Hygromycin (Hyg) resistance self-deleting cassette (with CRE recombinase controlled by Protamine promoter) flanked by loxp sites (loxp-hyg-loxp), replaced ˜69.1 kb mouse sequence containing the mouse Flt3 gene by bacterial homologous recombination (BHR).

The final targeting vector contained from 5′ to 3′: the 5′ mouse homology arm, the loxp-Hyg-loxp self-deleting cassette, the 3′ mouse homology arm; the chloramphenicol resistance cassette (CM; not depicted on FIG. 2); the final clone was selected based on CM/Hyg resistance.

MAID20455 targeting vector was electroporated into mouse embryonic stem (ES) comprising hSIRP-alpha (or human SIRPA), RAG2−/− IL2Rg−/− modifications. Targeted homologous recombination resulted in deletion of ˜69.1 kb of mouse sequence (GRCm38 coordinates chr5: 147331171-147400265). Successful integration was confirmed by a modification of allele (MOA) assay as described, e.g., in Valenzuela et al, supra. Primers and probes used for the MOA assay for the loss of mouse Flt3 sequences are depicted in Table 8 below. The cassette was subsequently removed by expression of CRE recombinase (controlled by Protamine promoter) in mice.

TABLE 8

| | | Coordinates |
|---|---|---|
| Probe | Probe (5′ to 3′) | (GRCm38 |
| Name | (SEQ ID NO) | assembly) |
| Flt3_U | ATGTAGGCTGACAGA | Chr5: |
| | GCAAACTAATGGG | 147399863- |
| | (SEQ ID NO: 25) | 147399890 |
| Flt3_D | TCCCAGTAAACAGGG | Chr5: |
| | TAGCTCACA | 147331770- |
| | (SEQ ID NO: 26) | 147331793 |

Positively targeted ES cells were used as donor ES cells and microinjected into a pre-morula (8-cell) stage mouse embryo by the VELOCIMOUSE® method (see, e.g., U.S. Pat. Nos. 7,576,259, 7,659,442, 7,294,754, and US 2008-0078000 A1, all of which are incorporated herein by reference). The mouse embryo comprising the donor ES cells was incubated in vitro and then implanted into a surrogate mother to produce an F0 mouse fully derived from the donor ES cells. Mice with Flt3 gene deleted were identified by genotyping using the MOA assay described above. Mice heterozygous for deletion of Flt3 gene were bred to homozygosity. Flt3 KO mice also comprising Rag2 gene knock-out, Il2rg gene knock-out, humanized Sirpa were bred to homozygosity for all genes. These mice are designated as Flt3^{-/-}, mFLT3^{-/-}, Flt3 KO, mFlt3 KO, or mFLT3 KO mice in the remaining Examples and relevant portions of the Specification. Subsequently, mice comprising humanized Flt31, Rag2 gene knock-out, Il2rg gene knock-out, and humanized Sirpa were bred to mice comprising Flt3 KO, Rag2 gene knock-out, Il2rg gene knock-out, and humanized Sirpa. Mice comprising Flt3 KO and also comprising Rag2 gene knock-out, Il2rg gene knock-out, humanized Sirpa, and humanized Flt31 are referred to herein as SRG/hFLT3L/mFLT3 KO (also referred to as “SRG hFLT3L mFLT3 KO” mice, “SRG-hFLT3L/mFLT3 KO” mice, or “hFLT3L/mFLT3 KO” mice); while mice comprising Rag2 gene knock-out, Il2rg gene knock-out, humanized Sirpa are referred to as SRG mice, and SRG mice having humanized thrombopoietin are referred to as StRG mice (these mice were used as a control, are expected to have the same level of human dendritic cell engraftment as SRG mice, and described in, e.g., U.S. Pat. No. 10,463,028, incorporated herein by reference).

### Example 1.3: Humanized Flt3l is Expressed in Genetically Engineered Mice

In order to verify that humanized Flt31 is expressed in the genetically engineered mice, blood was collected from non-engrafted StRG and SRG/hFLT3L/mFLT3 KO mice and added to serum separator tubes to harvest serum. Bone marrow aspirate was also collected from non-engrafted StRG and SRG/hFLT3L/mFLT3 KO mice. Samples were analyzed for humanized Flt31 with the human FLT3L Quantikine ELISA (R&D systems) according to manufacturer instructions. Normal human serum and bone marrow aspirate from healthy donors were used as comparative controls.

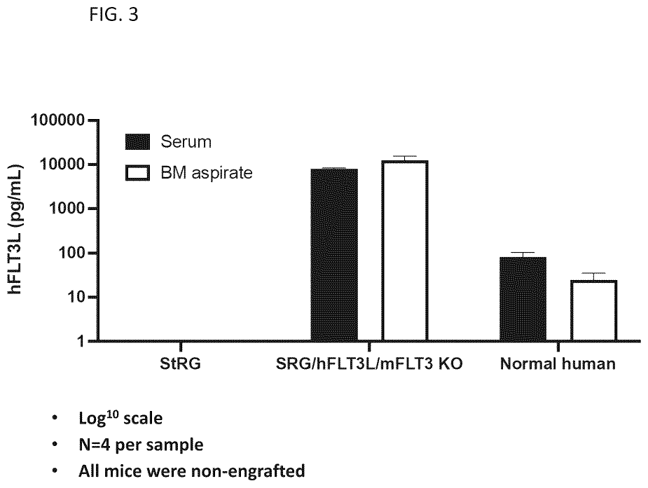

Levels of humanized Flt31 in non-engrafted SRG/hFLT3L/mFLT3 KO mice was around 10,000 μg/mL in both serum and bone marrow aspirate, whereas levels in normal healthy human serum is around 100 μg/mL and slightly less in human bone marrow aspirate (FIG. 3). The supra-physiological levels of humanized Flt31 in the SRG/hFLT3L/mFLT3 KO are probably due to the deletion of mouse FLT3 which leads to higher free levels of humanized Flt31 since there is no receptor to consume it. As expected, samples StRG mice that express only mouse Flt31 were negative for human FLT3L. Columns show mean values of n=4 per sample with standard error given.

### Example 2: Human Dendritic Cell Development in the Genetically Engineered Mice

### Example 2.1: Increased Human Myeloid and Plasmacytoid Dendritic Cells (DCs) in Engrafted Genetically Engineered Mice

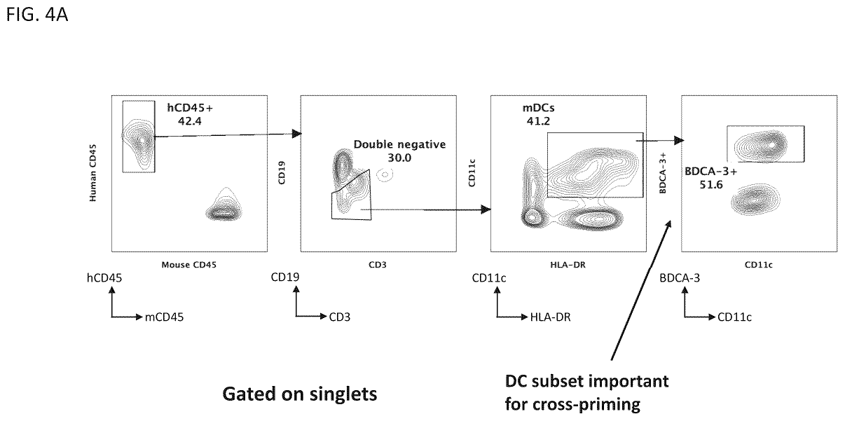

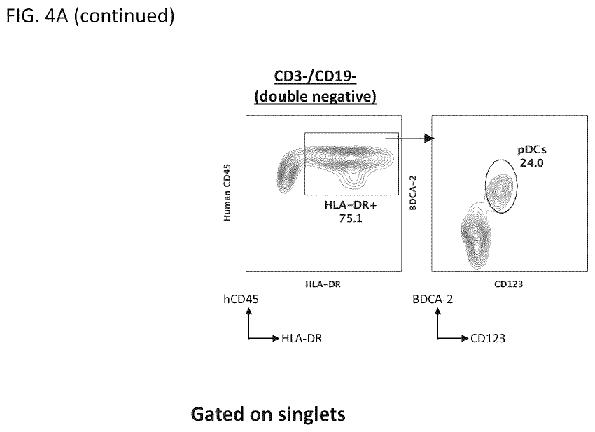

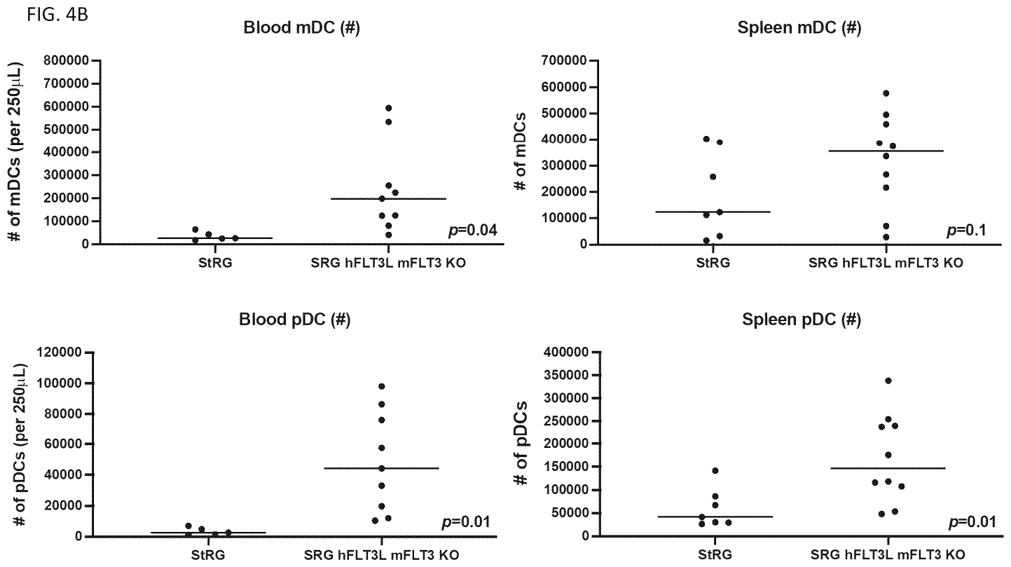

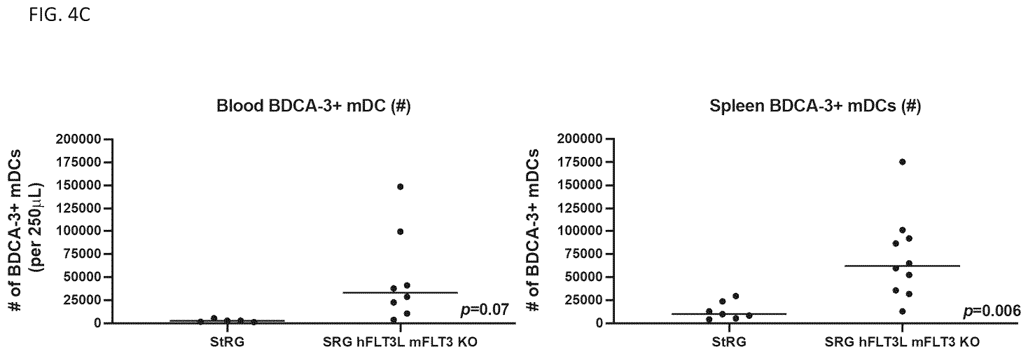

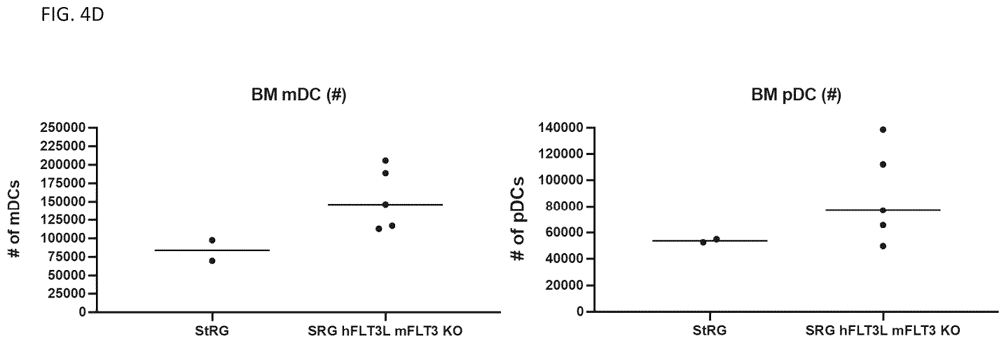

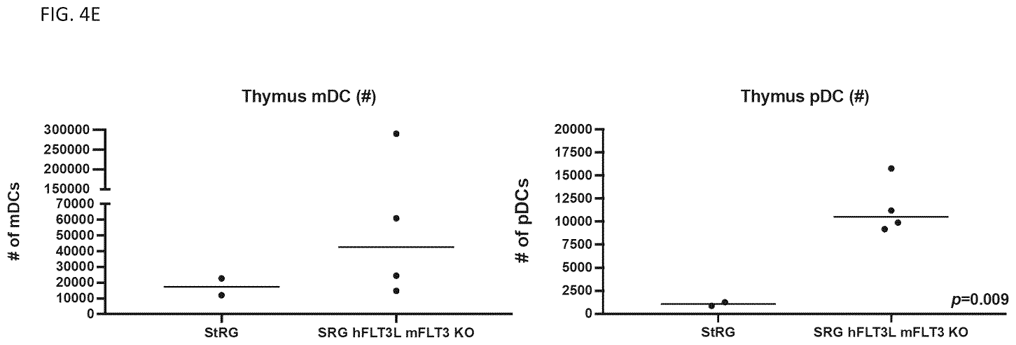

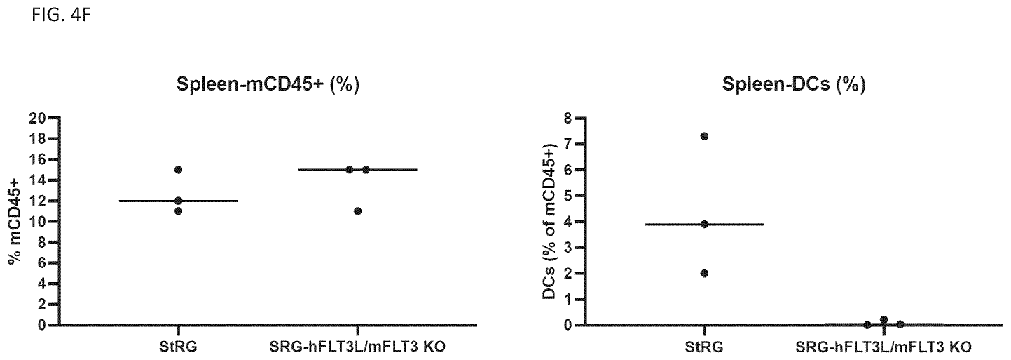

SRG/hFLT3L/mFLT3KO and StRG mouse strains were engrafted by injection of 100,000 hHSCs injected intra-hepatically into 1-5 day old pups. 10-12 weeks later, mice were bled by cardiac puncture and spleens were harvested. Single cell suspension was prepared by mechanical disruption of spleen, passage through a 70 mM mesh filter, followed by lysis of red blood cells (RBCs) in ACK lysing buffer (Gibco). Blood single cell suspension was prepared by RBC lysis in ACK lysing buffer (Gibco). All cells were resuspended in FACS buffer with mouse/human Fc block (BD Biosciences) and counted. Cells were stained for FACS analysis with the following monoclonal antibodies at a 1:50 dilution in PBS+1 mM EDTA and 2% fetal bovine serum (FACS buffer): anti-mouse CD45-APC-Cy7 (clone 30-F11; BD Biosciences), anti-human CD45-PE-Cy5.5 (clone HI30; ThermoFisher), anti-human CD19-PE-Cy7 (clone HIB19; BD Biosciences), anti-human CD3-Pacific Blue (clone 54.1; ThermoFisher), anti-human CDllc-APC (clone Bul5; Biolegend), anti-human HLA-DR-PE (clone Tu36;Biolegend), and anti-human BDCA-3-BV605 (clone M80; Biolegend), anti-human CD123 FITC (clone 6H6; Biolegend), and anti-human BDCA-2-PE-Dazzle (clone 201A; Biolegend). Cells were acquired on a BD FACSymphony A3 and analysis was done with FlowJo software. Human DC populations were analyzed from human CD45+/mouse CD45−/human CD3−/human CD19− populations. Myeloid DCs were then distinguished as human CD11c+/human HLA-DR+(FIG. 4A). Of those myeloid DCs, further analysis was done to distinguish BDCA-3+ DCs (BDCA-3+/CD11c+), a subset considered the most potent at cross-presentation of exogenous antigen on MHC class I (FIG. 4A). Plasmacytoid DCs, highest producers of type I IFN, were distinguished from human CD45+/mouse CD45−/human CD3−/human CD19−/human HLA-DR+ population by co-expression of BDCA-2 and CD123 (FIG. 4A).

hCD45+ engraftment was comparable between the two mouse strains but the SRG/hFLT3L/mFLT3 KO had increased myeloid and plasmacytoid DCs in the blood and spleen (FIG. 4B). Additionally, SRG/hFLT3L/mFLT3 KO mice have increased BDCA-3+ myeloid DCs in their blood and spleen upon hHSC engraftment (FIG. 4C). Data represents 3 HSCs donors matched between StRG and SRG/hFLT3L/mFLT3 KO mice with individual data points per mouse and means given. Statistical analysis was done with unpaired student t test.

Bone marrow (BM) was prepared from 10-12 week hHSC-engrafted mice by harvesting femurs/tibia which were then ground and ground bones passaged through a 70 mM mesh filter followed lysis of RBCs with ACK lysing buffer (Gibco). Cells were then FACS stained and DC analysis performed as previously described.

SRG/hFLT3L/mFLT3 KO have comparable engraftment to HSC donor-matched StRG mice but increased human myeloid and plasmacytoid DCs in the BM (FIG. 4D). Individual data points per mouse and mean are given. Statistical analysis was done with unpaired student t test.

Thymus was also harvested from engrafted mice and then mechanically disrupted. Single cell suspension was prepared by passage through a 70 mM mesh filter and lysis of RBCs with ACK lysing buffer (Gibco). Cells were then FACS stained and DC analysis performed as previously described. SRG/hFLT3L/mFLT3 KO have comparable engraftment to HSC donor-matched StRG mice but increased human myeloid and plasmacytoid DCs in the thymus (FIG. 4E). Individual data points per mouse and mean are given. Statistical analysis was done with unpaired student t test.

We theorized that human DC development in hHSC engraftment is potentiated by loss of murine DCs, thus removing a potentially competitive cellular niche. To that end, spleens from engrafted mice were also FACS stained with the following monoclonal antibodies: anti-mouse CD45-APC-Cy7 (clone 30-F11; BD Biosciences), anti-mouse CD11c APC (clone N418; Biolegend) and anti-mouse MHC class II I-A^{bid}-PE (clone M5/114.15.2; BD Biosciences). In engrafted SRG/hFLT3L/mFLT3 KO, there is a significant loss of murine DCs (mouse CD45+/CD11c+/MHC Class II+) indicating that by deleting mouse Flt3, mouse DCs are significantly reduced (FIG. 4F). Individual data points per mouse and mean are given. Statistical analysis was done with unpaired student t test.

### Example 2.2: Increased Human DC Development Requires Humanization of Flt3l and Deletion of Murine FLT3/FLK2

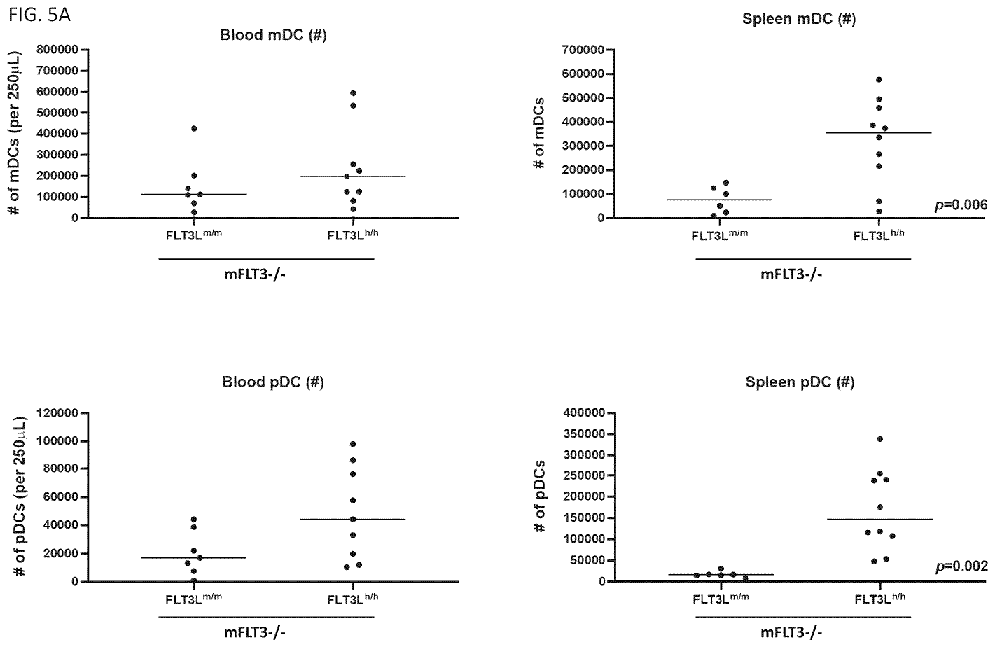

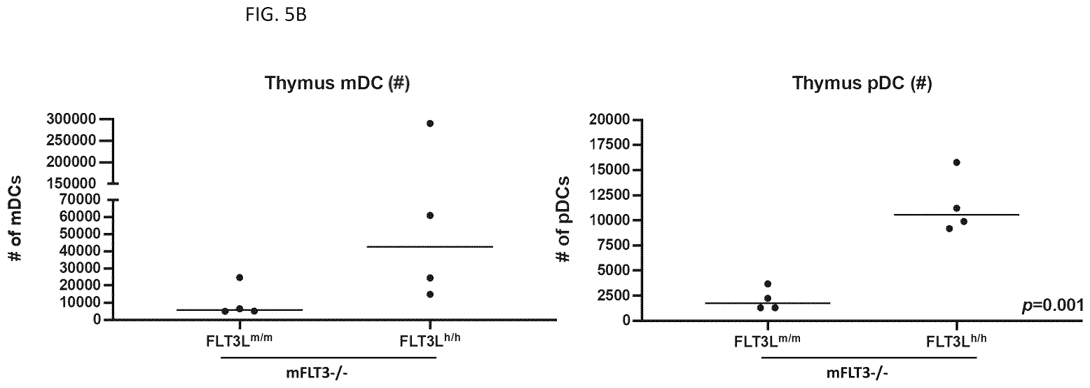

It has been reported that mice with deleted FLT3 have increased human DC development upon human hematopoietic stem cell (hHSC) engraftment. However, increased human DCs required repeated injection of large doses of human FLT3L (Li et al. (2016) Eur J Immunol 46:1291-1299). Our model has receptor deletion with concomitant humanization of Flt31, such that Flt31 isoforms are expressed continuously at physiologically-relevant levels. To address whether this additional humanization is necessary, we analyzed hHSC-engrafted SRG mice with mFlt3 KO (FLT3L^{m/m}FLT3−/−) against SRG mice with mFlt3 KO and humanized Flt31 (FLT3L^{h/h}mFLT3−/−). Blood, spleen, and thymus was prepared from 10-12 weeks engrafted mice, and human DC populations were analyzed as previously described. Human mDC and pDC populations were increased in the blood and spleen of hHSC-engrafted mice with humanized FLT3L/mFLT3 KO (FLT3L^{h/h}mFLT3−/−) relative to HSC donor-matched mice with just mFlt3 KO (FLT3L^{m/m}FLT3−/−) (FIG. 5A). Furthermore, the same pattern can be observed in the thymus (FIG. 5B). Thus, we conclude that optimal human DC development in this novel human immune system strain requires both murine Flt3/FLK2 deletion and humanized Flt31. Individual data points per mouse and mean are given.

### Example 3: Human T Cell Development in the Genetically Engineered Mice

Since DCs are critical regulators of T cell activation, human T cell phenotype in hHSC-engrafted SRG/hFLT3L/mFLT3 KO versus StRG mice was evaluated. Human T cells in human immune system mouse models have a dysregulated phenotype as evidenced by higher expression of checkpoint inhibitors such as PD-1. In normal human T cells, around 5% or less of T cells are PD-1+ whereas this is consistently higher in human T cells resulting from hHSC engraftment of various previously characterized human immune system mice.

StRG and SRG/hFLT3L/mFLT3 KO mice were engrafted as previously described and retro-orbitally bled at 10-12 weeks post engraftment. RBCs were lysed with ACK lysing buffer (Gibco) and cells stained as previously described with the following monoclonal antibodies: anti-mouse CD45-APC-Cy7 (clone 30-F11; BD Biosciences), anti-human CD45-PE-Cy5.5 (clone HI30; ThermoFisher), anti-human CD19-FITC (clone HIB19; BD Biosciences), anti-human CD3-Pacific Blue (clone S4.1; ThermoFisher), anti-human CD4 BUV496 (clone SK3; BD Biosciences), anti-human CD8-APC (clone SK1; Biolegend) and anti-human PD1-BV605 (clone EH12; BD Biosciences).

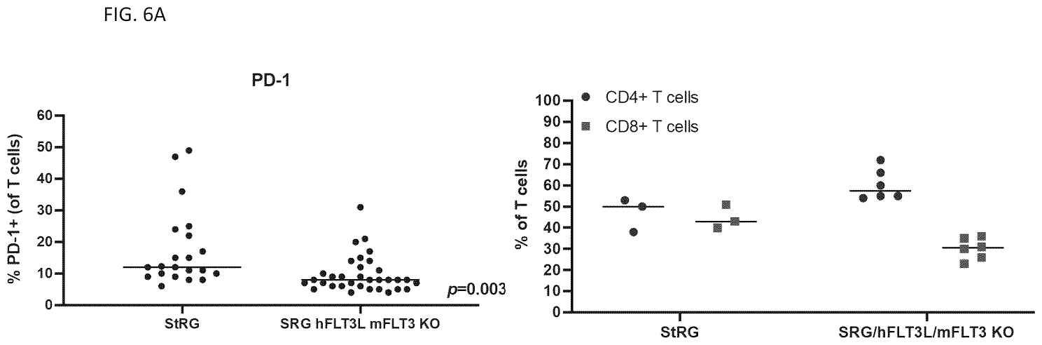

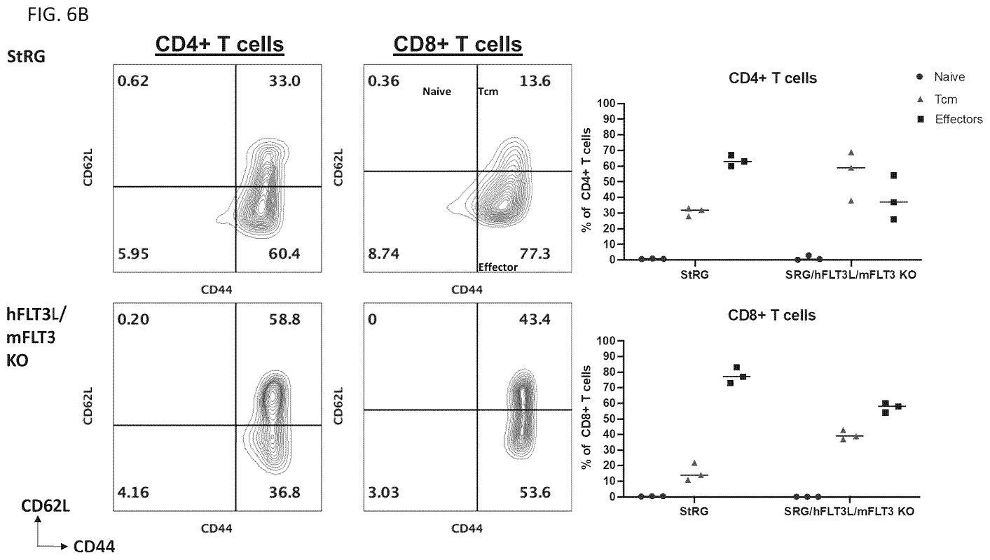

CD3+ T cells in the blood expressed significantly less PD-1 in hHSC-engrafted SRG/hFLT3L/mFLT3 KO than in StRG mice (FIG. 6A). Additionally, assessment of splenic T cells indicated that there is a higher percentage of CD4+ T cells than CD8+ T cells in hHSC-engrafted SRG/hFLT3L/mFLT3 KO mice (FIG. 6A). This would indicate a normalization of T cell subsets since normal human distribution of CD4/CD8 T cells is 60/40 as is observed in SRG/hFLT3L/mFLT3 KO (FIG. 6A), whereas in StRG mice the ratio is more 50/50. Individual data points per mouse and mean are given. All analysis was done with hHSC donor-matched StRG and SRG/hFLT3L/mFLT 3KO and statistical analysis was done with unpaired student t test.

To evaluate activation status of human T cells in the different human immune system mouse strains, spleen cells from hHSC-engrafted StRG and SRG/hFLT3L/mFLT3 KO mice were FACS stained with the following monoclonal antibodies: anti-mouse CD45-APC-Cy7 (clone 30-F11; BD Biosciences), anti-human CD45-PE-Cy5.5 (clone HI30; ThermoFisher), anti-human CD3-Pacific Blue (clone S4.1; ThermoFisher), anti-human CD4 BUV496 (clone SK3; BD Biosciences), anti-human CD8-APC (clone SK1; Biolegend), anti-human CD44-PE (clone C44Mab-5;Biolegend) and anti-human CD62L-FITC (clone SK11; BD Biosciences). The following combinations of CD44 and CD62L expression denote different T cell subsets: CD44−/CD62L+ (Naïve T cells), CD44+/CD62L− (Effector cells), and CD44+/CD62L+ (Central memory T cells; Tcm). It is generally considered that T cells in human immune system mouse models are hyporesponsive to antigen stimulation, and memory responses are very sub-optimal. However, greater CD4+ and CD8+ T cells in hHSC-engrafted SRG/hFLT3L/mFLT3 KO were Tcm than in HSC donor-matched StRG (FIG. 6B). Individual data points per mouse and mean are given.

### Example 4: Characterization of Dendritic Cell Populations in the Genetically Engineered Mice

In order to compare human DCs in hHSC-engrafted SRG/hFLT3L/mFLT3 KO mice to DCs from healthy human donors, bulk human DCs were isolated from spleen/blood of engrafted mice and blood form healthy human donors using EasySep Human Pan-DC pre-enrichment kit according to manufacturer instructions (StemCell Technologies). Additionally, bulk human DCs were also enriched from hHSC-engrafted SRG/hFLT3L/mFLT3 KO BM and healthy human BM. Isolated DCs from hHSC-engrafted StRG were also used as comparative controls, albeit with too few cells isolated for confidence in analysis. 3 different donors for human blood, BM, and HSC donor for engraftment were used in the evaluation.

Isolated DCs were resuspended in PBS with 0.04% BSA (˜6000 cells) and were loaded on a Chromium Single Cell Instrument (10× Genomics). RNA-seq were prepared using Chromium Single Cell v1.0 5′ Library, Gel Beads & Multiplex Kit (10× Genomics). For RNA-seq libraries, Cell Ranger Single-Cell Software Suite (10× Genomics, v2.2.0) was used to perform sample demultiplexing, alignment, filtering, and UMI counting. The mouse mm10 genome assembly and RefSeq gene model for mouse were used for the alignment. Analysis was then performed to cluster DC populations based on transcriptional profile (data not shown).

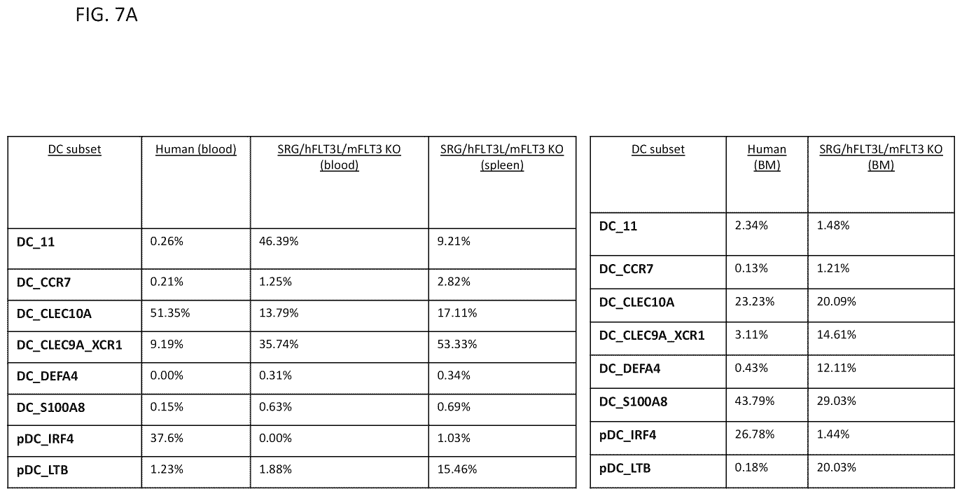

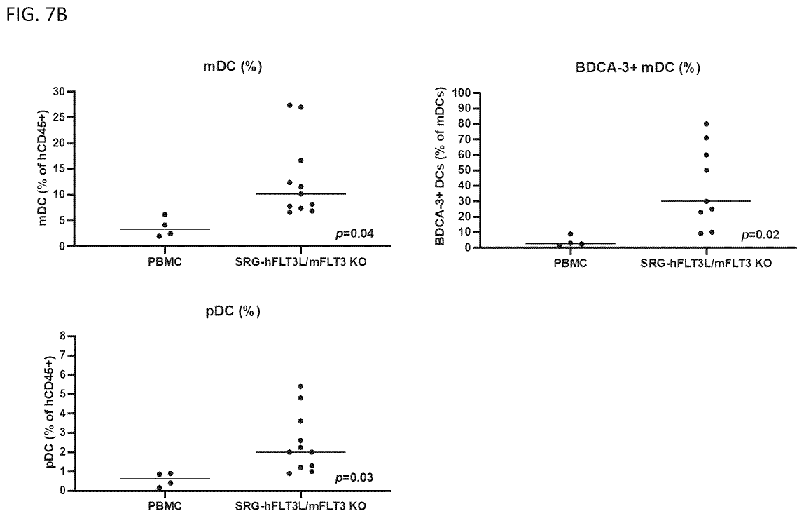

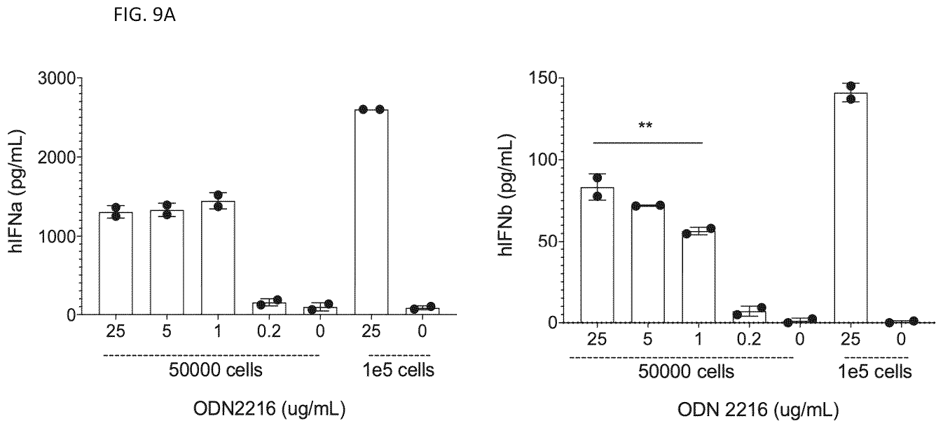

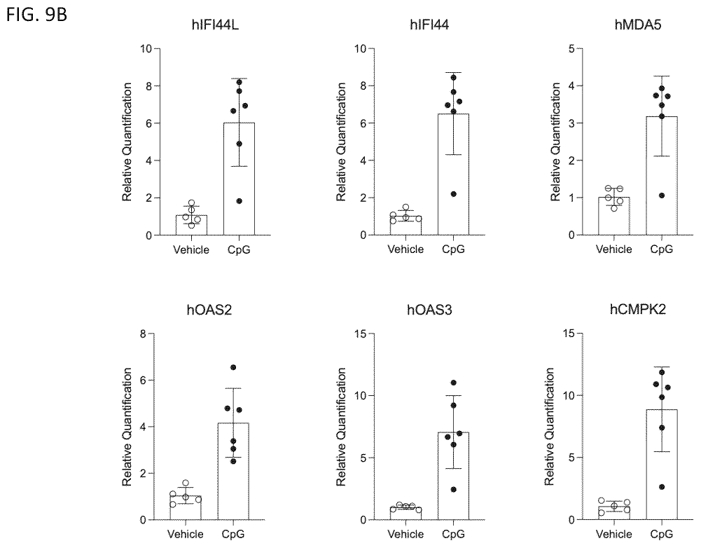

Key transcriptional differences between DC populations in spleen/blood of hHSC-engrafted SRG/hFLT3L/mFLT3 KO mice and healthy human blood were noted. hHSC-engrafted SRG/hFLT3L/mFLT3 KO had a lower percentage of Clec10a myeloid DCs relative to healthy human donors (FIG. 7A). Additionally, the transcriptional profile of plasmacytoid DCs in healthy human donors is denoted by IRF4 expression, whereas this transcriptional signature is reduced in plasmacytoid DCs from hHSC-engrafted SRG/hFLT3L/mFLT3 KO mice (FIG. 7A). Since IRF4 is a critical regulator of type I IFN production by plasmacytoid DCs, further work was done to evaluate type I IFN production by plasmacytoid DCs from hHSC-engrafted mice (FIGS. 9A and 9B). Finally, hHSC-engrafted SRG/hFLT3L/mFLT3 KO mice have higher percentage of XCR1+/Clec9a+ DCs in spleen/blood compared to healthy human DCs (FIG. 7A). Notably, this population is the BDCA-3+DC subset and FACS analysis of blood from hHSC-engrafted SRG/hFLT3L/mFLT3 KO mice showed that indeed this population is over-represented compared to DCs from human peripheral blood mononuclear cells (PBMCs) (FIG. 7B). Additionally, there is a greater percentage of human myeloid and plasmacytoid DCs in hHSC-engrafted SRG/hFLT3L/mFLT3 KO mice than in human PBMCs (FIG. 7B). BDCA-3+/Clec9a+/XCR1+ myeloid DC has been characterized as critical DC subset for cross-priming and activation of cytotoxic CD8+ T cell lymphocytes (CTLs) against tumor cells, viral pathogens, and the establishment of immunological tolerance in the periphery (Crozat & Dalod (2011) Med Sci (Paris) 27:25-28; Cui et al. (2021) Am J Physiol Lung Cell Mol Physiol 320:L193-L204; Schreibelt et al. (2012) Blood 119: 2284-2292; van der Aa, E., van Montfoort, N. & Woltman, A. M. (2015) Semin Cell Dev Biol 41:39-48). This subset of DCs is superior at cross-presentation due to the retargeting of antigens from early endocytic vesicles to late endosomes/lysosomes and subsequently to the cytosol to be presented on the MHC-I pathway (Cohn et al. (2013) J. Exp. Med. 210(5):1049-63 and Savina et al. (2009) Immunity 30(4):544-55). Indeed, this DC subset is an attractive target for immune-therapy that looks to stimulate T cell responses to tumors and pathogens (Crozat & Dalod (2011) Med Sci (Paris) 27:25-28; Schreibelt et al. (2012) Blood 119: 2284-2292; van der Aa, E., van Montfoort, N. & Woltman, A. M. (2015) Semin Cell Dev Biol 41:39-48; Masterman et al (2020) J Immunother Cancer 8; Pearson et al. (2020) Clin Transl Immunology 9:e1141; Tullett et al. (2016) JCI Insight 1: e87102). Thus, our SRG/hFLT3L/mFLT3 KO human immune system strain is an attractive model for testing potential immunotherapies aimed at cross-priming due to the enhanced presentation of peptides by this DC subset.

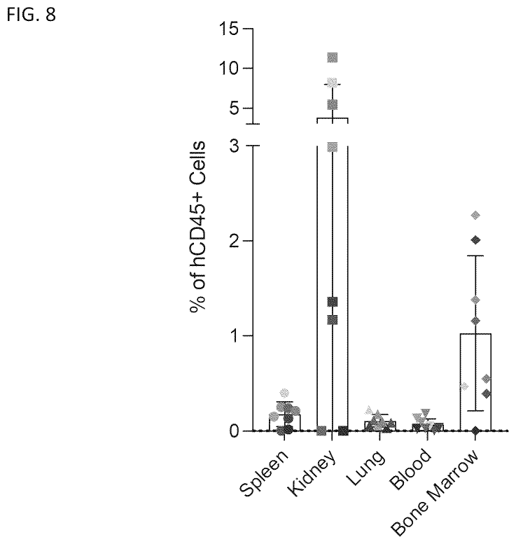

Plasmacytoid DCs (pDCs) are critical type I IFN producers in viral infection and have also been implicated in autoimmune diseases such as lupus (Furie et al. (2022) N Engl J Med 387:894-904; Laurent et al. (2022) Sci Immunol 7:eadd4906; Li et al. (2020) Front Pharmacol 11: 8; Palucka (2005) Proc Natl Acad Sci USA 102:3372-3377; Sakata et al. (2018) Front Immunol 9:1957; Sprow et al. (2022) Front Med (Lausanne) 9:968323; Takagi et al. (2016) Sci Rep 6:24477; Werth et al. (2022) N Engl J Med 387:321-331; Zhang et al. (2017) Proc Nat Acad Sci USA 114:1988-1993). To test whether human pDCs were present in various organs in SRG/hFLT3L/mFLT3 KO human immune system mice, spleen, kidney, lung, blood, and bone marrow were removed from SRG/hFLT3L/mFLT3 KO human immune system mice and processed for single cell suspensions. Human pDCs were evaluated by flow cytometry, based on gating as follows: lymphocytes, singlets, live, mouse CD45−, human CD45+, human CD3−, human CD16−, human CD19−, human CD123+, human HLA-DR+, human ILT7+, human BDCA2+(FIG. 8). Data shown are plotted as pDCs as a percentage of total live human CD45+ cells and individual mice are graphed. This data indicated that human pDCs can be identified in blood, lymphoid organs, and other tissue sites in SRG/hFLT3L/mFLT3 KO human immune system mice.

To test whether human pDCs in these mice retain functionality, human pDCs were isolated from the SRG/hFLT3L/mFLT3 KO human immune system mice and stimulated in vitro to determine whether they are able to produce Type I Interferon (IFN) in response to stimulation. Single cell suspensions from spleen and bone marrow were obtained by tissue dissociation. Murine cells were removed by staining for APC-conjugated mouse CD45 and mouse Ter119, followed by magnetic separation. The human pDCs were subsequently isolated using the EasySep human pDC enrichment kit (Stem cell). pDCs were plated at the indicated cell numbers and ODN2216 (Invivogen), a class A stimulatory CpG ODN, was added at the indicated concentrations. Cells were incubated with stimulation for 18 hours. Supernatants were harvested and evaluated by ELISA for human IFNα or IFNβ respectively. As shown in FIG. 9A, human pDCs isolated from SRG/hFLT3L/mFLT3 KO human immune system mice respond to TLR9 stimulation and produce Type I IFNs. Further, it was determined that human pDCs in these mice respond to ODN2216 CpG stimulation in vivo and produce human Type I IFN as measured by induction of Type I IFN signature genes (FIG. 9B). Specifically, mice were treated intravenously with 5 ug ODN2216 delivered with the InVivoJet delivery system (PolyPlus). Blood was obtained after 6 hours and processed to isolate RNA. qPCR was run to evaluate gene expression of the indicated genes and analyzed to determine the relative quantification based on standard 2^{ΔΔct} analysis.

Our data demonstrated that human plasmacytoid DCs in hHSC-engrafted SRG/hFLT3L/mFLT3 KO mice were able to elicit Type I IFN response. Therefore, our SRG/hFLT3L/mFLT3 KO human immune system mouse is a great model for testing modulators of plasmacytoid DC function.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

Also incorporated by reference in their entirety are any polynucleotide and polypeptide sequences which reference an accession number correlating to an entry in a public database, such as those maintained by The Institute for Genomic Research (TIGR) on the World Wide Web at tigr.org and/or the National Center for Biotechnology Information (NCBI) on the World Wide Web at ncbi.nlm.nih.gov. A person of ordinary skill in the art would understand that the sequence databases disclosed above or those known in the art are periodically updated to publish corrected sequences. Incorporated by reference in their entirety are such corrected sequences.

### EQUIVALENTS

Those skilled in the art will recognize, or be able to ascertain using no more than routine experimentation, many equivalents to the specific embodiments of the invention described herein. Such equivalents are intended to be encompassed by the following claims.

What is claimed is:

1.-98. (canceled)

99. A genetically modified rodent cell, comprising:
(i) a homozygous null mutation in Rag2 gene;
(ii) a homozygous null mutation in IL2rg gene;
(iii) a homozygous null mutation in the rodent FMS-like tyrosine kinase 3 (Flt3) gene; and
(iv) a Flt3 ligand (Flt31) gene that comprises a rodent portion and a human portion operably linked to a Flt31 promoter.

100. The genetically modified rodent cell of claim 99 comprising a homozygous null mutation in Rag1 gene.

101. The genetically modified rodent cell of claim 99, wherein the null mutation in the rodent Flt3 gene is a deletion of the full Flt3 endogenous coding sequence.

102. The genetically modified rodent cell of claim 99, wherein the rodent portion of the Flt31 gene comprises exon 1, a non-coding portion of exon 2, and exons downstream of exon 6 of a rodent Flt31 gene.

103. The genetically modified rodent cell of claim 99, wherein the human portion of the Flt31 gene comprises a signal peptide-coding portion of exon 2, and exons 3-6 of a human FLT3L gene.

104. The genetically modified rodent cell of claim 99, wherein the Flt31 gene comprises rodent exon 1, rodent non-coding portion of exon 2, human signal peptide-coding portion of exon 2, human exons 3-6, and rodent exons 7-9.

105. The genetically modified rodent cell of claim 99, wherein the Flt31 gene encodes a chimeric membrane bound FLT3L comprising a signal peptide and cytokine-like core domain of a human FLT3L polypeptide, and a stalk region, a transmembrane domain, and a cytoplasmic tail of a rodent Flt31 polypeptide.

106. The genetically modified rodent cell of claim 99, wherein the rodent cell expresses both soluble and membrane-bound forms of the Flt31 polypeptide.

107. The genetically modified rodent cell of claim 99, wherein the rodent portion of the Flt31 gene is an endogenous rodent portion of the Flt31 gene and the rodent Flt31 gene is an endogenous rodent gene.

108. The genetically modified rodent cell of claim 99, wherein the Flt31 promoter is an endogenous rodent promoter.

109. The genetically modified rodent cell of claim 99, wherein the genetically modified rodent cell further comprises a nucleic acid that encodes a human or humanized SIRPA polypeptide, and wherein the nucleic acid is operably linked to a Sirpa promoter.

110. The genetically modified rodent cell of claim 99, wherein the genetically modified rodent cell further comprises: (1) a nucleic acid that encodes a human GM-CSF protein operably linked to a GM-CSF promoter; and/or (2) a nucleic acid that encodes a human IL3 protein operably linked to a IL3 promoter.

111. The genetically modified rodent cell of claim 99, wherein the rodent cell is a rat cell or a mouse cell.

112. The genetically modified rodent cell of claim 99, wherein the genetically modified rodent cell is a rodent embryonic stem (ES) cell.

113. A genetically modified rodent, comprising:
(i) a homozygous null mutation in Rag2 gene;
(ii) a homozygous null mutation in IL2rg gene;
(iii) a homozygous null mutation in the rodent FMS-like tyrosine kinase 3 (Flt3) gene; and
(iv) a Flt3 ligand (Flt31) gene that comprises a rodent portion and a human portion operably linked to a Flt31 promoter.

114. A method of identifying an agent that modulates a function of a human dendritic cell, comprising:
a. administering the candidate agent to a genetically modified rodent of claim 113; and
b. determining whether the candidate agent modulates the function of the human dendritic cell in the rodent.

115. The method of claim 114, wherein the human dendritic cell is a myeloid dendritic cell (mDC) or a plasmacytoid dendritic cell (pDC).

116. The method of claim 114, wherein the function of the human dendritic cell is selected from a group consisting of phagocytosis, cytokine production, cross-presentation of exogenous antigens, and activation of cytotoxic CD8+ T cell lymphocytes (CTLs).

117. A method for assessing therapeutic efficacy of a drug to stimulate a T cell response to a target cell, the method comprising:
a. administering a drug candidate to a genetically modified rodent of claim 113, wherein the genetically modified rodent comprises the target cell; and
b. measuring the T cell response to the target cell in the rodent to assess the therapeutic efficacy of the drug candidate.

118. The method of claim 117, wherein the target cell is selected from the group consisting of a tumor cell, a virally-infected cell, a bacterially-infected cell, a bacterial cell, a fungal cell, and a parasitic cell.

119. A method for assessing therapeutic efficacy of a drug in treating an autoimmune disease, the method comprising:
a. administering the candidate agent to a genetically modified rodent of claim 113; and
b. determining whether the agent treats the autoimmune disease in the rodent.

120. A method of making a rodent embryonic stem cell, comprising genetically engineering the rodent embryonic stem cell so that the rodent embryonic stem cell has a genome that comprises:
(i) a homozygous null mutation in Rag2 gene;
(ii) a homozygous null mutation in IL2rg gene;
(iii) a homozygous null mutation in the rodent FMS-like tyrosine kinase 3 (Flt3) gene; and
(iv) a Flt3 ligand (Flt31) gene that comprises a rodent portion and a human portion operably linked to a Flt31 promoter.

121. A rodent embryo comprising the rodent embryonic stem cell of claim 112.

122. A method of making a rodent comprising in its genome: (i) a homozygous null mutation in Rag2 gene; (ii) a homozygous null mutation in IL2rg gene; (iii) a homozygous null mutation in the rodent FMS-like tyrosine kinase 3 (Flt3) gene; and (iv) a Flt3 ligand (Flt31) gene that comprises a rodent portion and a human portion operably linked to a Flt31 promoter, the method comprising steps of:
(a) obtaining a rodent embryonic stem cell of claim 112; and
(b) creating a rodent using the rodent embryonic cell of (a).

123. A method of making a rodent comprising in its genome: (i) a homozygous null mutation in Rag2 gene; (ii) a homozygous null mutation in IL2rg gene; (iii) a homozygous null mutation in the rodent FMS-like tyrosine kinase 3 (Flt3) gene; and (iv) a Flt3 ligand (Flt31) gene that comprises a rodent portion and a human portion operably linked to a Flt31 promoter, the method comprising modifying the genome of the rodent so that it comprises: (i) a homozygous null mutation in Rag2 gene; (ii) a homozygous null mutation in IL2rg gene; (iii) a homozygous null mutation in the rodent FMS-like tyrosine kinase 3 (Flt3) gene; and (iv) a Flt3 ligand (Flt31) gene that comprises a rodent portion and a human portion operably linked to a Flt31 promoter.


SEQUENCE LISTING (32 sequences)

SEQ ID NO: 1 - DNA, 65 bp, Mus musculus
  misc_feature 1..65: note = Mouse Flt3l coding sequence (NM_013520.4), exon 1
  source 1..65: mol_type = genomic DNA
ctttcactttcggtctttggctgtcatccagctttggctttccacacccagctggggcaagcctg

SEQ ID NO: 2 - DNA, 34 bp, Mus musculus
  misc_feature 1..34: note = Mouse Flt3l coding sequence (NM_013520.4), non-coding portion of exon 2
  source 1..34: mol_type = genomic DNA
aacctgtcacaggcatgaggggtccccggcagag

SEQ ID NO: 3 - DNA, 185 bp, Mus musculus
  misc_feature 1..185: note = Mouse Flt3l coding sequence (NM_013520.4), exon7
  source 1..185: mol_type = genomic DNA
actcctccaccctgctgcccccaaggagtcccatagccctagaagccacggagctcccagagcctcggcccaggcagctgttgctcctgctgctgctgctgctgcctctcacactggtgctgctggcagccgcctggggccttcgctggcaaagggcaagaaggaggggggagctccaccctggg

SEQ ID NO: 4 - DNA, 69 bp, Mus musculus
  misc_feature 1..69: note = Mouse Flt3l coding sequence (NM_013520.4), exon8
  source 1..69: mol_type = genomic DNA
gtgcccctcccctcccatccctaggatgcgagccttgtgcatcgttgactcagccagggtcttatctcg

SEQ ID NO: 5 - DNA, 244 bp, Mus musculus
  misc_feature 1..244: note = Mouse Flt3l coding sequence (NM_013520.4), exon9
  source 1..244: mol_type = genomic DNA
agttgggaaccaaaacaaggaacaagctaggcaagtgctgtgctgagttacatccccagcccagaggacacactgtctgggtatggcgatggacactgtaattccagtgcttctggattggacatgctgaaactggaggatactgactttaagaaaaacagaaaggaagaaggggggtgcgttgtagccctgtgtggttgcttttgtcacctccaccccttaaaataaaagcaactgctgggta

SEQ ID NO: 6 - DNA, 33 bp, Homo sapiens
  misc_feature 1..33: note = Human FLT3L coding sequence (NM_001204502.2) , signal peptide-coding portion of exon 2
  source 1..33: mol_type = genomic DNA
atgacagtgctggcgccagcctggagcccaaca

SEQ ID NO: 7 - DNA, 111 bp, Homo sapiens
  misc_feature 1..111: note = Human FLT3L coding sequence (NM_001204502.2) , exon3
  source 1..111: mol_type = genomic DNA
acctatctcctcctgctgctgctgctgagctcgggactcagtgggacccaggactgctccttccaacacagccccatctcctccgacttcgctgtcaaaatccgtgagctg

SEQ ID NO: 8 - DNA, 54 bp, Homo sapiens
  misc_feature 1..54: note = Human FLT3L coding sequence (NM_001204502.2) , exon4
  source 1..54: mol_type = genomic DNA
tctgactacctgcttcaagattacccagtcaccgtggcctccaacctgcaggac

SEQ ID NO: 9 - DNA, 144 bp, Homo sapiens
  misc_feature 1..144: note = Human FLT3L coding sequence (NM_001204502.2) , exon5
  source 1..144: mol_type = genomic DNA
gaggagctctgcgggggcctctggcggctggtcctggcacagcgctggatggagcggctcaagactgtcgctgggtccaagatgcaaggcttgctggagcgcgtgaacacggagatacactttgtcaccaaatgtgcctttcag

SEQ ID NO: 10 - DNA, 139 bp, Homo sapiens
  misc_feature 1..139: note = Human FLT3L coding sequence (NM_001204502.2) , exon6
  source 1..139: mol_type = genomic DNA
cccccccccagctgtcttcgcttcgtccagaccaacatctcccgcctcctgcaggagacctccgagcagctggtggcgctgaagccctggatcactcgccagaacttctcccggtgcctggagctgcagtgtcagcccg

SEQ ID NO: 11 - protein, 229 aa, synthetic construct
  REGION 1..229: note = Synthetic: Humanized Flt3l Protein
MTVLAPAWSPTTYLLLLLLLSSGLSGTQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLLPPRSPIALEATELPEPRPRQLLLLLLLLLPLTLVLLAAAWGLRWQRARRRGELHPGVPLPSHP

SEQ ID NO: 12 - protein, 508 aa, synthetic construct
  REGION 1..508: note = Synthetic: Humanized Sirpa Protein
MEPAGPAPGRLGPLLLCLLLSASCFCTGVAGEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAADNNATHNWNVFIGVGVACALLVVLLMAALYLLRIKQKKAKGSTSSTRLHEPEKNAREITQIQDTNDINDITYADLNLPKEKKPAPRAPEPNNHTEYASIETGKVPRPEDTLTYADLDMVHLSRAQPAPKPEPSFSEYASVQVQRK

SEQ ID NO: 13 - protein, 21 aa, synthetic construct
  REGION 1..21: note = Synthetic: 5 ; 260bp
GGGTTCTTAGAAGAGGAGATG

SEQ ID NO: 14 - protein, 20 aa, synthetic construct
  REGION 1..20: note = Synthetic: 5 ; 260bp
GGTATACGCACATTTGGGCT

SEQ ID NO: 15 - protein, 24 aa, synthetic construct
  REGION 1..24: note = Synthetic: 3 ; 230bp
GTGTGAGTGTTTTACCTACATGCC

SEQ ID NO: 16 - protein, 22 aa, synthetic construct
  REGION 1..22: note = Synthetic: 3 ; 230bp
GCCTGCATAATGAGACCCTATC

SEQ ID NO: 17 - protein, 20 aa, synthetic construct
  REGION 1..20: note = Synthetic: 5 ; 382bp
TTGGCTGTCATCCAGCTTTG

SEQ ID NO: 18 - protein, 20 aa, synthetic construct
  REGION 1..20: note = Synthetic: 5 ; 382bp
CTCTGCCGGGGACCCCTCAT

SEQ ID NO: 19 - protein, 24 aa, synthetic construct
  REGION 1..24: note = Synthetic: 3 ; 325bp
GTGTGAGTGTTTTACCTACATGCC

SEQ ID NO: 20 - protein, 22 aa, synthetic construct
  REGION 1..22: note = Synthetic: 3 ; 325bp
AAGGAAAGGAAGCCAGATGTTC

SEQ ID NO: 21 - DNA, 22 bp, synthetic construct
  misc_feature 1..22: note = Synthetic: 5 ; 175bp
  source 1..22: mol_type = other DNA
cctgtgcgaggagaggtttgat

SEQ ID NO: 22 - DNA, 20 bp, synthetic construct
  misc_feature 1..20: note = Synthetic: 5 ; 175bp
  source 1..20: mol_type = other DNA
tagcgaggaggccttggacc

SEQ ID NO: 23 - DNA, 20 bp, synthetic construct
  misc_feature 1..20: note = Synthetic: 3 ; 342bp
  source 1..20: mol_type = other DNA
ggcctccggagcgcgcggta

SEQ ID NO: 24 - DNA, 22 bp, synthetic construct
  misc_feature 1..22: note = Synthetic: 3 ; 342bp
  source 1..22: mol_type = other DNA
gtttaaagcagaggtccgggga

SEQ ID NO: 25 - DNA, 28 bp, synthetic construct
  misc_feature 1..28: note = Synthetic: Flt3_U
  source 1..28: mol_type = other DNA
atgtaggctgacagagcaaactaatggg

SEQ ID NO: 26 - DNA, 24 bp, synthetic construct
  misc_feature 1..24: note = Synthetic: Flt3_D
  source 1..24: mol_type = other DNA
tcccagtaaacagggtagctcaca

SEQ ID NO: 27 - DNA, 24 bp, synthetic construct
  misc_feature 1..24: note = Synthetic: mFLT3l-U
  source 1..24: mol_type = other DNA
tgtgctgggtccttatctccgcat

SEQ ID NO: 28 - DNA, 22 bp, synthetic construct
  misc_feature 1..22: note = Synthetic: mFLT3l-D
  source 1..22: mol_type = other DNA
tcccacctcctgaaggacacct

SEQ ID NO: 29 - DNA, 23 bp, synthetic construct
  misc_feature 1..23: note = Synthetic: hFLT3L-U
  source 1..23: mol_type = other DNA
cccatctcctccgacttcgctgt

SEQ ID NO: 30 - DNA, 22 bp, synthetic construct
  misc_feature 1..22: note = Synthetic: hFLT3L-D
  source 1..22: mol_type = other DNA
cctgcaggagacctccgagcag

SEQ ID NO: 31 - protein, 232 aa, Mus musculus
  REGION 1..232: note = misc_feature - mFlt3l_MB
MTVLAPAWSPNSSLLLLLLLLSPCLRGTPDCYFSHSPISSNFKVKFRELTDHLLKDYPVTVAVNLQDEKHCKALWSLFLAQRWIEQLKTVAGSKMQTLLEDVNTEIHFVTSCTFQPLPECLRFVQTNISHLLKDTCTQLLALKPCIGKACQNFSRCLEVQCQPDSSTLLPPRSPIALEATELPEPRPRQLLLLLLLLLPLTLVLLAAAWGLRWQRARRRGELHPGVPLPSHP

SEQ ID NO: 32 - protein, 235 aa, Homo sapiens
  REGION 1..235: note = misc_feature - hFLT3LG_MB
MTVLAPAWSPTTYLLLLLLLSSGLSGTQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPQPPLLLLLLLPVGLLLLAAAWCLHWQRTRRRTPRPGEQVPPVPSPQDLLLVEH